(19) United States
(12) Patent Application Publication
(10) Pub. No.: US 2025/0332260 A1
(43) Pub. Date: Oct. 30, 2025

(54) IMMUNE COMPATIBLE CELLS FOR ALLOGENEIC CELL THERAPIES TO COVER GLOBAL, ETHNIC, OR DISEASE-SPECIFIC POPULATIONS

(71) Applicant: Garuda Therapeutics, Inc., Cambridge, MA (US)

(72) Inventors: Dhvanit Shah, Chestnut Hill, MA (US); Gurinder Singh, Cambridge, MA (US)

(21) Appl. No.: 19/176,071

(22) Filed: Apr. 10, 2025

Related U.S. Application Data
(60) Provisional application No. 63/632,155, filed on Apr. 10, 2024.

(51) Int. Cl. A61K 40/50; A61K 40/11; A61K 40/22; C12N 9/22; C12N 15/11; C12N 15/90

(52) U.S. Cl. CPC A61K 40/50; A61K 40/11; A61K 40/22; C12N 9/226; C12N 15/11; C12N 15/907; C12N 2310/20

(57) ABSTRACT

In the various aspects and embodiments, the present disclosure provides cell populations or cell “banks” thereof to provide immune compatible, allogeneic cell therapies. In the various aspects and embodiments, the cell populations and progeny thereof maintain sufficient HLA Class I and HLA Class II functionalities, while facilitating patient matching to prevent or reduce graft versus host disease (GVHD) or graft rejection. The disclosure further provides methods for creating the populations by gene editing, and methods for cell therapy involving cells or tissues derived from the cell populations (including but not limited to hematopoietic stem cells, or “HSCs”, progenitors, or progenies thereof).

### PRIORITY

This application claims priority to, and the benefit of, U.S. provisional application No. 63/632,155 filed Apr. 10, 2024, which is hereby incorporated by reference in its entirety.

### SEQUENCE LISTING

The instant application contains a Sequence Listing which has been submitted in XML format via Patent Center and is hereby incorporated by reference in its entirety. Said XML copy, created on Jul. 18, 2025, is named 065724-520001US.xml and is 30,168 bytes in size.

### BACKGROUND

Cellular therapies, based on cells derived from allogeneic (derived from healthy donor), autologous (derived from patients), and/or induced pluripotent stem cells (iPSCs), have enormous potential for medical applications to regenerate cells and tissues. However, the use of allogeneic or autologous cells (generated from cells of an intended recipient) will not be practical in most instances. Meanwhile, transplant of cells or tissues produced from allogeneic cells face issues of immune rejection and/or Graft Versus Host Disease (GVHD), for example, caused by significant HLA mismatching. Likewise, transplant of autologous or allogeneic cells faces concerns about consistency, scalability, durability and affordability. Further, it is not feasible to prepare iPSC stocks representing enough HLA haplotypes to cover a significant portion of the population, based on current HLA matching standards. Cell banks that are HLA modified to provide “off-the-shelf” cell therapies, and which provide an ease of matching with a substantial portion of the population, and which are consistent, scalable, and of lower cost are of great need. In various aspects and embodiments, the present disclosure provides HLA-modified cells and collections thereof to meet these and other objectives.

### SUMMARY OF THE DISCLOSURE

In the various aspects and embodiments, the present disclosure provides cell populations or cell “banks” thereof to provide immune compatible, allogeneic cell therapies covering global, ethnic, and disease-specific populations. In the various aspects and embodiments, the cell banks and progeny thereof maintain sufficient HLA Class I and HLA Class II functionalities, while facilitating patient matching to prevent or reduce graft versus host disease (GVHD) or graft rejection. The disclosure further provides methods for creating the cell banks by gene editing, and methods for cell therapy involving cells or tissues derived from the cell banks (including but not limited to hematopoietic stem cells, or “HSCs”, progenitors, or progenies thereof).

In the various aspects and embodiments, the present disclosure provides an HLA-modified cell population that is HLA-A^{neg}, HLA-DPB1^{neg}, and HLA-DQA1^{neg}, wherein the cell is homozygous for, or comprises a single copy of, HLA-C*07:01, HLA-BRB1*03:01, and HLA-B*08:01. In some embodiments, the cell population is heterozygous for HLA-DPA1 and homozygous for, or comprise a single copy of, HLA-DRB1 and HLA-DRB3. In various embodiments, the cell population has a haplotype described herein. In variations, the modified cell population that is HLA-A^{neg}, HLA-DPB1^{neg}, and HLA-DQA1^{neg}, with alleles of HLA-C, HLA-BRB1, and/or HLA-B other than HLA-C*07:01, HLA-BRB1*03:01, or HLA-B*08:01, respectively. The modified cell population may retain HLA-B, HLA-C, and/or HLA-DRB1.

In the various aspects and embodiments, the cell population is a human stem cell or human progenitor cell population. In some embodiments, the stem cell is a pluripotent stem cell, which may be a human induced pluripotent stem cell (hiPSC). In various embodiments the iPSCs are derived from peripheral blood CD34+ cells. In some embodiments, the stem cell population is a hematopoietic stem cell (HSC) population (e.g., differentiated from the iPSCs), or a cell population derived therefrom. The HSC population may be differentiated from iPSCs by contacting cells with a Piezo1 agonist, such as Yoda1. In some embodiments, the cell population comprises cells that are a hematopoietic cell lineage, such as a hematopoietic lineage selected from common lymphoid precursor (CLP) cells, granulocyte-monocyte progenitor (GMP) cells, progenitor-T cells, T lymphocytes, B lymphocytes, Natural Killer cells, neutrophils, monocyte, macrophages, red cells, megakaryocytes, and platelets. In still other embodiments, the cell population is a non-hematopoietic cell population, e.g., differentiated from the iPSCs ex vivo. Exemplary cells include, but are not limited to, mesenchymal stem cell, neural stem cell, or epithelial stem cell. In some embodiments, the non-hematopoietic cell is selected from neurons, astrocytes, oligodendrocytes, cardiomyocytes, skeletal muscle cells, hepatocytes, pancreatic β cells, and lung epithelial cells, or progenitors thereof.

In other aspects, the present disclosure provides a method for cell therapy, comprising, administering to a recipient in need thereof a cell population or tissue derived from the cell population disclosed herein. In the various embodiments, the cell population is matched for the retained classical HLA. For example, in embodiments where the cell population retains HLA-B, HLA-C, and HLA-DRB1, the administered cell population or tissue is matched with the recipient at one or more (or all) of HLA-B, HLA-C and HLA-DRB1.

In other aspects, the invention provides a method for cell therapy (or uses of the cell compositions for cell therapy), comprising administering a cell population described herein, or pharmaceutically acceptable composition thereof, to a human subject in need thereof. In various embodiments, the methods described herein are used to treat blood (malignant and non-malignant), bone marrow, and immune diseases. In various embodiments, the human subject has a condition comprising one or more of lymphopenia, a cancer, infectious disease (e.g., viral disease such as HPV or HIV) an immune deficiency, an autoimmune disease, a skeletal dysplasia, hemoglobinopathies, an anemia, a bone marrow failure syndrome, and a genetic disorder (e.g., a genetic disorder impacting the immune system).

In various embodiments an HSC population is administered to the recipient, or in other embodiments, the cell population is a hematopoietic cell lineage differentiated (e.g., ex vivo) from the HSC population. In other embodiments, the cell population is a non-hematopoietic lineage differentiated from the iPSCs described herein. In the various embodiments, the subject has a condition selected from a hematological malignancy, aplastic anemia, hemoglobinopathy, inborn error of metabolism, and severe immunodeficiency. Other conditions and disorders to be treated are disclosed herein and include lymphopenia, cancer, immune deficiency, autoimmune disease, skeletal dysplasia, a bone marrow failure syndrome, and genetic disorder impacting the immune system.

In some embodiments, the subject is a tissue or organ transplant recipient. In some embodiments, the subject is experiencing or is at risk for GVHD. Organs that can be transplanted, for example, include the heart, kidneys, liver, lungs, pancreas, intestine, and thymus, among others. Tissues for transplant can include, for example, bones, tendons (both referred to as musculoskeletal grafts), bone marrow or HSCs, cornea, skin, heart valves, nerves and/or veins.

In one aspect, the present disclosure provides a method for making a cell population of the present disclosure, where the method comprises providing an iPSC population and modifying the iPSC population to prepare an HLA-modified iPSC population that is HLA-A^{neg}, HLA-DPB1^{neg}, and HLA-DQB1^{neg}. In various embodiments, the iPSC population is homozygous for or comprises a single gene for one or more (or all) of HLA-B, HLA-C, HLA-DRB1, HLA-DQA1, and HLA-DRB3, and/or heterozygous for HLA-DPA1. The method further comprises preparing embryoid bodies (EBs) from the iPSC population; dissociating the EBs and enriching for CD34+ cells to prepare a CD34+-enriched cell population; and inducing endothelial-to-hematopoietic transition (EHT) of the CD34+-enriched cell population to prepare a population comprising hematopoietic stem cells (HSCs) and/or hematopoietic stem progenitor cells (HSPCs). In some embodiments, the method may further comprise harvesting CD34+ cells from the population comprising HSCs and/or HSPCs to enrich for a population undergoing EHT. In some embodiments, the method further comprises differentiating the cell population undergoing EHT to a hematopoietic lineage.

In the various embodiments, the iPSC is HLA-modified using CRISPR-Cas9, CRISPR-Cas12, STAR-CRISPR, CRISPR-CasX, CRISPR-associated transposase, zinc-finger nuclease, RNA editor, insulated genomic domain-platform editing, or combinations thereof. In the various embodiments, the iPSC is HLA-modified using a CRISPR-Cas9 endonuclease and one or more guide RNAs (gRNAs) as ribonucleoprotein.

In various aspects, the present disclosure provides a method for making an HLA-modified cell of the present disclosure, where the method comprises contacting a cell with a Cas endonuclease and one or more guide RNAs (gRNAs) targeting the Cas endonuclease to one or more HLA-specific or HLA allele-specific regions. In embodiments, HLA-modification includes the use of any method for introducing nucleic acid into cells, including for example electroporation, lipid reagent, or sonoporation (sonication). Exemplary gRNA to target certain HLA haplotypes are described herein.

In various embodiments, the CD34+ enrichment and endothelial-to-hematopoietic transition is induced at Day 7 to Day 15 of iPSC differentiation. In embodiments the induction of endothelial-to-hematopoietic transition comprises increasing the expression or activity of dnmt3b, such as, but not limited to, by Piezo1 activation. Other methods for inducing EHT are described herein. In the various embodiments, the CD34+-enriched cells undergoing EHT are differentiated to one or more of common lymphoid precursor (CLP) cells, granulocyte-monocyte progenitor (GMP) cells, progenitor-T cells, T lymphocytes, B lymphocytes, Natural Killer cells, neutrophils, monocyte, macrophages, red cells, megakaryocytes, and platelets. In the various embodiments the CD34+-enriched cells undergoing EHT are differentiated ex vivo to progenitor T cells, T cells, or NK cells.

Other aspects and embodiments of this disclosure will be apparent from the following detailed disclosure and working examples.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows the coverage for most frequent haplotypes (based on HLA-C, HLA-B, and DRB1) in the U.S. Two haplotypes provide cumulative coverage of about 22%, while about 50 haplotypes provide cumulative coverage of about 70% of the U.S. population.

FIG. 2 illustrates an HSC cell bank differentiated from iPSCs that are gene edited to knockout HLA genes.

FIG. 3A and FIG. 3B show that iPSC-derived HSCs that are derived with Piezo1 activation undergo pro-T cell differentiation similar to bone marrow (BM)-HSCs. FIG. 3A is a FACS plot of differentiation efficiency to CD34+CD7+ pro T cells of Bone Marrow (BM) HSCs and iPSC-HSCs derived with Piezo1 activation. FIG. 3B is a quantification of CD34+CD7+ cells (%) derived with (1) BM-HSCs and (2) iPSC-HSCs (Piezo1 Activation). FIG. 3B shows the average of three experiments.

FIG. 4A and FIG. 4B show that iPSC-derived HSCs generated with Piezo1 activation undergo T cell differentiation and such T cells can be activated with CD3/CD28 beads similar to T cells derived from BM-HSCs. FIG. 4A is a FACS plot of activation efficiency (CD3+CD69+ expression) of T cells differentiated from BM-HSCs and iPSC-derived HSCs generated with Piezo1 activation. FIG. 4B is a quantification of CD3+CD69+ cells (%) derived with (1) BM-HSCs and (2) iPSC-HSCs (Piezo1 Activation). FIG. 4B shows the average of three experiments.

FIG. 5 shows that iPSC-derived HSCs can differentiate to functional T cells. IFNγ expression is a consequence of T cell activation after T cell receptor (TCR) stimulation via CD3/CD28 beads. IFNγ expression in T cells differentiated from iPSC-derived HSCs, generated upon Piezo1 activation, enhances HSC ability to further differentiate to functional cells (e.g., cells). FIG. 5 shows the average of three experiments.

FIGS. 6A-6C show: generation of three CCR5-knockout (KO) iPSC clones (FIG. 6A), that CCR5-KO does not affect the iPSC pluripotency (FIG. 6B), and that CCR5-KO does not affect the ability of cells to undergo the endothelial-to hematopoietic transition (FIG. 6C).

FIGS. 7A-7C show: generation of three CD33-KO iPSC clones (FIG. 7A), that CD33-KO does not affect the ability of cells to undergo the endothelial-to hematopoietic transition (FIG. 7B), and that CD33-KO does not affect the ability of cells to generate self-renewing HSCs (FIG. 7C).

FIG. 8A shows the presence of HLA genes located on the short arm of chromosome 6. FIG. 8B shows a schematic representation of targeting the HLA-A, HLA-DQB1, and HLA-DPB1 genes using gRNAs. Exons are illustrated by horizontal arrows; vertical arrows denote locations of gRNA targeting. Genomic coordinates are shown in parentheses.

FIGS. 9A and 9B show the phenotype analysis of triple knockout (HLA edited) cells performed by FACS and immunofluorescence. FIG. 9A shows the overall expression of HLA class-I molecules (HLA-A, HLA-B, and HLA-C) on the cell surface, where the HLA edited cells are positive for overall HLA class-I expression to a similar degree as wild-type cells. FIG. 9B shows cell expression of HLA-A via immunofluorescence, where HLA-A is not expressed in the HLA edited clone.

FIG. 10 shows that the HLA edited clones preserve their pluripotency (maintain trilineage differentiation), as illustrated by immunofluorescence, with ectoderm differentiation indicated by NESTIN-488 and PAX6-594 staining, mesoderm differentiation indicated by GATA-488 staining, and endoderm differentiation indicated by CXCR4-488 and FOX2A-594 staining.

FIG. 11 shows the immune compatibility of the HLA edited HSCs. HLA edited HSCs and control HSCs (WT, B2M KO, and HLA Class II null) were co-cultured with peripheral blood mononuclear cells (PBMCs) matching HLA-B and HLA-C, but with mismatched HLA-A. The PBMC-CD8+, and NK cell-mediated cytotoxicity was measured by an annexin V staining assay.

FIG. 12 shows in vivo engrafting potential of HLA edited HSCs. Equal proportions of mCherry HLA edited HSCs and wild-type HSCs were mixed for a competitive transplant into mice, where bone marrow (BM) and peripheral blood samples were evaluated by FACS to compare the relative amounts of each cell type present in the samples.

FIGS. 13A and 13B show that WT and HLA-edited HSCs can differentiate to Pro-T Cells (FIG. 13A), as identified by a combination of CD34-CD7+ and CD34+CD7+ markers. FIG. 13B graphically represents the results shown in FIG. 13A.

FIG. 14 shows that WT and HLA-edited HSCs can differentiate to the NK cell lineage, as identified by CD3-CD56+ markers.

FIGS. 15A and 15B show that WT and HLA-edited HSCs can differentiate to the monocyte/macrophage lineage, which also preserves the overall expression of both class I and class II molecules as identified by CD11b+CD14+ markers (FIG. 15A). FIG. 15B shows analysis of HLA-I and HLA-II on cells gated on CD11b+CD14+.

FIGS. 16A to 16C show that HLA-DQB1 and HLA-DPB1 deletion does not affect the expression of other HLA Class II molecules. FIG. 16A is a schematic showing differentiation of HLA-edited iPSCs to macrophages. FIG. 16B is an immunofluorescence experiment confirming the specific deletion of the DPB1 and DQB1 molecules. FIG. 16C shows that the same cells preserve the class II DRB1 expression.

FIGS. 17A and 17B show that deletion of HLA-A does not impact Class I peptide presentation. FIG. 17A shows a schematic representation of immunopeptidome analysis. FIG. 17B shows results of the immunopeptidome analysis, which reveals that little difference exists in the numbers of peptides and representative proteins presented by class I molecules of WT and HLA-edited cells.

FIGS. 18A and 18B show that deletion of HLA-DP and DQ does not impact Class II peptide presentation. FIG. 18A shows immunopeptidome analysis scheme. FIG. 18B shows that despite the deletion of HLA-DP and DQ, the cells preserve their ability to present a broad spectrum of peptide through HLA Class II.

FIG. 19 is a schematic representation of in vivo testing of antigen-mediated immune response: Delayed Type Hypersensitivity Assay (DTH), sensitizing stage and elimination stage respectively.

FIGS. 20A and 20B show that HLA-edited HSCs reconstitute a functional immune system as demonstrated by DTH reaction in immune deficient mice. FIG. 20A shows a delayed-type hypersensitivity assay on transplanted mice were performed, which is an assay that involves the cross-talk of different types of immune cells. Specifically, mice were sensitized by subcutaneous injection of sheep Red blood cells (antigen). A functional immune system results in the swelling of the left paw that was measured with a micro caliper. As can be seen in FIG. 20A, the non-transplant mice did not show any left paw swelling as they are immunodeficient. Conversely, the mice transplanted with Cord Blood CD34+ cells show tissue swelling and doubled the diameter of their left paw. FIG. 20B is a graphical evaluation of the results shown in FIG. 20A.

FIG. 21 shows HSC-derived T cells can be activated in vitro. Top panel shows FACS analysis of activated T cells from different sources, including from HSCs prepared according to the present disclosure. T cells of the present disclosure demonstrate comparable or superior activation as measured by increased CD107 expression. The lower panel shows Dynabeads activation, where activated T cells express inflammatory cytokines. HSC-derived T cells express higher levels of inflammatory cytokines as exemplified by TNF-alpha and interferon gamma expression levels.

FIG. 22 shows that CCR5-knocked out HSCs can comparably differentiate into pro-T cells, compared to their wild type (gHSC) counterpart HSC (CCR5 retained).

FIG. 23 shows CCR5-knocked out HSCs can comparably differentiate into double positive (CD4+CD8+) T cells when compared to their wild type (gHSC) counterpart HSCs (CCR5 retained).

FIG. 24 shows that HSCs generated according to this disclosure (D8+7 iPSC-CD34+ cells, with and without Yoda 1, “Y”) successfully differentiate into CD4+CD8+ (“double positive”) T cells as well as TCR α/β T cells. The methods of the present disclosure substantially outperform T cell maturation from bone marrow CD34+ cells.

FIG. 25 shows that HSCs generated according to this disclosure (D8+7 iPSC-CD34+ cells, with or without Y) successfully rearrange TCR, and outperform bone marrow CD34+ cells.

FIG. 26 shows the HSC differentiation potential into T cell subtypes. After a 35-day differentiation period pro-T cells were evaluated by cell sorting for the presence of CD4+, CD8+, and AB+ T cell populations. FIG. 26 (right) compares the differentiation potential of bone marrow-derived CD34+ cells, embryoid body CD34+ cells, and HSCs prepared according to the present disclosure (e.g., using Piezo1 activation).

FIG. 27 shows the degree of T-cell mediated cytotoxicity measured from a co-culture of HSC-derived T cells with CD19+ lymphoma cells in the presence of an anti-CD3/CD-19 bispecific antibody. T cells prepared from HSCs according to the present disclosure demonstrate a high level of cytotoxicity against the target cells.

FIG. 28 shows that HSC-derived T cells (pro-T cells) can be transduced with high efficiency. Pro-T cells underwent lentiviral (LV) transduction with an anti-CD-19 chimeric antigen receptor (CAR) transgene (left), where the efficiency of LV transduction was measured by cell sorting based on anti-CD19 scFv staining (right). Results indicate that HSC-derived T cells achieved approx. 85% transduction efficiency.

FIG. 29 shows that LV-transduced HSC-derived T cells (pro-T cells) can effectively mature into CD4+/CD8+ T cells via CAR transduction.

FIG. 30 shows the ability of anti-CD19 CAR-transduced HSC-derived T cells (CAR pro-T cells) to function via receptor-mediated cytotoxicity. Luc+ NALM6 leukemia cells were co-cultured with CAR pro-T cells and cell-mediated cytotoxicity was measured by luciferase assay.

FIG. 31 shows the ability of the HSCs to develop into pro-T cells as measured by their CD34-CD7+ markers.

FIGS. 32A and 32B demonstrates increased expression of T cell-specific transcription factors and Thymus engrafting molecules with the pro-T cells derived from HSCs according to the instant disclosure. FIG. 32A shows TCF7 mRNA expression and FIG. 32B shows CCR7 mRNA expression.

FIGS. 33A and 33B shows that HSC-derived Pro-T Cells engraft and differentiate in thymus. FIG. 33A illustrates the engraftment and analysis procedure. FIG. 33B shows FACS analysis of CD3 cell population of cells gated on CD45+ cell population, which shows the superior engraftment and differentiation potential of the HSC-derived Pro-T Cells in the thymus.

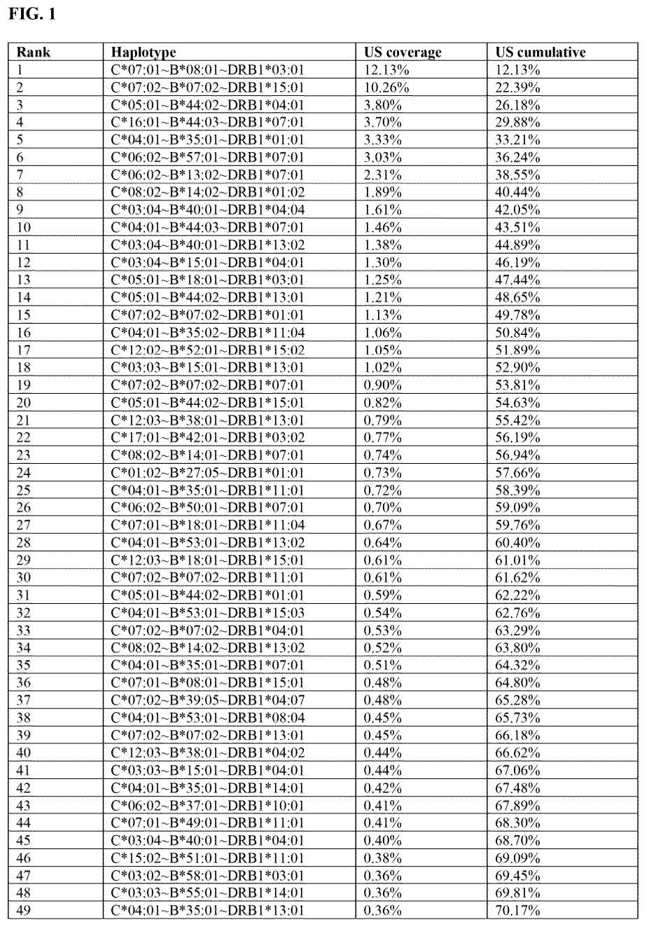

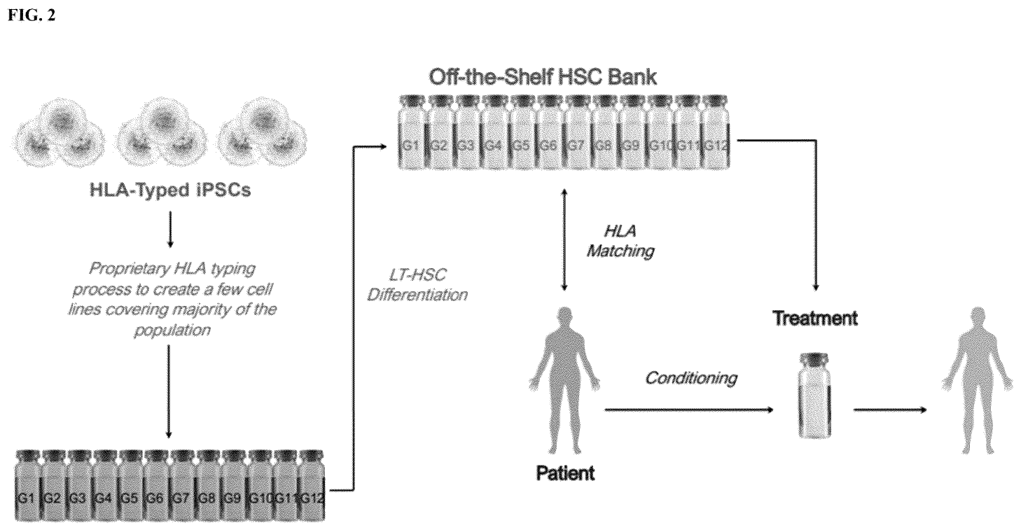

The term “gHSC” is used herein to refer to the iPSC-derived hematopoietic stem cells of the present disclosure.

The terms “wild type” (WT), “unedited”, “non-HLA-edited” are used interchangeability herein to refer to the non-gene edited cells of the present disclosure.

EB34+ cells refer to Embryonic body derived CD34+ cells. These comprise hemogenic endothelial cells.

### DETAILED DESCRIPTION

In the various aspects and embodiments, the present disclosure provides cell populations or cell “banks” and collections thereof to provide immune compatible, allogeneic cell therapies covering global, ethnic, and disease-specific populations. In the various aspects and embodiments, the cell banks and progeny thereof maintain sufficient HLA Class I and HLA Class II functionalities, while facilitating patient matching to prevent or reduce graft versus host disease (GVHD) or graft rejection. The disclosure further provides methods for creating the cell banks by gene editing, and methods for cell therapy involving cells or tissues derived from the cell banks (including but not limited to hematopoietic stem cells, or “HSCs”, as well as progenitors and progenies thereof).

In an aspect, the disclosure provides an HLA-modified cell population that is HLA-A^{neg}, HLA-DPB1^{neg}, and HLA-DQB1^{neg} and/or is homozygous for, or retains a single gene for, HLA-B. HLA-C, and HLA-DRB1. In embodiments, the cell population is homozygous or heterozygous for HLA-DQA1 and HLA-DPA1.

In various embodiments, the HLA-modified cell is HLA-A^{neg} where the cell (e.g., iPSC) is homozygous for HLA-A*01:01 and the cell is then HLA-modified by engineering a disruption in each HLA-A gene by a Cas-targeted gRNA. In some embodiments, the HLA-A gene is disrupted by targeting a sequence within exon 2. In various embodiments, the targeted disruption is performed using a gRNA comprising a nucleotide sequence selected from Table 2. In some embodiments, the gRNA to disrupt HLA-A comprises a nucleic acid sequence of GAGGGTTCGGGGCGCCATGA (SEQ ID NO: 6). In some embodiments, the disruption to the HLA-A gene comprises a deletion, insertion, or indel, which results in a cell phenotype of ablated expression of HLA-A in comparison to a cell which has not undergone the genetic modification. In embodiments, the deletion, insertion, or indel creates a frameshift and/or a stop codon.

In various embodiments, the HLA-modified cell is HLA-DPB1^{neg} where the cell (e.g., iPSC) is homozygous or heterozygous for HLA-DPB1*01:01 or HLA-DPB1*04:01 and the cell is then HLA-modified by engineering a disruption in each HLA-DPB1 gene by a Cas-targeted gRNA. In some embodiments, the HLA-A gene is disrupted by targeting a sequence within exon 2. In various embodiments, the targeted disruption is performed using a gRNA comprising a nucleotide sequence selected from Table 2. In some embodiments, the gRNA to disrupt HLA-DPB1 comprises a nucleic acid sequence of GGAGAGATACATCTACAACC (SEQ ID NO: 21). In some embodiments, the disruption to the HLA-DPB1 gene comprises a deletion, insertion, or indel, which results in a cell phenotype of ablated expression of HLA-DPB1 in comparison to a cell which has not undergone the genetic modification. In embodiments, the deletion, insertion, or indel creates a frameshift and/or a stop codon.

In various embodiments, the HLA-modified cell is HLA-DQB1^{neg} where the cell (e.g., iPSC) is homozygous for HLA-DQB1*02:01 and the cell is then HLA-modified by engineering a disruption in each HLA-DQB1 gene using a Cas-targeted gRNA. In some embodiments, the HLA-DQB1 gene is disrupted by targeting a sequence within exon 2. In various embodiments, the targeted disruption is performed using a gRNA comprising a nucleotide sequence selected from Table 2. In some embodiments, the gRNA to disrupt HLA-DQB1 comprises a nucleic acid sequence of GTGCTACTTCACCAACGGGA (SEQ ID NO: 26) or AGGTCGTGCGGAGCTCCAAC (SEQ ID NO: 27). In some embodiments, the disruption to the HLA-DQB1 gene comprises a deletion, insertion, or indel, which results in a cell phenotype of ablated expression of HLA-DQB1 in comparison to a cell which has not undergone the genetic modification. In embodiments, the deletion, insertion, or indel creates a frameshift and/or a stop codon.

In some embodiments, the HLA-modified cell comprises a deletion of one or both genes for HLA-DQB2 and/or HLA-DQB3. In some embodiments, the HLA-modified cell is homozygous for, or comprises a single copy of, HLA-DQB2 and/or HLA-DQB3.

In various embodiments, the HLA-modified cell is homozygous for, or comprises a single copy of, HLA-B. In embodiments, the HLA-B allele is HLA-B*08:01. In various embodiments, the cell is homozygous for, or comprises a single copy of, HLA-C. In embodiments, the HLA-C allele is HLA-C*07:01. In embodiments where the cell contains a single copy of an HLA gene, the cell may be heterozygous for the HLA gene, where one copy of the gene is disrupted by gene editing. See PCT/US2023/076083, which is hereby incorporated by reference in its entirety.

In various embodiments, one or both DRB1 alleles are maintained. In various embodiments, the cell is homozygous for, or comprises a single copy of, HLA-DRB1. In embodiments, the DRB1 allele is HLA-DRB1*03:01.

In various embodiments, one or both DPA1 alleles are maintained. In some embodiments, the HLA-modified cell is heterozygous for DPA1, or unchanged at the DPA1 loci. In embodiments, the cell comprises one or more of the DPA1 alleles HLA-DPA1*01:03 and HLA-DPA1*02:01.

In various embodiments, one or both DQA1 alleles are maintained. In some embodiments, the HLA-modified cell is unchanged at the DQA1 loci. In some embodiments, the HLA-modified cell is homozygous for DQA1, or comprises a single copy of DQA1. In embodiments, the cell comprises the DQA1 allele HLA-DQA1*05:01.

In various embodiments, one or both DRB3 alleles are maintained. In some embodiments, the HLA-modified cell is unchanged at the DRB3 loci. In some embodiments, the HLA-modified cell is homozygous for DRB3, or comprises a single copy of DRB3. In embodiments, the cell comprises the DRB3 allele HLA-DRB3*01:01.

In embodiments, the HLA-modified cell is HLA-A^{neg}, HLA-DPB1^{neg}, and HLA-DQA1^{neg}; homozygous for HLA-B*08:01, HLA-C*07:01, and HLA-DRB1*03:01. In embodiments, the cell is unmodified at other HLA loci, and may comprise for example one or more of the following alleles: HLA-DQA1*05:01, HLA-DRB3*01:01, HLA-DPA1*01:03, and HLA-DPA1*02:01.

The cell lines are either homozygous for the DRB1 gene or are edited to have only a single DRB1 gene. In various embodiments, the cell is also homozygous for one or more isoforms of the DR Gene, such as but not limited to, DRB2, DRB4, and DRB5 genes, or are edited to have only a single copy of one or more of DRB2, DRB4, and DRB5 genes. In still other embodiments, DRB2, DRB4, and DRB5 are retained and unmodified (and may be homozygous or heterozygous in some embodiments). Alternatively, in embodiments, the HLA-modified cell comprises both copies of one or more of DRB2, DRB4, and DRB5 deleted or inactivated.

In some embodiments, the cell population is homozygous at HLA-E or one HLA-E gene is deleted or inactivated. In some embodiments, HLA-E is unmodified, and may be homozygous or heterozygous.

In some embodiments, the cell population is homozygous at HLA-F or one HLA-F gene is deleted or inactivated. In some embodiments, HLA-F is unmodified, and may be homozygous or heterozygous.

In some embodiments, the cell population is homozygous at HLA-G or one HLA-G gene is deleted or inactivated. In some embodiments, HLA-G is unmodified, and may be homozygous or heterozygous.

In various embodiments, the cell population is a stem cell population, such as a pluripotent stem cell. In some embodiments, the cell population is a human induced pluripotent stem cell (hiPSC). As described in further detail herein, iPSCs may be derived from cord blood, bone marrow biopsy, mobilized peripheral blood derived hCD34+ cells, human CD34+ cells, immune cells, immune progenitor cells, hematopoietic cells, non-hematopoietic cells (e.g., cells that can differentiate into cells such as fibroblasts, osteoblasts, chondrocytes, myocytes, endothelial cells, and neurons), and banked organ derived cells. In embodiments, iPSCs are created from CD34+ cells isolated from peripheral blood. In various embodiments, as described further below, primary cells are reprogrammed to generate human iPSC cell bank(s), which can be HLA-modified to generate off-the-shelf therapeutics containing immune compatible, allogeneic human cells.

In some embodiments, the stem cell population is a hematopoietic stem cell (HSC) population or a hematopoietic stem progenitor cell (HSPC) population, or a cell population derived therefrom. As described in further detail herein, the cell population may be, or may be used to derive, a hematopoietic cell lineage. For example, the hematopoietic lineage may be selected from common lymphoid precursor (CLP) cells, granulocyte-monocyte progenitor (GMP) cells, progenitor-T cells, T lymphocytes, B lymphocytes, Natural Killer cells, neutrophils, monocyte, macrophages, dendritic cells, red cells, megakaryocytes, and platelets.

In still other embodiments, the cell population is a non-hematopoietic stem cell population. The population can be derived from iPSCs, or may be donor or patient derived. Exemplary non-hematopoietic stem cells include mesenchymal stem cell, neural stem cell, or epithelial stem cell. In still other embodiments, the cell population is, or is used to derive, a non-hematopoietic cell, such as a cell selected from fibroblasts, osteoclasts, chondrocytes, myocytes, cardiomyocytes, endothelial cells, neurons, astrocytes, oligodendrocytes, hepatocytes, pancreatic β cells, and lung epithelial cells, or progenitors thereof.

In some embodiments, the cell population has a DRB1 haplotype of DRB1*03:01. In some embodiments, the cell population has an HLA-C haplotype of C*07:01. In some embodiments, the cell population has an HLA-B haplotype of B*08:01. In some embodiments, the cell population comprises an HLA-C˜HLA-B˜DRB1 haplotype of C*07:01˜B*08:01˜DRB1*03:01.

In accordance with the various embodiments, the cell line is immune compatible at two, four, six, eight, ten, or twelve HLA loci by either matching at certain HLA haplotypes or not mismatching at certain HLA haplotypes.

For example, the cell line is immune compatible at HLA-C by virtue that the cell line is homozygous at HLA-C (and HLA-C is matched), or one copy of HLA-C is matched and another copy of HLA-C is deleted or inactivated. The cell line is immune compatible at HLA-A by virtue that both HLA-A genes are deleted or inactivated (i.e., the cell line is HLA-A^{neg}). The cell line is also immune compatible at HLA-DRB1 by virtue that the cell line is homozygous at HLA-DRB1 (and thus HLA-DRB1 is matched), or one copy of HLA-DRB1 is matched and another copy of HLA-DRB1 is deleted or inactivated.

In various embodiments, the cell lines is immune compatible at HLA-B by virtue that the cell line is homozygous at HLA-B, or one copy of HLA-B is matched and another copy of HLA-B is deleted or inactivated.

In some embodiments the cell lines is immune compatible at HLA-DPB1, because both copies of DPB1 are deleted or inactivated (HLA-DPB1^{neg}).

In some embodiments the cell lines is immune compatible at HLA-DQB1, because both copies of DQB1 are deleted or inactivated (HLA-DQB1^{neg}).

The cell line can be immune compatible at HLA-E by virtue that the cell line is homozygous at HLA-E, or one copy of HLA-E is matched and another copy of HLA-E is deleted or inactivated. However, in some embodiments HLA-E is retained as unmodified, and is either matched or not matched.

In some embodiments, the cell line is developed by deleting or inactivating specific HLA haplotypes using gene editing techniques, including but not limited to CRISPR-Cas9, while preserving other HLA haplotypes. For example, cell lines can be derived from human primary cells from a homozygous donor (at one or more loci), and/or by deleting one copy of mismatched haplotype. Non limiting examples of sgRNA for use with CRISPR-Cas9 gene editing systems are described herein. The sgRNAs can be used singly, or in combinations to induce gene edits, such as double strand breaks, in exon 1 and/or exon 2 of the target HLA, leading to inactivation, mutagenesis, or deletions of one base or more, such as 5 bases or more, or 10 bases or more, or 50 bases or more, or 100 bases or more, or 500 bases or more, sufficient to functionally inactivate the target gene or eliminate its functional expression. In some embodiments, the gRNA targeting domains are 16, 17, 18, 19, 20, 21, 22, 23, 24, 25, 26 or more nucleotides in length. In some embodiments, the gRNAs comprise a modification at or near the 5′ end (e.g., within 1-10, 1-5, or 1-2 nucleotides of the 5′ end) and/or a modification at or near the 3′ end (e.g., within 1-10, 1-5, or 1-2 nucleotides of the 3′ end). In some embodiments, the modified gRNAs exhibit increased resistance to nucleases. In some embodiments, a gRNA comprises two separate RNA molecules (i.e., a “dual gRNA”). A dual gRNA comprises two separate RNA molecules: a “crispr RNA” (or “crRNA”) and a “tracr RNA” and is well known to one of skill in the art.

The cell lines comprise one or more HLA modifications (e.g., one or more HLA gene deletions) to facilitate HLA matching with a recipient, to make cell therapies available to a diverse population with a universal collection of HLA matching cell lines (i.e., as compared to a non-HLA-modified collection encumbered by enormous diversity of HLA haplotypes in a population). In an aspect, the disclosure provides a collection of cell lines (or “cell populations”) comprising at least two cell lines, where the cell lines in the collection represent at least two different HLA haplotypes. For example, each cell line comprises a deletion or inactivation of HLA-A genes (HLA-A^{neg}), in addition to being HLA-DPB1^{neg} and HLA-DQB1^{neg}, while being homozygous for, or comprising a single copy of, HLA-B, HLA-C, and HLA-DRB1. Other HLA modifications to Class I and/or Class II genes are made according to the present disclosure to facilitate immune-compatibility matching with a recipient without compromising the safety or efficacy of the cell therapy.

The Major Histocompatibility complex (MHC) system, also referred to herein as human leukocyte antigen (HLA), is comprised of a polymorphic gene cluster located on the short arm of chromosome 6 (6p21.3). HLA includes regions designated as class I and class II. The main function of HLA class I gene products is to present endogenous (i.e., intracellular) peptides to cognate CD8+ (cytotoxic) T Cells. The main function of HLA class II molecules is to present peptide antigens from exogenous proteins to CD4+ helper T Cells. HLA class I gene products are critical for detecting and targeting cells that develop deleterious mutations and/or cancers, as well as for detecting and targeting cells harboring intracellular pathogens including viruses. HLA class II gene products are critical for detecting the presence of pathogens in a tissue environment and coordinating an immune response against the pathogen. While HLA class I gene products are expressed on most cells, HLA class II genes are largely expressed by professional antigen presenting cells such as dendritic cells, macrophages, and B cells. HLA class II molecules are also known to be expressed by some T cells as well as subsets of epithelial and endothelial cells, for example. Kambayashi and Laufer, Atypical MHC class II-expressing antigen-presenting cells: can anything replace a dendritic cell? Nature Reviews Immunology vol. 14:719-730 (2014).

HLA class I molecules comprise HLA-A, HLA-B, HLA-C, HLA-E, HLA-F, and HLA-G, which differ substantially in their level of polymorphism. HLA class I molecules are comprised of a single polypeptide complexed with β2-microglobulin (B2M). Indeed, knock out of B2M can abolish functional expression of HLA-class I gene products. There are about 7,453 identified HLA-A alleles, about 8,849 identified HLA-B alleles, about 7,393 identified HLA-C alleles, about 310 identified HLA-E alleles, about 50 identified HLA-F alleles, and about 102 identified HLA-G alleles. See hla.alleles.org. Natural killer (NK) cells recognize cells lacking HLA class I expression, a phenomenon often observed in a wide spectrum of tumor types. Malmberg K., Immune selection during tumor checkpoint inhibition therapy paves way for NK-cell “missing self” recognition, Immunogenetics vol. 69, pages 547-556 (2017). Generally, HLA-A and HLA-B exhibit the highest expression among class I molecules.

HLA class II molecules comprise two transmembrane polypeptide chains (a and B) forming the antigen binding cleft. HLA molecules corresponding to class II include HLA-DP, HLA-DM, HLA-DOA, HLA-DOB, HLA-DQ, and HLA-DR, and which have highly varying levels of polymorphism (see hla.alleles.org). HLA class II genes include those with “classical” class II alpha and beta chain genes of HLA-DP, -DQ and -DR, and “non-classical” loci such as HLA-DM and -DO. DRB1 shows the highest diversity among class II genes and is highly expressed.

TABLE 1
below summarizes HLA class I and class II genes.

| HGNC ID | Symbol | Name | Chromosome |
|---|---|---|---|
| HGNC: 4931 | HLA-A | major histocompatibility complex, class I, A | 6p22.1 |
| HGNC: 4932 | HLA-B | major histocompatibility complex, class I, B | 6p21.33 |
| HGNC: 4933 | HLA-C | major histocompatibility complex, class I, C | 6p21.33 |
| HGNC: 4934 | HLA-DMA | major histocompatibility complex, class II, DM alpha | 6p21.32 |
| HGNC: 4935 | HLA-DMB | major histocompatibility complex, class II, DM beta | 6p21.32 |
| HGNC: 4936 | HLA-DOA | major histocompatibility complex, class II, DO alpha | 6p21.32 |
| HGNC: 4937 | HLA-DOB | major histocompatibility complex, class II, DO beta | 6p21.32 |
| HGNC: 4938 | HLA-DPA1 | major histocompatibility complex, class II, DP alpha 1 | 6p21.32 |
| HGNC: 4939 | HLA-DPA2 | major histocompatibility complex, class II, DP alpha 2 | 6p21.32 |
| | | (pseudogene) | |
| HGNC: 19393 | HLA-DPA3 | major histocompatibility complex, class II, DP alpha 3 | 6p21.32 |
| | | (pseudogene) | |
| HGNC: 4940 | HLA-DPB1 | major histocompatibility complex, class II, DP beta 1 | 6p21.32 |
| HGNC: 4941 | HLA-DPB2 | major histocompatibility complex, class II, DP beta 2 | 6p21.32 |
| | | (pseudogene) | |
| HGNC: 4942 | HLA- | major histocompatibility complex, class II, DQ alpha 1 | 6p21.32 |
| | DQA1 | | |
| HGNC: 4943 | HLA- | major histocompatibility complex, class II, DQ alpha 2 | 6p21.32 |
| | DQA2 | | |
| HGNC: 4944 | HLA- | major histocompatibility complex, class II, DQ beta 1 | 6p21.32 |
| | DQB1 | | |
| HGNC: 4945 | HLA- | major histocompatibility complex, class II, DQ beta 2 | 6p21.32 |
| | DQB2 | | |
| HGNC: 4946 | HLA- | major histocompatibility complex, class II, DQ beta 3 | 6p21.3 |
| | DQB3 | | |
| HGNC: 4947 | HLA-DRA | major histocompatibility complex, class II, DR alpha | 6p21.32 |
| HGNC: 4948 | HLA- | major histocompatibility complex, class II, DR beta 1 | 6p21.32 |
| | DRB1 | | |
| HGNC: 4950 | HLA- | major histocompatibility complex, class II, DR beta 2 | 6p21.3 alternate |
| | DRB2 | (pseudogene) | reference locus |
| HGNC: 4951 | HLA- | major histocompatibility complex, class II, DR beta 3 | 6p21.3 alternate |
| | DRB3 | | reference locus |
| HGNC: 4952 | HLA- | major histocompatibility complex, class II, DR beta 4 | 6p21.3 alternate |
| | DRB4 | | reference locus |
| HGNC: 4953 | HLA- | major histocompatibility complex, class II, DR beta 5 | 6p21.32 |
| | DRB5 | | |
| HGNC: 4954 | HLA- | major histocompatibility complex, class II, DR beta 6 | 6p21.32 |
| | DRB6 | (pseudogene) | |
| HGNC: 4955 | HLA- | major histocompatibility complex, class II, DR beta 7 | 6p21.3 alternate |
| | DRB7 | (pseudogene) | reference locus |
| HGNC: 4956 | HLA- | major histocompatibility complex, class II, DR beta 8 | 6p21.3 alternate |
| | DRB8 | (pseudogene) | reference locus |
| HGNC: 4957 | HLA- | major histocompatibility complex, class II, DR beta 9 | 6p21.32 |
| | DRB9 | (pseudogene) | |
| HGNC: 4962 | HLA-E | major histocompatibility complex, class I, E | 6p22.1 |
| HGNC: 4963 | HLA-F | major histocompatibility complex, class I, F | 6p22.1 |
| HGNC: 4964 | HLA-G | major histocompatibility complex, class I, G | 6p22.1 |
| HGNC: 4965 | HLA-H | major histocompatibility complex, class I, H | 6p22.1 |
| | | (pseudogene) | |
| HGNC: 4967 | HLA-J | major histocompatibility complex, class I, J | 6p22.1 |
| | | (pseudogene) | |
| HGNC: 4969 | HLA-K | major histocompatibility complex, class I, K | 6p22.1 |
| | | (pseudogene) | |
| HGNC: 4970 | HLA-L | major histocompatibility complex, class I, L | 6p22.1 |
| | | (pseudogene) | |
| HGNC: 19406 | HLA-N | major histocompatibility complex, class I, N | 6p22.1 |
| | | (pseudogene) | |
| HGNC: 21196 | HLA-P | major histocompatibility complex, class I, P | 6p22.1 |
| | | (pseudogene) | |
| HGNC: 19395 | HLA-S | major histocompatibility complex, class I, S | 6p21.33 |
| | | (pseudogene) | |
| HGNC: 23478 | HLA-T | major histocompatibility complex, class I, T | 6p22.1 |
| | | (pseudogene) | |
| HGNC: 23477 | HLA-U | major histocompatibility complex, class I, U | 6p22.1 |
| | | (pseudogene) | |
| HGNC: 23482 | HLA-V | major histocompatibility complex, class I, V | 6p22.1 |
| | | (pseudogene) | |
| HGNC: 23425 | HLA-W | major histocompatibility complex, class I, W | 6p22.1 |
| | | (pseudogene) | |
| HGNC: 19385 | HLA-X | major histocompatibility complex, class I, X | 6p21.3 |
| | | (pseudogene) | |
| HGNC: 33913 | HLA-Y | major histocompatibility complex, class I, Y | 6p21.33 |
| | | (pseudogene) | |
| HGNC: 19394 | HLA-Z | major histocompatibility complex, class I, Z | 6p21.32 |
| | | (pseudogene) | |

For transplantation of organs and tissues (including hematopoietic stem cells) from allogeneic donors, the main criterion for donor selection is HLA compatibility. Particularly for HSC transplantation, a new lympho-hematopoietic system must develop in the recipient to replace the recipient's diseased lympho-hematopoietic system. Immunological reactions are substantially driven by T cells and include host-versus-graft (HVG) responses, which refers to patient cell reactivity against donor cells, and graft-versus-host (GVH) responses, which refers to donor lymphocyte reactivity against host tissues. The immunotherapeutic effect on neoplastic cells is often referred to as graft-versus-leukemia (GVL). GVH (or GVHD) can be associated with severe side effects in transplant recipients (e.g., HSCT) and is largely responsible for HSC transplant-related morbidity and mortality.

Molecular HLA typing conventionally involves typing the α1 and α2 domains for class I and the α1 domain for class II. Donors are generally selected based on typing of the classical HLA genes HLA-A, HLA-B, HLA-C, HLA-DRB1, HLA-DQB1, and HLA-DPB1. For example, US standards conventionally attempt to match 8 loci (both alleles for HLA-A, HLA-B, HLA-C, and HLA-DRB1), while European standards involve matching 10 loci (both alleles for HLA-A, HLA-B, HLA-C, HLA-DRB1, and HLA-DQB1).

In an aspect, the present disclosure provides a method of generating off-the-shelf cell populations or “banks” of cells. The method comprises: (i) providing donor or patient-derived cells and/or pluripotent stem cells (e.g., iPSCs); and (ii) modifying in vitro one or more endogenous coding sequences in the cell (e.g., iPSCs) genome, thereby knocking out one or more genes or mutating one or more genes to encode a nonfunctional protein in the cell population (e.g., iPSCs). The modified cells in various embodiments are identified as: HLA-A⁻B⁺C⁺DP⁻DR⁺DQ⁻. For retained HLA (for example, HLA-B, HLA-C, and HLA-DR), cells can be homozygous, or retain only a single copy of the gene. In embodiments, the modified cells are identified at least as (a) HLA-B+, HLA-C+, and HLA-DR+.

As used herein, the term “neg” or (−) with respect to a particular HLA Class I or Class II gene indicates that both copies of the gene have been disrupted in the cell line or population, and thus the cell line or population does not display significant functional expression of the gene. Such cells can be generated by full or partial gene deletions.

In accordance with aspects and embodiments of this disclosure, cell populations or cell banks are provided to allow for harvest from allogeneic donor or patient or for the generation (e.g., by ex vivo expansion or differentiation) of hematopoietic stem cells (HSCs) and progenitors or progenies thereof for off-the shelf cell and tissue therapies. The cells are gene edited to delete particular HLA genes (as described), to thereby facilitate immune compatible matching for an intended recipient. As used herein, the term “delete” in this context refers to a genetic modification of the target gene (i.e., gene edit) that abrogates functional expression of the corresponding gene product (i.e., the corresponding polypeptide). Such gene edits include full or partial gene deletions, or deletions of critical cis-acting expression control sequences. In accordance with embodiments of this disclosure, expression of B2M is not altered, and expression of class II major histocompatibility complex transactivator (CIITA) is not altered, because these modifications would abolish HLA expression. B2M expression is critical for functional expression of HLA class I, and CIITA is critical for HLA class II expression. For example, alteration of B2M risks a response by natural killer (NK) cells and is potentially harmful for the proliferation of cells that are infected by pathogens or are oncogenic. Alteration of CIITA risks lack of antigen-presentation abilities though class II HLAs. For HSC transplantation (for example), some level of functional class I and class II expression is required to reconstitute immunological surveillance.

In various aspects and embodiments, the present disclosure provides HLA class I and/or HLA class II modified cells in which certain HLA gene(s) (as described) have been altered or deleted to make the cells immune compatible for the cell-based therapy, in a subject in need of such therapy, without being encumbered by concerns of the harmful effects of HLA incompatibility or donor matching. Advantageously, these alterations in the class I and class II molecules can enhance the biocompatibility of these cells in diverse populations, including those originating from Asia, Europe, Africa, South America, and North America as they manifest all of the characteristics of unmodified cells, except that they advantageously eliminate or ameliorate harmful or toxic functions in therapeutical applications of their HLA-unmodified counterpart cells.

In various aspects and embodiments, this disclosure minimizes the HLA loci required for haplotype matching, including for HSC transplantation. In various aspects and embodiments, the cells, or cells or tissues derived therefrom, exhibit functional class II antigen presentation (i.e., class II antigen presentation is not substantially impaired by lower class II expression and/or class II diversity in comparison to non-HLA modified cells or tissues). In various aspects and embodiments, the cells, or cells or tissues derived therefrom, do not show substantial susceptibility to oncogenesis or viral infection (i.e., due to loss of class I expression or class I diversity). In various aspects and embodiments, the cells, or cells or tissues derived therefrom, are not substantially targeted by the innate immune system of the recipient (e.g., NK cells) due to loss of HLA expression or diversity (i.e., as compared to non-HLA modified cells or tissues).

HLA haplotypes are indicated herein according to convention. HLA alleles can be named by indicating the locus, antigenic specificity, and molecularly typed allele group. The asterisk “*” sign indicates that typing is performed by a molecular method and the colon “:” is a field separator. For example, where A*03:01 is an allele of interest, the first field (A*03) refers to a group of alleles that encode for the A3 antigen, and the second field (:01) refers to a particular allele that encodes the unique HLA protein A*03:01. Homozygous alleles can include one or more polymorphisms in one or both copies in some embodiments (that is, need not be identical).

The cell populations or banks can be modified for one or more additional functionalities (as described in more detail herein), including deletion or insertion of additional genes. For example, the cell lines can be modified to express or overexpress certain cytokines, suicide genes, T-cell receptor, one or more chimeric antigen receptors (CARs), and/or combinations thereof. In some embodiments, cell populations or banks, or progeny thereof, are modified such that certain endogenously expressed genes are deleted, inactivated, or reduced in expression, such as but not limited to genes encoding CCR5 or miR-155, or genes encoding cell surface markers including but not limited to CD33, CLL, CD19, CD7, and/or CD38.

In various embodiments, cells are inserted with a nucleic acid encoding a CAR specific to myeloma, leukemia or lymphoma targets, including but not limited to CD19, CD33, and BCMA. In various embodiments, the cells are introduced with nucleic acid encoding tandem CARs, including but not limited CD38/IL3 and CD20/CD19. In various embodiments, the cells are introduced with nucleic acid encoding disease specific dual CAR, Quad CAR, or tandem-CARs,

Thus, non-limiting examples include but are not limited to: (i) cells deleted for CCR5 to generate CCR5-deleted cellular therapies of HIV-AIDS patients; (ii) cells deleted for CD33 to generate CD33-deleted cellular therapies for treating leukemia and/or lymphoma patients; (iii) cells deleted for CD33 to generate CD33-deleted cellular therapies for use in connection with CAR-T, CAR-NK, CAR-T progenitor cells, or CAR-macrophage cells for treating leukemia and/or lymphoma patients.

The cell populations, or cells derived therefrom (e.g., progeny) can be used with FDA approved CAR-T therapy, such as, Tisagenlecleucel, also known as tisa-cel (Kymriah), Axicabtagene ciloleucel, also known as axi-cel (Yescarta), Brexucabtagene autoleucel, also known as brexu-cel (Tecartus), Lisocabtagene maraleucel, also known as liso-cel (Breyanzi), Idecabtagene vicleucel, also known as ide-cel (Abecma), Ciltacabtegene autoleucel, also known as cilta-cel (Carvykti), or any other CAR-T therapy which damage the normal cells during their therapeutic applications.

In one aspect, the disclosure provides a collection of cell populations (i.e., cell banks comprising at least two populations of cells) of expanded primary cells, derivatives of iPSC cells, or stem cell lines, where the cell lines in the collection represent a single HLA-C haplotype. Each cell population comprises a deletion or inactivation of both HLA-A genes. In various embodiments, the cell populations are either homozygous for the HLA-C gene or are edited to have only a single HLA-C gene (e.g., by deletion of one HLA-C gene). In such embodiments, the collection comprises cell populations or banks having the HLA-C haplotype: C*07:01.

In some embodiments, the alleles are matched with DMA*01, DMB*01, DOA*01, DOB*01, HFE*001, MICA*002, MICA*007, MICA*008, MICA*009, MICA*010, MICA*012, MICA*018, MICA*019, MICB*002, MICB*004, MICB*005, TAP1*01, TAP1*02, TAP1*03, TAP1*04, TAP1*05, TAP1*06, TAP2*01, or TAP2*02.

In some embodiments, the cell population in the collection retain at least one HLA-B gene, and represent a single HLA-B haplotype. In some embodiments, the cell lines are either homozygous for an HLA-B gene or are edited to have only a single HLA-B gene. In such embodiments, the collection comprises cell populations or banks having the HLA-B haplotype: B*08:01.

In some embodiments, the collection comprises cell populations or banks with the following haplotypes: C*07:01˜B*08:01

In various embodiments, the collection comprises cell populations or banks with at least the following DRB1 haplotypes: DRB1*03:01. The cell lines are either homozygous for the DRB1 gene or are edited to have only a single DRB1 gene. In various embodiments, the cell populations are also homozygous for one or more isoforms of the DR Gene, such as but not limited to, DRB2, DRB4, and DRB5 genes, or are edited to have only a single copy of one or more of DRB2, DRB4, and DRB5 genes. In still other embodiments, DRB2, DRB4, and DRB5 are retained and unmodified (and may be homozygous or heterozygous across the cell lines in some embodiments).

In various embodiments, the collection comprises cell populations or banks with the following haplotype: C*07:01˜B*08:01˜DRB1*03:01.

In various embodiments, the cell populations or banks retain HLA-E, HLA-F, and HLA-G genes, which can be homozygous or heterozygous across the collection (e.g., are unmodified).

In some embodiments, the cell populations or banks are HLA-DPB1^{neg}, while retaining DP genes selected from DPA1, DPA2, DPA3, and DPB2. In some embodiments, the cell populations or banks have a deletion or inactivation of both HLA-DPB1 genes and/or one or both DPA2, DPA3, and DPB2 genes. In such embodiments, the cell populations or banks are heterozygous for HLA-DPA1, having the alleles HLA-DPA1*01:03 and HLA-DPA1*02:01. In some embodiments, the cell lines or banks retain DPA2, DPA3, and DPB2 (and which are unmodified, and may be homozygous or heterozygous across the cell populations or banks).

In some embodiments, the cell populations or banks are HLA-DQB1^{neg}, while retaining DQ genes selected from DQA1, DQA2, DQB2, and DQB3. In some embodiments, the cell populations or banks have a deletion or inactivation of both HLA-DQB1 genes and/or one or both DQA1, DQA2, DQB2, and DQB3 genes. In embodiments, the populations or banks are homozygous for, or retain a single copy of, DQA1, having the allele DQA1*05:01. In some embodiments, the cell lines or banks retain DQA2, DQB2, and DQB3 (and which are unmodified, and may be homozygous or heterozygous across the cell populations or banks).

In exemplary embodiments, the cell populations or banks have both HLA-A genes deleted, both DPB1 genes, and both DQB1 genes disrupted or deleted. In such embodiments, the cell lines are homozygous for, or retain only single copies of, HLA-B, HLA-C, HLA-DRB1, and optionally HLA-DQA1, and HLA-DRB3. In such embodiments, the cell lines are further homozygous or heterozygous for HLA-DPA1. In embodiments, both copies of all other HLA genes (particularly those not annotated as pseudogenes) are retained, and these genes may be homozygous or heterozygous.

In various embodiments, the cell populations are induced pluripotent stem cell (iPSC) lines. In some embodiments, the cell populations are hematopoietic stem cell (HSC) lines. For example, HSC populations may be prepared from iPSCs (having the desired gene deletions or inactivations) by a method described herein. In various embodiments, the iPSC population is a human iPSC population derived from lymphocytes, cord blood cells, peripheral blood mononuclear cells, CD34+ cells (e.g., as isolated from peripheral blood), or human primary tissues, as described herein.

In some embodiments, cells are derived from the HSCs for administration to a recipient, and the cells may be any of the hematopoietic lineages. For example, the hematopoietic lineage may be selected from common lymphoid precursor (CLP) cells, granulocyte-monocyte progenitor (GMP) cells, progenitor-T cells, T lymphocytes, B lymphocytes, Natural Killer cells, neutrophils, monocyte, macrophages, dendritic cells, red cells, megakaryocytes, and platelets. T cells may be CD4+ helper T cells, CD8+ cytotoxic T cells, or regulatory T cells (Tregs).

In some embodiments, the cells are non-hematopoietic stem cells or precursor cells, or cells differentiated therefrom. Exemplary stem cells include mesenchymal stem cells, neural stem cells, and epithelial stem cells. In various embodiments, iPSC lines are used to produce various non-hematopoietic cells and tissues, including those selected from neurons (including cortical, dopaminergic, and motor neurons), astrocytes, oligodendrocytes, cardiomyocytes, cornea, chondrons, skeletal muscle cells, hepatocytes, pancreatic β cells, and lung epithelial cells. Protocols for deriving such cells and tissues are known in the art.

In various embodiments, the cell populations in the bank are each contained within separate containers suitable for maintaining viability of the cell lines or cell compositions for expansion, differentiation, or administrations. The cell composition of this disclosure may further comprise a pharmaceutically acceptable carrier or vehicle suitable for intravenous infusion or other administration route (including cell or tissue engraftment), and the composition may include a suitable cryoprotectant. An exemplary, on-limiting, carrier is DMSO (e.g., about 10% DMSO).

In other aspects, this disclosure provides a method for cell therapy. The method comprises administering to a recipient in need thereof a cell population derived from a cell population of this disclosure. In various embodiments, the administered cell population or tissue is matched with the recipient for HLA-C. In embodiments, the cell population or tissue is further matched with the recipient for HLA-B. In some embodiments, the cell population or tissue is further matched with the recipient for DRB1. The cell population need not be matched to the recipient for HLA-A, DPB1, or DQB1. In various embodiments, all other loci are unmatched.

In some embodiments, a method for treating a subject according to the present disclosure comprises: (a) expanding a population of pluripotent stem cells (e.g., iPSCs) according to the methods described herein; (b) preparing HSCs or progenies thereof (as described herein); and (c) introducing the population of hematopoietic stem cells or progenies thereof into the subject. Optionally, the hematopoietic stem cells may be differentiated into common megakaryocyte-erythroid progenitor cells, lymphoid progenitor cells, progenitor T and/or B cells, common myeloid progenitor cells, granulocytes, granulocyte-megakaryocyte progenitor cells, promyelocytes, basophils, eosinophils, neutrophils, erythrocytes, reticulocytes, thrombocytes, megakaryoblasts, platelet-producing megakaryocytes, platelets, monocytes, macrophages, dendritic cells, microglia, osteoclasts, lymphocytes, NK cells, B-cells and/or T-cells prior to their administration.

In some embodiments, the cell populations are used to generate cell therapies to treat human diseases including but not limited to, a hematological malignancy, aplastic anemia, hemoglobinopathy, inborn error of metabolism, and severe immunodeficiency. For example, the subject may have a condition selected from acute myeloid leukemia; acute lymphoblastic leukemia; chronic myeloid leukemia; chronic lymphocytic leukemia; acute lymphatic leukemia, aplastic anemia, Krabbe Disease, bone marrow failure syndromes, Hurler Syndrome, Leukodystrophies, Myelodysplastic syndromes, POEMS syndrome, Primary amyloidosis, myeloproliferative disorder; myelodysplastic syndrome; multiple myeloma; Non-Hodgkin lymphoma; Hodgkin disease; aplastic anemia; pure red-cell aplasia; paroxysmal nocturnal hemoglobinuria; Fanconi anemia; thalassemia major; sickle cell anemia; severe combined immunodeficiency (SCID); acquired immune deficiency syndrome (AIDS); Wiskott-Aldrich syndrome; hemophagocytic lymphohistiocytosis; inborn errors of metabolism; epidermolysis bullosa; severe congenital neutropenia; Shwachman-Diamond syndrome; Diamond-Blackfan anemia; eukocyte adhesion deficiency; X-linked forms of SCID, Sickle cell anemia, Alpha thalassemia, Beta thalassemia, Delta thalassemia, Hemoglobin E/thalassemia, Hemoglobin S/thalassemia, Hemoglobin C/thalassemia, Hemoglobin D/thalassemia, Chronic granulomatous disease, X-linked Chronic granulomatous disease, autosomal recessive (AR) chronic granulomatous disease, chronic granulomatous disease AR I NCF1, Chronic granulomatous disease AR CYBA, Chronic granulomatous disease AR II NCF2, Chronic granulomatous disease AR III NCF4, X-linked Severe Combined Immune Deficiency (SCID), IL7-RA SCID, CD3 SCID, Rag1/Rag2 SCID, ADA SCID, Artemis SCID, CD45 SCID, Jak3 SCID, Congenital agranulocytosis, Congenital agranulocytosis-congenital neutropenia-SCN1, Congenital agranulocytosis-congenital neutropenia-SCN2, Familial hemophagocytic lymphohistiocystosis (FHL), Familial hemophagocytic lymphohistiocytosis type 2 (FHL2, perforin mutation), Agammaglobulinemia (X-linked Agammaglobulinemia), Wiskott-Aldrich syndrome, Chediak-Higashi syndrome, Hemolytic anemia due to red cell pyruvate kinase deficiency, Paroxysmal nocturnal hemoglobinuria, X-linked Adrenoleukodystrophy (X-ALD), X-linked lymphoproliferative disease, Acquired idiopathic sideroblastic anemia, Systemic mastocytosis, Von willebrand disease (VWD), Congenital dyserythropoietic anemia type 2, Cartilage-hair hypoplasia syndrome, Unicentric Castleman's Disease, Multicentric Castleman's Disease, Congenital amegakaryocytic thrombocytopenia (CAMT) type I, Reticular dysgenesis, Hereditary spherocytosis, Blackfan-Diamond syndrome, Shwachman-Diamond syndrome, Mucopolysaccharidoses, Lesch-Nyhan syndrome, Glycogen storage disease, Congenital mastocytosis, Omenn syndrome, X-linked Immunodysregulation, polyendocrinopathy, Thrombocytopenia-absent radius syndrome, Osteopetrosis, Infantile osteopetrosis, and enteropathy (IPEX), IPEX characterized by mutations in FOXP3, X-linked syndrome of polyendocrinopathy, immune dysfunction, and diarrhea (XPID), X-Linked Autoimmunity-Allergic Dysregulation Syndrome (XLAAD), IPEX-like syndrome, Hyper IgM type 1, Hyper IgM type 2, Hyper IgM type 3, Hyper IgM type 4, Hyper IgM type 5, X linked hyperimmunoglobulin M, Bare lymphocyte Syndrome type I, or Bare lymphocyte Syndrome type II, myasthenia gravis, rheumatoid arthritis, multiple sclerosis, type I diabetes mellitus, idiopathic inflammatory myopathy, systemic lupus erythematosus (SLE), myasthenia gravis, Grave's disease, dermatomyositis, polymyositis, Crohn's disease, ulcerative colitis, gastritis, Hashimoto's thyroiditis, asthma, psoriasis, psoriatic arthritis, dermatitis, systemic scleroderma and sclerosis, inflammatory bowel disease (IBD), respiratory distress syndrome, meningitis, encephalitis, uveitis, glomerulonephritis, eczema, atherosclerosis, leukocyte adhesion deficiency, Raynaud's syndrome, Sjorgen's syndrome, Reiter's disease, Beheet's disease, immune complex nephritis, IgA nephropathy, IgM polyneuropathies, immune-mediated thrombocytopenias e.g., ITP), acute idiopathic thrombocytopenia purpura, chronic idiopathic thrombocytopeni purpura, hemolytic anemia, lupus nephritis, atopic dermatitis, pemphigus vulgaris, opsoclonus-myoclonus syndrome, pure red cell aplasia, mixed cryoglobulinemia, ankylosing spondylitis, hepatitis C-associated cryoglobulinemic vasculitis, chronic focal encephalitis, bullous pemphigoid, hemophilia A, membranoproliferative glomerulonephritis, adult and juvenile dermatomyositis, adult polymyositis, chronic urticaria, primary biliary cirrhosis, neuromyelitis optica, Graves' dysthyroid disease, bullous pemphigoid, membranoproliferative glomerulonephritis, Churg-Strauss syndrome, juvenile onset diabetes, hemolytic anemia, atopic dermatitis, systemic sclerosis, Sjorgen's syndrome and glomerulonephritis, dermatomyositis, ANCA, aplastic anemia, autoimmune hemolytic anemia (AIHA), factor VIII deficiency, hemophilia A, autoimmune neutropenia, Castleman's syndrome, Goodpasture's syndrome, solid organ transplant rejection, graft versus host disease (GVHD), autoimmune hepatitis, lymphoid interstitial pneumonitis, HIV, bronchiolitis obliterans (non-transplant), Guillain-Barre Syndrome, large vessel vasculitis, giant cell (Takayasu's) arteritis, medium vessel vasculitis, Kawasaki's Disease, polyarteritis nodosa, Wegener's granulomatosis, microscopic polyangiitis (MPA), Omenn's syndrome, Alzheimer, chronic renal failure, acute infectious mononucleosis, or HIV and herpes virus associated diseases.

In some embodiments, the cell populations are used to generate cell therapies to treat cancer, including but not limited to, solid and hematological cancers, acquired diseases, congenital diseases, and non-hematopoietic diseases, including but not limited to diseases affecting neurons, astrocytes, oligodendrocytes, cardiomyocytes, skeletal muscle cells, hepatocytes, pancreatic β cells, lung epithelial cells, etc.

In some embodiments, an immune cell lineage (derived from cell populations or banks described herein) is administered to a patient in need thereof. For example, the immune cell lineage may be a T cell, NK cell, B-cell, or macrophage. In some embodiments, the T cell lineage is a T-regulatory cell or cytotoxic T cell. In some embodiments, the T cell expresses a heterologous TCR or a chimeric antigen receptor (CAR). In various embodiments, the recipient (patient) has a condition selected from one or more of lymphopenia, cancer, immune deficiency, autoimmune disease, skeletal dysplasia, a bone marrow failure syndrome, genetic disorder impacting the immune system, cardiac failure, neural disorders, immunodeficiency, blood disorders (e.g., Thalassemia, Anemias, sickle cell disease), heart disease, liver disease, multiple sclerosis, muscular dystrophy, skin and tissue regeneration, spinal cord degeneration, trauma, stroke, neurodegenerative diseases (e.g., Alzheimer, dementia, down's syndrome or Parkinson), metabolic disorder, hematopoietic stem cell transplant (HPSCT), i.e., administration of healthy hematopoietic stem cells to patients with dysfunctional or depleted bone marrow, thrombocytopenia, or cancer.

In various embodiments, the recipient has undergone lympho-deleting therapy, cyto-reductive therapy, or immunomodulatory therapy prior to administration of the cell therapy. In some embodiments, derivatives of the cell line(s) or banks of expanded primary cells (e.g., HSCs and/or progenies derived therefrom) disclosed herein are administered to reconstitute the recipient's hematopoietic system. Cell lineages generated using the methods described herein are administered to the subject e.g., by intravenous infusion. In some embodiments, the methods can be performed following myeloablative, non-myeloablative, or immunotoxin-based (e.g., anti-c-Kit, anti-CD45, etc.) conditioning regimes.

In still other embodiments, a cell or tissue derived from the cell population is administered to a recipient in need thereof. Exemplary cells include mesenchymal stem cells, neural stem cells, corneal epithelium/endothelium and RPE, epithelial stem cells, neuronal cells (or precursors thereof) (including cortical, dopaminergic, and motor neurons, or precursors thereof), astrocytes (or precursors thereof), oligodendrocytes (or precursors thereof), cardiomyocytes (or precursors thereof), skeletal muscle cells (or precursors thereof), hepatocytes (or precursors thereof), pancreatic β cells (or precursors thereof), and lung epithelial cells (or precursors thereof). Such cells can be administered to treat or ameliorate any disease or condition (including genetic or acquired condition) afflicting the relevant tissue or organ. Such tissues or organs include but are not limited to the central nervous system, skeletal muscle, heart, liver, pancreas, or lung.

In other aspects, this disclosure provides a method for making a cell population of the disclosure. The method can comprise providing an iPSC population that is HLA-modified according to this disclosure; enriching for CD34+ cells from a differentiated iPSC population (e.g., embryoid bodies, or EBs) to prepare a CD34+-enriched population; and inducing endothelial-to-hematopoietic transition of the CD34+-enriched population (e.g., for at least two days but no more than 12 days), to prepare a population comprising hematopoietic stem cells (HSCs) and/or hematopoietic stem progenitor cells (HSPCs). In some embodiments, the method further comprises harvesting a CD34+-enriched population that are undergoing endothelial-to-hematopoietic transition. For example, this can include harvesting of CD34+ floater and/or adherent cells, but generally will comprise at least non-adherent cells.

Conventionally, hematopoietic lineages are prepared by differentiation of iPSCs to embryoid bodies up to day 8 to harvest CD34+ cells. CD34 is commonly used as a marker of hemogenic endothelial cells, hematopoietic stem cells, and hematopoietic progenitor cells. In accordance with embodiments of this disclosure, endothelial-to-hematopoietic transition (EHT) is induced in a CD34+ cell population, and which can be derived from iPSC-embryoid bodies, and optionally used for the ex vivo generation of hematopoietic lineages.

In various embodiments, iPSCs are prepared by reprogramming somatic cells. The term “induced pluripotent stem cell” or “iPSC” refers to cells derived from somatic cells, such as skin, bone marrow, umbilical cord blood or peripheral blood cells that have been reprogrammed back into an embryonic-like pluripotent state. In some embodiments, iPSCs are generated from somatic cells such as (but not limited to) fibroblasts or PBMCs (or cells isolated therefrom). In some embodiments, the iPSCs are derived from lymphocytes (e.g., T-cells, B-cells, NK-cells, etc.), cord blood cells (including from CD3+ or CD8+ cells from cord blood), PBMCs, CD34+ cells, or other human primary tissues. In some embodiments, iPSCs are derived from CD34+ cells isolated from peripheral blood. iPSCs can be selected to have HLA alleles as described herein (e.g., see Table 4).

Somatic cells may be reprogrammed by expression of reprogramming factors selected from Sox2, Oct3/4, c-Myc, Nanog, Lin28, and klf4. In some embodiments, the reprogramming factors are Sox2, Oct3/4, c-Myc, Nanog, Lin28, and klf4. In some embodiments, the reprogramming factors are Sox2, Oct3/4, c-Myc, and klf4. Methods for preparing iPSCs are described, for example, in U.S. Pat. Nos. 10,676,165; 9,580,689; and 9,376,664, which are hereby incorporated by reference in their entireties. In various embodiments, reprogramming factors are expressed using well known viral vector systems, such as lentiviral, Sendai, or measles viral systems. Alternatively, reprogramming factors can be expressed by introducing mRNA(s) encoding the reprogramming factors into the somatic cells. Further still, iPSCs may be created by introducing a non-integrating episomal plasmid expressing the reprogramming factors, i.e., for the creation of transgene-free and virus-free iPSCs. Known episomal plasmids can be employed with limited replication capabilities and which are therefore lost over several cell generations. Alternative methods include minicircle vectors, PiggyBac transposons, and exosome incorporation.

In some embodiments, the iPSC population is gene edited to delete or inactivate one or more HLA genes. The selection of HLA genes for deletion or inactivation is as already described. The deletion or inactivation refers to a genetic modification of the target gene (i.e., gene edit) that abrogates functional expression of the corresponding gene product (i.e., the corresponding polypeptide). Such gene edits include full or partial gene deletions, as well as deletions of critical cis-acting expression control sequences. For example, with respect to HLA class I genes, deletions can include deletions of one or more extracellular domains such as α1, α2, and α3 domains. In some embodiments, HLA class I deletions include deletions of the transmembrane domain. With respect to HLA class II genes, deletions can include deletions of one or more extracellular domains such as α1 and/or α2, or β1 and/or β2. In some embodiments, HLA class II deletions include deletions of the transmembrane domain. In some embodiments, the HLA deletions comprise deletions of the entire coding sequence or substantially the entire coding sequence. In some embodiments, deletions are targeted toward exon 1 and/or exon 2 of HLA genes, and includes in various embodiments a deletion of at least 50 base pairs, at least 100 base pairs, at least 250 base pairs, or at least 500 base pairs. In some embodiments, HLA disruptions are targeted toward exon 2 of HLA genes (e.g., HLA-A, DPB1, and DQB1), which result in deletions, insertions, or indels (deletions and insertions). In still other embodiments, gene deletions or inactivations alter critical expression control sequences such as promoters, cis-acting sequences bound by transcriptional activators, or ribosomal binding sequences, to thereby substantially reduce or eliminate expression. In embodiments, the gene editing event results in a frameshift and/or premature stop codon, thereby abrogating expression of functional protein.

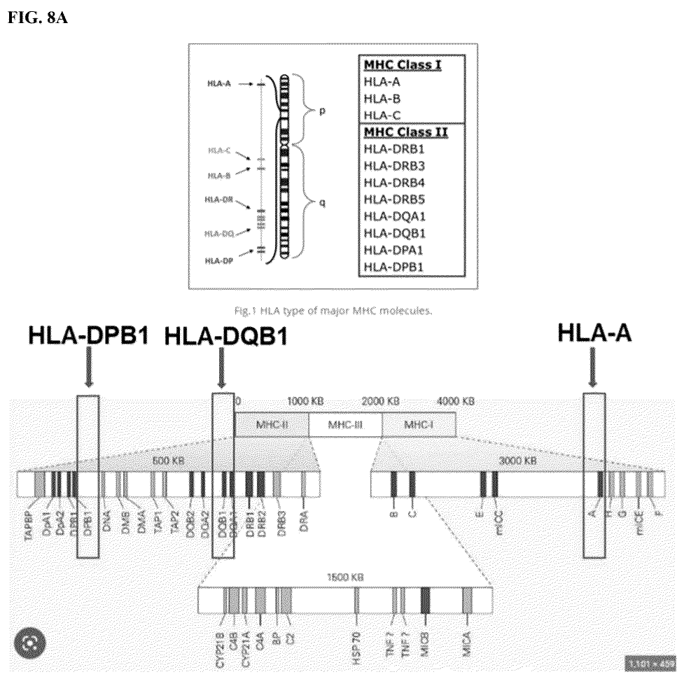

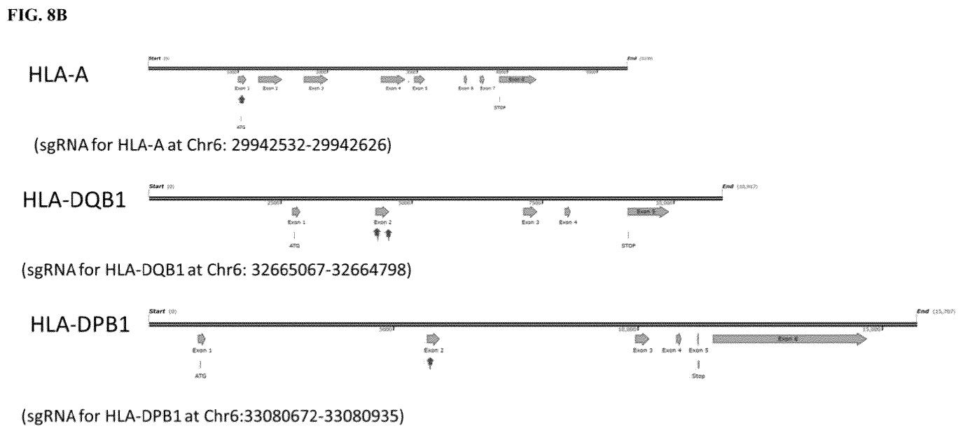

In various embodiments, a sgRNA targeting HLA-A can target a region of chromosome 6 defined as 29942532-29942626. In various embodiments, a sgRNA targeting HLA-DQB1 can target a region of chromosome 6 defined as 32665067-32664798. In various embodiments, a sgRNA targeting HLA-DPB1 can target a region of chromosome 6 defined as 33080672-33080935. See FIG. 8B.

The target HLA loci is deleted or inactivated using one or more gene modifying tools such as CRISPR-Cas (e.g., CRISPR-Cas9, CRISPR-Cas12, STAR-CRISPR, CRISPR-CasX, CRISPR-associated transposases), RNA-editors, insulated genomic domain-platform editing, and combinations thereof. In some embodiments, the target HLA loci can also be deleted or inactivated using siRNAs, oligonucleotides, and/or zinc finger nucleases. In some embodiments, the HLA modifications are conducted by CRISPR-Cas9, and which may employ one or a combination of gRNAs (e.g., sgRNAs) comprising a spacer sequence listed in Table 2 for the particular HLA gene and haplotype.

Generally, various editing technologies are known, which can be applied according to various embodiments of this disclosure. Gene editing technologies include but are not limited to zinc fingers (ZFs), and transcription activator-like effectors (TALEs), etc. Fusion proteins containing one or more of these DNA-binding domains and the cleavage domain of Fokl endonuclease can be used to create a double-strand break in a desired region of DNA in a cell (See, e.g., US Patent Appl. Pub. No. US 2012/0064620, US Patent Appl. Pub. No. US 2011/0239315, U.S. Pat. No. 8,470,973, US Patent Appl. Pub. No. US 2013/0217119, U.S. Pat. No. 8,420,782, US Patent Appl. Pub. No. US 2011/0301073, US Patent Appl. Pub. No. US 2011/0145940, U.S. Pat. Nos. 8,450,471, 8,440,431, 8,440,432, and US Patent Appl. Pub. No. 2013/0122581, the contents of all of which are hereby incorporated by reference). In some embodiments, gene editing is conducting using CRISPR associated Cas system (e.g., CRISPR-Cas9), as known in the art. See, for example, U.S. Pat. Nos. 8,697,359, 8,906,616, and 8,999,641, each of which is hereby incorporated by reference in its entirety. In various embodiments, the gene editing employs a Type II Cas endonuclease (such as Cas9) or employs a Type V Cas endonuclease (such as Cas12a). Type II and Type V Cas endonucleases are guide RNA directed. Design of gRNAs to guide the desired gene edit (while limiting or avoiding off target edits) is known in the art. See, for example, Mohr S E, et al., CRISPR guide RNA design for research applications, FEBS J. 2016 September; 283(17): 3232-3238. In still other embodiments, non-canonical Type II or Type V Cas endonucleases having homology (albeit low primary sequence homology) to S. pyogenes Cas9 or Prevotella and Francisella1 (Cpf1 or Cas12a) can be employed. Numerous such non-canonical Cas endonucleases are known in the art. Nidhi S, et al. Novel CRISPR-Cas Systems: An Updated Review of the Current Achievements, Applications, and Future Research Perspectives, Int J Mol Sci. 2021 April; 22(7): 3327. In still other embodiments, the gene editing employs base editing or prime editing to incorporate mutations without instituting double strand breaks. See, for example, Antoniou P, et al., Base and Prime Editing Technologies for Blood Disorders, Front. Genome Ed., 28 Jan. 2021; Matsuokas I G, Prime Editing: Genome Editing for Rare Genetic Diseases Without Double-Strand Breaks or Donor DNA, Front. Genet., 9 Jun. 2020. Various other gene editing processes are known, including use of dead Cas (dCas) systems (e.g., Cas fusion proteins) to target DNA modifying enzymes to desired targets using the dCas as a guide RNA-directed system. Brezgin S, Dead Cas Systems: Types, Principles, and Applications, Int J Mol Sci. 2019 December; 20(23): 6041.

Base editors that can install precise genomic alterations without creating double-strand DNA breaks can also be used in gene editing (e.g., designing gene therapy vectors) in the cells (e.g., iPSCs). Base editors essentially comprise a catalytically disabled nuclease, such as Cas9 nickase (nCas9), which is incapable of making DSBs and is fused to a nucleobase deaminase enzyme and, in some cases, a DNA glycosylase inhibitor. Currently, there are 2 major categories of base editors, cytidine base editors (CBEs) and adenine base editors (ABEs), which catalyze C>T and A>G transitions. Base editors can be delivered, for example, via HDAd5/35++ vectors to efficiently edit promoters and enhancer to active or inactivate a gene. Exemplary methods are described in U.S. Pat. Nos. 9,840,699; 10,167,457; 10,113,163; 11,306,324; 11,268,082; 11,319,532; and 11,155,803. Also contemplated are prime editors that comprise a reverse transcriptase conjugated to (e.g., fused with) a Cas endonuclease and a polynucleotide useful as a DNA synthesis template conjugated to (e.g., fused with) a guide RNA, as described in WO2020191153A2.

Exemplary vectors that can be used for the genome editing applications include, but are not limited to, plasmids, retroviral vectors, lentiviral vectors, adenovirus vectors (e.g., Ad5/35, Ad5, Ad26, Ad34, Ad35, Ad48, parvovirus (e.g., adeno-associated virus (AAV) vectors, herpes simplex virus vectors, baculoviral vectors, coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including herpes virus (e.g., Herpes Simplex virus types 1 and 2, Epstein-Barr virus, cytomegalovirus), and poxvirus (e.g., canarypox, vaccinia or modified vaccinia virus). The vector comprising the nucleic acid molecule of interest may be delivered to the cell (e.g., iPS cells, endothelial cells, hemogenic endothelial cells, HSCs (ST-HSCs or LT-HSCs) via any method known in the art, including but not limited to transduction, transfection, infection, and electroporation. Any of these vectors may include transposable element (such as a piggyback transposon or sleeping beauty transposon). Transposons insert specific sequences of DNA into genomes of vertebrate animals. The gene of interest can be integrated into the genome of a mammalian cell by transposase-catalyzed cleavage of similar excision sites that exist within the nuclear genome of the cell.

For increased efficiency, in some embodiments, the Cas and the gRNA are combined before being delivered into cells. The Cas-gRNA complex is known as a ribonucleoprotein (RNP). A number of methods have been developed for direct delivery of RNPs to cells. For example, RNP can be delivered into cells in culture by electroporation, or lipofection using a lipid-based regent (e.g., LIPOFECTAMINE), sonoporation or sonication, and microinjection. Electroporation using a nucleofection protocol is often preferred, as this allows the RNP to enter the nucleus of cells quickly, where it can immediately start cutting the genome. See, for example, Zhang S, Shen J, Li D, Cheng Y. Strategies in the delivery of Cas9 ribonucleoprotein for CRISPR/Cas9 genome editing. Theranostics. 2021 Jan. 1; 11 (2): 614-648, hereby incorporated by reference in its entirety. In some embodiments, Cas9 and gRNA are electroporated as RNP into the donor PBMC-derived iPSCs and/or HSCs.

Generally, a protospacer adjacent motif (PAM) is required for a Cas nuclease to cut and is generally found 3-4 nucleotides downstream from the cut site. The PAM is a short DNA sequence (usually 2-6 base pairs in length) that follows the DNA region targeted for cleavage by the CRISPR system, such as CRISPR-Cas9.

In some embodiments, the PAM sequences, sgRNAs, or base editing tools targeting haplotypes or polymorphs of HLA loci does not include four Gs, four Cs, GC repeats, or combinations thereof.

In some embodiments, a CRISPR/Cas9 system specific to a donor's unique HLA haplotype can be developed by designing singular gRNAs targeting each of the donor-specific HLA-A, HLA-DPB1, and HLA-DQB1 genes (for example), using the gRNAs as described herein. To perform genetic knockout, the gRNA targets the Cas9 protein to the appropriate site to edit. Next, the Cas9 protein can perform a double strand break (DSB), where the DNA repairs through a non-homologous end joining (NHEJ) mechanism which generates deletions, insertions, or indels resulting in a frameshift mutation and terminates the resulting protein's function. However, off-target genetic modifications can occur and alter the function of otherwise intact genes. For example, the Cas9 endonuclease can create DSBs at undesired off-target locations, even in the presence of some degree of mismatch. This off-target activity can create genome instability events, such as point mutations and genomic structural variations.

gRNAs can be used to develop clonal iPSCs from donor PBMCs. Such iPSC lines can be evaluated for (i) ON-target edit, (ii) OFF-target edits, and (iii) Translocation edits, for example using sequencing as described herein. Specifically, such assays can be performed by multiplex PCR with primers designed to target and enrich regions of interest followed by next-generation sequencing (e.g., Amplicon sequencing, AMP-seq). The ON-target panel and the translocation panel can amplify the intended edited region, allowing for selection of iPSC clones with the expected edits which are free from chromosomal translocation arising from unintended DSB cut-site fusion. The OFF-target panel can enrich any potential off-target regions identified via sequencing and allows for selection of iPSC clones with negligible off-target mutations. Together, these assays enable a screen of the iPSC clones to select the clones with the desired edits, while excluding potential CRISPR/Cas9-related genome integrity issues.

Although the differentiation potential of iPSCs has been demonstrated, some tissue-specific epigenetic memory from the starting material can interfere with iPSC differentiation. Therefore, in some embodiments, CD34+ cells can be selected for iPSC reprogramming, for example as described in Tobin S C and Kim K, “Generating pluripotent stem cells: differential epigenetic changes during cellular reprogramming,” FEBS Lett. 2012 Aug. 31; 586 (18): 2874-81, hereby incorporated by reference in its entirety. In some embodiments, to create transgene and virus-free iPSCs, the CD34+ cells are electroporated with episomal vectors to reprogram them into iPSCs. For example, using oriP/EBNA1 vectors, the episomal vectors can contain 5 reprogramming factors (e.g., Oct4, Sox2, Lin28, Klf4, and L-Myc) and replicate extra-chromosomally only once per cell cycle and are completely cleared out once the iPSC reach approx. passage 5-10. These embodiments do not include transient p53 suppression to maintain important safeguard checkpoints and reduces the risk of selecting clones with genomic instability. To further ensure the genomic stability and integrity of reprogrammed iPSCs, genetic and genomic assays can be performed to select for clones which, for example, did not undergo translocation and mutation events, and that did not integrate the episomal vectors.

In some embodiments, whole-genome sequencing (WGS) is performed on CD34+ cells and on iPSC clones after reprogramming, where the genomes are compared for differences arising from editing. These analyses provide an assessment of which iPSC clone genomes differ from the CD34+ starting material, enabling informed selection iPSC clones which did not accrue mutations during the reprogramming.

In some embodiments, karyotyping analyses using systems such as KARYOSTAT assays is used to select iPSC clones which did not accrue indels and translocation during the reprogramming, for example as described in Ramme A P, et al, “Supporting dataset of two integration-free induced pluripotent stem cell lines from related human donors,” Data Brief. 2021 May 15; 37:107140, hereby incorporated by reference in its entirety. KARYOSTAT assays allow for visualization of chromosome aberrations with a resolution similar to G-banding karyotyping. The size of structural aberration that can be detected is >2 Mb for chromosomal gains and >1 Mb for chromosomal losses. The KARYOSTAT array is functionalized for balanced whole-genome coverage with a low-resolution DNA copy number analysis, where the assay covers all 36,000 RefSeq genes, including 14,000 OMIM targets. The assay enables the detection of aneuploidies, submicroscopic aberrations, and mosaic events.

In some embodiments, Array Comparative Genomic Hybridization (aCGH) analyses is used to select iPSC clones which did not accrue copy number aberrations (CNA) during reprogramming, for example as described in Wiesner et al. “Molecular Techniques,” Editor(s): Klaus J. Busam, Pedram Gerami, Richard A. Scolyer, “Pathology of Melanocytic Tumors,” Elsevier, 2019, pp. 364-373, ISBN 9780323374576; and Hussein S M, et al. “Copy number variation and selection during reprogramming to pluripotency,” Nature. 2011 Mar. 3; 471 (7336): 58-62, hereby incorporated by reference in its entirety. aCGH is a technique that analyzes the entire genome for CNA by comparing the sample DNA to reference DNA.

In some embodiments, targeted heme malignancy NGS panel analyses is used to select iPSC clones which did not accrue hematologic malignancy mutations during reprogramming. For example, targeted heme malignancy NGS panels can focus on myeloid leukemia, lymphoma, and/or other hematologic malignancy-associated genes to generate a smaller, more manageable data set than broader methods. Targeted heme malignancy NGS panel analysis includes the use of highly multiplexed PCR to amplify regions associated with hematologic malignancies followed by next-generation sequencing.

In some embodiments, Droplet Digital PCR (ddPCR) is used to select iPSC clones which did not integrate episomal vectors and that have been passaged enough for episomal vector clearance. As discussed herein, iPSC reprogramming of CD34+ cells can be achieved by delivering episomal vectors encoding reprogramming factors. However, episomal vectors can, albeit rarely, randomly integrate into the cellular genome, which could disrupt developmental processes, homeostasis, etc. Therefore, ddPCR methods can be used to detect residual episomal vector in the iPSC cultures and enable selection of iPSC clones which did not integrate episomal vectors.

In some embodiments, iPSCs which have undergone one or more of these analyses, and have indicated successful reprogramming, are used to build a pre-edited iPSC seed bank.

In some embodiments, gene editing is performed on the pre-edited iPSC seed bank, as described herein. Specifically, in embodiments, the Cas9 and gRNA which target each donor-specific HLA (e.g., HLA-A, HLA-DPB1, and HLA-DQB1) genes are electroporated into the iPSCs, allowing a recovery period of about 1 day to about 1 week. To ensure clonality (e.g., genetic homogeneity due to the cellular population arising from a single modified cell), a single-cell printer can be used to seed single cells into individual wells, for example in a 384-well plate. Such systems can be automated, require minimum-user interface, and ensure proof-of-clonality via imaging of each cell seeded in each well. After expanding the population from a single cell into a cellular colony, the subculture can be further expanded in culture trays with larger surface areas, e.g., into a 96-well plate, 12-well plate, etc., where a portion of the original clonal population of cells can be analyzed via on-target AMP-seq. Such analyses can guide the selection of clones bearing the desired edits. In some embodiments, a portion of cells with each expansion are used for Off-target AMP-seq and/or Translocation AMP-seq analyses to confirm genomic integrity throughout manipulation.

In some embodiments, after assessing that the selected clones are free from genomic aberrations related to the gRNA, the clones can be additionally tested for spontaneous mutations that might arise during expansion. For example, mutations affecting hematologic malignancy genes, indel, translocations, and CNA, e.g., as described for the pre-edited reprogrammed clones. Analyses for spontaneous mutations can include whole-genome sequencing (WGS), KARYOSTAT analysis, Array Comparative Genomic Hybridization (aCGH) analysis, targeted heme malignancy NGS panel AMP-Seq analysis, and/or Droplet Digital PCR (ddPCR).

In some embodiments, clones that are demonstrated to have preserved their genomic integrity are banked as a gene-edited iPSC seed bank.

In some embodiments, the cell line(s) are modified to express cytokines, suicide gene(s), T-cell receptor, single, dual, quad, and/or tandem chimeric antigen receptor (CAR), and/or combinations thereof.

In some embodiments, the cells (e.g., iPSCs or HSCs or progenitors or progenies thereof) of the present disclosure may be modified in a manner such that certain endogenously expressed genes, such as but not limited to genes encoding CCR5 or miR-155, or such that one or more genes encoding cell surface markers including but not limited to CD33, CLL, CD19, CD7, and/or CD38, are deleted or mutated to null their expression or such that they express non-functional proteins or are poorly expressed. Cell surface molecules that can be genetically modified according to this disclosure can be selected from any one of the cell surface molecules known to one of skill in the art, for example from CD1 through CD371, provided that genetic modification of the selected molecule or molecules provide the advantage of eliminating or ameliorating a harmful or toxic function in therapeutical applications of their wildtype counterpart (i.e., unmodified cells).

In various embodiments, the cell line(s) is inserted with CAR specific to myeloma, leukemia or lymphoma targets, including but not limited to CD19, CD33, BCMA, etc.

In various embodiments, the cell line(s) is inserted with tandem CARs, including but not limited CD38/IL3, CD20/CD19, etc.

In various embodiments, the cell line(s) is inserted with disease specific dual CAR, Quad CAR, tandem-CARs, etc.

In various embodiments, the cell line(s) is inserted with leucine-zipper system to incorporate multiple disease-modifying materials, including but not limited to dual CAR, Quad CAR, tandem-CAR, etc.

In various embodiments, the cell line(s) is inserted with leucine-zipper system to incorporate multiple disease-modifying materials.

Non-limiting examples include but are not limited to: (i) cell line(s) deleted for CCR5 to generate CCR5-deleted cellular therapies of HIV-AIDS patients; (2) cell line(s) deleted for CD33 to generate CD33-deleted cellular therapies for treating leukemia and/or lymphoma patients; (3) cell line(s) deleted for CD33 to generate CD33-deleted cellular therapies along with CAR-T, CAR-NK, CAR-T progenitor cells, or CAR-macrophage cells for treating leukemia and/or lymphoma patients.

In various embodiments, the cell line(s) is administered to mitigate the killing of normal cells or adverse effects caused by therapeutical applications of CAR-T therapy with CD33-specific CAR-T cells, with CD7-specific CAR-T cells, with CD8-specific CAR-T cells, with CD19-specific CAR-T cells, with CD20-specific CAR-T cells, with CD22-specific CAR-T cells, with CD123-specific CAR-T cells, with CD125-specific CAR-T cells, with CD133-specific CAR-T cells, with CD371-specific CAR-T cells or any CAR-NK, CAR-T or CAR-macrophage cells targeting the following tumor antigens:
(i) Human epidermal growth factor receptor 2 (HER2)-ovarian cancer, breast cancer, glioblastoma, colon cancer, osteosarcoma, and medulloblastoma;
(ii) Epidermal growth factor receptor (EGFR) positive malignancies, such as—non-small cell lung cancer, epithelial carcinoma, cholangiocarcinoma and glioma;
(iii) Mesothelin-mesothelioma, ovarian cancer, and pancreatic adenocarcinoma;
(iv) Prostate-specific membrane antigen (PSMA)—prostate cancer;
(v) Carcinoembryonic antigen (CEA)—pancreatic adenocarcinoma, breast cancer, and colorectal carcinoma;
(vi) Glypican-3—hepatocellular carcinoma;
(vii) Variant III of the epidermal growth factor receptor (EGFRvIII)—glioblastoma;
(viii) Disialoganglioside 2 (GD2)—neuroblastoma and melanoma;
(ix) Carbonic anhydrase IX (CAIX)—renal cell carcinoma;
(x) Interleukin-13Ra2—glioma;
(xi) Fibroblast activation protein (FAP)—malignant pleural mesothelioma;
(xii) L1 cell adhesion molecule (L1-CAM)—neuroblastoma, melanoma, and ovarian;
(xiii) Cancer antigen 125 (CA 125)—epithelial ovarian cancer;
(xiv) Cluster of differentiation 133 (CD 133)—glioblastoma and cholangiocarcinoma, adenocarcinoma;
(xv) Cancer/testis antigen 1B (CTAG1B)—melanoma and ovarian cancer;
(xvi) Mucin 1—seminal vesicle cancer; and
(xvii) Folate receptor-a (FR-a)—ovarian cancer.
(xviii) EGFRvIII-Glioblastoma.
(xix) Claudin 18.2-solid tumors, advanced gastric adenocarcinoma, pancreatic adenocarcinoma.
(xx) Mesothelin-mesothelioma, metastatic pancreatic, ovarian, cervical, lung.

See, for example, Zhou Z et al., Chimeric antigen receptor T cells applied to solid tumors. Front Immunol. 2022 Oct. 31; or Pooria et al, Novel antigens of CAR T cell therapy: New roads; old destination, Translational Oncology, Volume 14, Issue 7, 2021, each of which is incorporated herein by reference.

Alternatively, the cell populations can be used to generate cell therapies (e.g., HSCs or immune lineages) to be used with FDA approved CAR-T therapy, such as, Tisagenlecleucel, also known as tisa-cel (Kymriah), Axicabtagene ciloleucel, also known as axi-cel (Yescarta), Brexucabtagene autoleucel, also known as brexu-cel (Tecartus), Lisocabtagene maraleucel, also known as liso-cel (Breyanzi), Idecabtagene vicleucel, also known as ide-cel (Abecma), Ciltacabtegene autoleucel, also known as cilta-cel (Carvykti) or any other CAR-T therapy which damage the normal cells during their therapeutic applications.

In some embodiments, the pluripotent cells (e.g., iPSCs or HSCs or progenitors or progenies thereof) of the present disclosure can be further engineered by inserting at least one sequence encoding a transgene operatively linked to an endogenous or exogenous promoter, wherein the transgene is inserted within a genomic safe harbor locus. A genomic safe harbor (GSH) locus refers to a genetic locus that accommodates the insertion of exogenous DNA with either constitutive or conditional expression activity without significantly affecting the viability of somatic cells, progenitor cells, or germ line cells and ontogeny. Well known safe harbor loci include the AAVS1 adeno-associated virus insertion site on chromosome 19, the human homolog of the murine Rosa26 locus, and the CCR5 chemokine receptor gene. Tools and techniques for the insertion of transgene (i.e., the exogenous DNA) into safe harbor locus are well known to one of skill in the art, see for example Papapetrou E P et al. Gene Insertion Into Genomic Safe Harbors for Human Gene Therapy. Mol Ther. (2016) 678-84.

According to aspects and embodiments of this disclosure, a method is provided for making an HLA-modified cell of the disclosure using CRISPR-Cas9 gene editing. Exemplary sgRNAs are disclosed herein that can be used singly, or in some embodiments in combination to produce a plurality of edits (e.g., double strand breaks) in the target gene. The exemplary sgRNA are disclosed herein for generating deletions in exon 1 and/or exon 2 of various HLA genes (including within genomic coordinates shown in FIG. 8B). In the various embodiments, the method comprises contacting a cell with a Cas9 endonuclease (which can be delivered to the cell using any of the known processes) and one or more gRNAs (e.g., sgRNAs) targeting the Cas9 endonuclease to the HLA-specific or HLA allele-specific regions.

Table 2 lists spacer sequences useful for targeting HLA alleles as indicated, which can be incorporated into a gRNA (e.g., sgRNA) of a CRISPR-Cas9 system. In some embodiments, a sgRNA further comprises a scaffold sequence fused to the 3′ end of a spacer sequence. In some embodiments, a gRNA further comprises a tracr mate sequence fused to the 3′ end of a spacer sequence.

In some embodiments, the cell is homozygous for HLA-A*01:01, and the cell is contacted with sgRNAs comprising a nucleotide sequence selected from Table 2. In some embodiments, the sgRNA comprises the nucleotide sequence:

(SEQ ID NO: 6)
GAGGGTTCGGGGCGCCATGA.

In some embodiments, the cell is homozygous for HLA-DQB1*02:01, and the cell is contacted with sgRNAs comprising a nucleotide sequence selected from Table 2. In some embodiments, the sgRNA comprises the nucleotide sequence:

(SEQ ID NO: 26)
GTGCTACTTCACCAACGGGA
or
(SEQ ID NO: 27)
AGGTCGTGCGGAGCTCCAAC.

In some embodiments, the cell is homozygous or heterozygous for HLA-DPB1*01:01 or DPB1*04:01, and the cell is contacted a sgRNA comprising a nucleotide sequence selected from Table 2. In some embodiments, the sgRNA comprises the nucleotide sequence:

(SEQ ID NO: 21)
GGAGAGATACATCTACAACC.

In some embodiments, hiPSCs are used to generate embryoid bodies (EB), which can be used for generation of (i.e., isolation or enrichment of) CD34+ cells. For example, EBs can be dissociated, and the CD34+ hematopoietic precursors isolated or enriched. In some embodiments, human iPSC aggregates are expanded in a bioreactor as described, for example, in Abecasis B. et al., Expansion of 3D human induced pluripotent stem cell aggregates in bioreactors: Bioprocess intensification and scaling-up approaches. J. of Biotechnol. 246 (2017) 81-93.

Other bioreactors could include, but are not limited to, shear stress, mechanical strain and pulsed electromagnetic field bioreactors, large-scale stirred tank bioreactors, automated bioreactors, rotating wall bioreactors (RWBs), and rocking motions as seen with wave bioreactors, organ-on-chip bioreactors. Other bioreactor configurations can be employed that enable continuous, perfusion operation such as packed bed bioreactors (PBBs), fluidized bed bioreactors (FBBs), or PBBs and/or FBBs including the use of microcarriers, CultiBag bioreactors, and membrane bioreactors such as hollow fiber bioreactors (HFBs). Such bioreactors are contemplated for generating the pluripotent cells or progenitors or progenies derived therefrom of the present disclosure. Operation of the bioreactors may require coupling with an internal or external cell retention device on a recycle line, by centrifugation, sedimentation, ultrasonic separation or microfiltration with spin-filters, alternating tangential flow (ATF) filtration or tangential flow filtration (TFF).

In some embodiments, the process of generating cell populations comprising HSCs and/or HSPCs or progeny thereof can comprise generating CD34+-enriched cells from the differentiated pluripotent stem cells (e.g., EBs) and inducing endothelial-to-hematopoietic differentiation (e.g., for at least 2 days, but no more than 12 days). In some embodiments, HSCs comprising relatively high frequency of LT-HSCs can be generated from the cell populations using various stimuli or factors, including mechanical, biochemical, metabolic, and/or topographical stimuli, as well as factors such as extracellular matrix, niche factors, cell-extrinsic factors, induction of cell-intrinsic properties; and including pharmacological and/or genetic means.

In some embodiments, the method comprises preparing endothelial cells with hemogenic potential from pluripotent stem cells, prior to induction of EHT. In some embodiments, the combined over-expression of GATA2/ETV2, GATA2/TAL1, or ER71/GATA2/SCL can lead to the formation of endothelial cells with hemogenic potential from PSC sources. In some embodiments, the method comprises overexpression of E26 transformation-specific variant 2 (ETV2) transcription factor in the iPSCs. Following CD34+ enrichment, HSCs are then generated from the endothelial cells using mechanical, biochemical, pharmacological and/or genetic stimulation or modification. ETV2 can be expressed by introduction of an encoding non-integrating episomal plasmid, for constitutive or inducible expression of ETV2, and for production of transgene-free hemogenic ECs. In some embodiments, ETV2 is expressed from an mRNA introduced into the iPSCs. mRNA can be introduced using any available method, including electroporation or lipofection. Differentiation of cells expressing ETV2 can comprise addition of VEGF-A. See, Wang K, et al., Robust differentiation of human pluripotent stem cells into endothelial cells via temporal modulation of ETV2 with mRNA. Sci. Adv. Vol. 6 (2020). Cells generated in this manner may be used for producing CD34+ cells and inducing EHT according to embodiments of this disclosure. See PCT/US2021/062884, which is hereby incorporated by reference in its entirety.

In some embodiments, iPSC differentiation proceeds until cells are at least about 10% CD34+, or at least about 20% CD34+, or at least about 25% CD34+, or at least about 30% CD34+, or at least 40% CD34+. In some embodiments, CD34+ enrichment and EHT may be induced at Day 6 to Day 14 of iPSC differentiation, such as for example, Day 7, Day 8, Day 9, Day 10, Day 11, Day 12, Day 13, or Day 14. Differentiation of iPSCs can be according to known techniques. In some embodiments, iPSC differentiation involves factors such as, but not limited to, combinations of bFGF, Y27632, BMP4, VEGF, SCF, EPO, TPO, IL-6, IL-11, and/or IGF-1. In some embodiments, hPSCs are differentiated using feeder-free, serum-free, and/or GMP-compatible materials. In some embodiments, hPSCs are co-cultured with murine bone marrow-derived feeder cells such as OP9 or MS5 or mouse embryonic fibroblast cell line in serum-containing medium. The culture can contain growth factors and cytokines to support differentiation of embryoid bodies or monolayer system. The OP9 co-culture system can be used to generate multipotent HSPCs, which can be differentiated further to several hematopoietic lineages including T lymphocytes, B lymphocytes, megakaryocytes, monocytes or macrophages, and erythrocytes. See Netsrithong R. et al., Multilineage differentiation potential of hematoendothelial progenitors derived from human induced pluripotent stem cells, Stem Cell Research & Therapy Vol. 11 Art. 481 (2020). Alternatively, a step-wise process using defined conditions with specific signals can be used. For example, the expression of HOXA9, ERG, RORA, SOX4, and MYB in human PSCs favors the direct differentiation into CD34+/CD45+ progenitors with multilineage potential. Further, expression of factors such as HOXB4, CDX4, SCL/TAL1, or RUNX1a support the hematopoietic program in human PSCs. See Doulatov S. et al., Induction of multipotential hematopoietic progenitors from human pluripotent stem cells via re-specification of lineage-restricted precursors, Cell Stem Cell. 2013 Oct. 3; 13 (4).

Induction of EHT can be with any known process. In some embodiments, induction of EHT generates an HSC population comprising LT-HSCs. In some embodiments, EHT generates a cell population comprising HSPCs. In some embodiments, EHT generates HSCs and/or HSPCs through endothelial or hemogenic endothelial cell (HEC) precursors using mechanical, biochemical, pharmacological and/or genetic means (e.g., via stimulation, inhibition, and/or genetic modifications). In some embodiments, the EHT generates a stem cell population comprising one or more of long-term hematopoietic stem cells (LT-HSCs), short-term hematopoietic stem cells (ST-HSCs), and HSPCs. In various embodiments, EHT can be induced in the culture for from 2 days to 12 days, such as about 4 days to about 8 days (e.g., about 4 days, about 5 days, about 6 days, about 7 days, or about 8 days). In some embodiments, EHT is induced in the culture from about 5 days to about 7 days.

In some embodiments, the HSC and/or HSPC population or fraction thereof is differentiated to T cells or progenitors or derivatives thereof independent of the use of an agonist of a mechanosensitive receptor or a mechanosensitive channel, such as Yoda1. In some embodiments, the use of an agonist of a mechanosensitive receptor or a mechanosensitive channel (e.g., Yoda1) is optional. Thus, in some embodiments, CD34+ cells are enriched from a differentiated pluripotent stem cell population to prepare a CD34+-enriched population. Endothelial-to-hematopoietic transition of the CD34+-enriched cell population is induced for at least two days, but no more than 12 days in which the use of an agonist of a mechanosensitive receptor or a mechanosensitive channel such as Yoda1, jedi1, jedi2, or ssRNA40 is optional. The HSCs and/or HSPCs are differentiated to a progenitor T cell population or a T cell population (e.g., as described herein). In some embodiments, the endothelial-to-hematopoietic transition of the CD34+-enriched cell population is induced for at least for two days, and further for about 4 hours, or about 8 hours, or about 12 hours, or about 16 hours, or about 20 hours, or about 24 hours, or about 2 days, or about 3 days, or about 4 days, or about 5 days, or about 6 days, or about 7 days, or about 8 days, or about 9 days, or about 10 days. The total EHT differentiation proceeds for no more than 12 days.

In various embodiments, CD34+ cells (e.g., the floater and/or adherent cells) are harvested from the culture undergoing endothelial-to-hematopoietic transition, such as between Day 10 to Day 20 of iPSC differentiation, such as from Day 10 to Day 17, or from Day 12 to Day 15 of iPSC differentiation.

In some embodiments, the method comprises increasing the expression or activity of dnmt3b in PSCs, embryoid bodies, CD34-enriched cells, ECs, HECs or HSCs, which can be by mechanical, genetic, biochemical, or pharmacological means. In some embodiments, the method comprises increasing activity or expression of DNA (cytosine-5-)-methyltransferase 3 beta (Dnmt3b) and/or GTPase IMAP Family Member 6 (Gimap6) in the cells. See WO 2019/236943 and WO 2021/119061, which is hereby incorporated by reference in its entirety. In some embodiments, the induction of EHT comprises increasing the expression or activity of dnmt3b.

In some embodiments, cells are contacted with an effective amount of an agent such as but limited to an agonist of a mechanosensitive receptor or a mechanosensitive channel that increases the activity or expression of Dnmt3b. In some embodiments, the mechanosensitive receptor is Piezo1. An exemplary Piezo1 agonist is Yoda1.

In some embodiments, the mechanosensitive receptor is Trpv4. An exemplary Trpv4 agonist is GSK1016790A. Yoda1 (2-[5-[[(2,6-Dichlorophenyl)methyl]thio]-1,3,4-thiadiazol-2-yl]-pyrazine) is a small molecule agonist developed for the mechanosensitive ion channel Piezol. Syeda R, Chemical activation of the mechanotransduction channel Piezo1. eLife (2015).

Derivatives of Yoda1 can be employed in various embodiments. For example, derivatives comprising a 2,6-dichlorophenyl core are employed in some embodiments. Exemplary agonists are disclosed in Evans E L, et al., Yoda1 analogue (Dooku1) which antagonizes Yoda1-evoked activation of Piezo1 and aortic relaxation, British J. of Pharmacology 175 (1744-1759): 2018. Still other Piezo1 agonist include Jedi1, Jedi2, single-stranded (ss) RNA (e.g., ssRNA40) and derivatives and analogues thereof. See Wang Y., et al., A lever-like transduction pathway for long-distance chemical-and mechano-gating of the mechanosensitive Piezo1 channel. Nature Communications (2018) 9:1300; Sugisawa, et al., RNA Sensing by Gut Piezo1 Is Essential for Systemic Serotonin Synthesis, Cell, Volume 182, Issue 3, 2020, Pages 609-624, which are hereby incorporated by reference in their entireties. These Piezo1 agonists are commercially available. In various embodiments, the effective amount of the Piezo1 agonist or derivative is in the range of about 1 μM to about 500 μM, or about 5 μM to about 200 μM, or about 5 μM to about 100 μM, or in some embodiments, in the range of about 25 μM to about 150 μM, or about 25 μM to about 100 μM, or about 25 μM to about 50 μM. Alternatively, single-stranded (ss) RNA (e.g., ssRNA40), and derivatives and analogues thereof, can be used for Piezo1 activation.

In various embodiments, pharmacological Piezo1 activation is applied to CD34+ cells (i.e., CD34-enriched cells). In certain embodiments, pharmacological Piezo1 activation may further be applied to iPSCs, embryoid bodies, ECs, hemogenic endothelial cells (HECs), HSCs, hematopoietic progenitors, as well as hematopoietic lineage(s). In certain embodiments, Piezo1 activation is applied at least to EBs generated from iPSCs, CD34+ cells isolated from EBs, and/or combinations thereof. The use of Piezo1 activation for generation of HSCs or progeny thereof is described in US 2021/0222125 and US 2022/00049221, which are hereby incorporated by reference in their entireties.

Alternatively, or in addition, the activity or expression of Dnmt3b can be increased directly in the cells, e.g., in CD34-enriched cells. For example, mRNA expression of Dnmt3b can be increased by delivering Dnmt3b-encoding transcripts to the cells, or by introducing a Dnmt3b-encoding transgene, or a transgene-free method, not limited to introducing a non-integrating episome to the cells. In some embodiments, gene editing is employed to introduce a genetic modification to Dnmt3b expression elements in the cells, such as, but not limited to, to increase promoter strength, ribosome binding, RNA stability, and/or impact RNA splicing.

In embodiments of this disclosure employing mRNA delivery to cells, known chemical modifications can be used to avoid the innate-immune response in the cells. For example, synthetic RNA comprising only canonical nucleotides can bind to pattern recognition receptors, and can trigger a potent immune response in cells. This response can result in translation block, the secretion of inflammatory cytokines, and cell death. RNA comprising certain non-canonical nucleotides can evade detection by the innate immune system, and can be translated at high efficiency into protein. See U.S. Pat. No. 9,181,319, which is hereby incorporated by reference, particularly with regard to nucleotide modification to avoid an innate immune response.

In some embodiments, expression of Dnmt3b is increased by introducing a transgene into the cells, which can direct a desired level of overexpression (with various promoter strengths or other selection of expression control elements). Transgenes can be introduced using various viral vectors or transfection reagents (including Lipid Nanoparticles) as are known in the art. In some embodiments, expression of Dnmt3b is increased by a transgene-free method (e.g., episome delivery or lipid nanoparticle with mRNA). In some embodiments, expression or activity of Dnmt3b or other genes disclosed herein are increased using a gene editing technology, for example, to introduce one or more modifications to increase promoter strength, ribosome binding, or RNA stability.

In some embodiments, the method comprises applying cyclic 2D, 3D, or 4D stretch to cells. In various embodiments, the cells subjected to cyclic 2D, 3D, or 4D stretch are selected from one or more of CD34-enriched cells, iPSCs, ECs, and HECs. For example, a cell population is introduced to a bioreactor that provides a cyclic strain, or a biomechanical stretching or a cyclic-strain biomechanical stretching. Cyclic-strain biomechanical stretching is described in WO 2017/096215, which is hereby incorporated by reference in its entirety.

In some embodiments, cells are contacted with an effective amount of an agent that (a) modulates histone acetylation; or (b) modulates histone methylation; or (c) modulates TGF beta signaling; or (d) modulates wnt and/or notch signaling pathway.

Modulating agents can be selected from inhibitors which modulate signaling through TGF beta pathway, wnt pathway, notch pathway or modulate histone methylation and/or acetylation. Some of the proteins that are known to be acetylated include p53, HSP90, tubulin, NF-κB, HIF-1a, RUNX3, STAT-3, E2F1, Ku70 and c-MYC. Acetylation functions as a broad post-translational modification regulating protein functions including DNA-binding, activity of transcription factors, subcellular localization, and protein stability.

Nonlimiting examples of modulating agents include but are not limited to inhibitor of histone methyltransferase EZH1, DNA methyltransferase inhibitors (DNMTi), histone deacetylase inhibitors (HDACi), Suberoylanilide bis-hydroxamic acid (SBHA), Tranylcypromine, LSD1 inhibitors, such as IV RN-1, LSD1-C76, LSD1 inhibitor II S2101, LSD1 inhibitor III CBB1007, LSD1 inhibitor I, SNDX-275 (MS-275, Entinostat), CI-994 (Tacedinaline), MGCD-0103, Valproic acid (VPA), Sodium butyrate, Phenyl butyrate (S-HDAC-42, AR-42), Depsipeptide (Romidepsin), Apicidin, JNJ-26481585, Suberoylanilide hydroxamic acid (SAHA; Vorinostat), NVP-LAQ824 (Dacinostat), CR-2408, RAS2410 (Resminostat), Trichostatin-A (TSA), LBH589 (Panobinostat), ITF2357 (Gavinostat), PXD101 (Belinostat), ACY-1215 (Rocilinostat), KD5170, and Tubacin.

Several TGF-β R kinase inhibitors have been designed to bind the ATP-binding domain of TGF-β R kinase and inhibit ATP kinase activity and block the downstream signaling cascade. TGF-β inhibitors can be selected from one or more of Galunisertib (LY21557299), ALK5 inhibitor II (E-616452), LY364947, A83-01, and DMH1, LY573636 (Tasisulam), LY2109761, LY364937, Ki26894, LY580276, SB-431542, SB-505124, A83-01, SD-093 and SD-208, IN-1130, and Vactosertib (TEW-7197). TGF-β inhibitors could also include antibodies, such as but not limited to SRK181-mlgG1, Fresolimumab, LY3022859, 264RAD, 1D11, 2G7, or a pyrimidoindole derivative including, for example, UM171 or UM729.

In certain cases, the agent includes a compound that inhibits a protein that propagates p38 signaling, such as SB203580. In additional embodiments, the one or more agents include a compound that inhibits a protein that promotes beta-catenin degradation selected from one or more of lithium chloride, CHIR99021, ICG-001, XAV939, pyrvinium, BIO, C2 inhibitor, CRT-3, -5 and -14, stapled peptide StAx35R, and FGF2 or a recombinant version thereof.

In some embodiments the method employs an allosteric agonist, including but not limited to yoda 1, jedi 1, jedi 2, docosahexaenoic acid or analogs thereof or any agonist that modulates the activity of the mechanosensitive Piezo channels.

In a non-limiting example, the iPSC cell line(s)-derived HSCs are generated by CD34-enrichment from embryonic bodies and endothelial-to-hematopoietic transition that is induced at Day 8 to Day 15 of iPSC differentiation. CD34+ cells are harvested from culture undergoing endothelial-to-hematopoietic transition, including harvesting of CD34+ floater and/or adherent cells. In some embodiments, the induction of endothelial-to-hematopoietic transition comprises increasing the expression or activity of dnmt3b as described.

It is contemplated that the one or more agents can be added to act concomitantly or to act on the same or different pathways. For example, they could simultaneously act as inhibitors of TGF-beta or they can act independently to inhibit histone demethylase and TGF-beta respectively, which could be simultaneous or sequential.

Where cell populations or banks are described herein as having a certain phenotype it is understood that the phenotype represents a significant portion of the cell population, such as at least 25%, at least 40%, or at least about 50%, or at least about 60%, or at least about 75%, or at least about 80%, or at least about 90% of the cell population. Further, at various steps, cell populations can be enriched for cells of a desired phenotype, and/or depleted of cells of an undesired phenotype, such that cell population comprise at least about 75%, or at least about 80%, or at least about 90% of the desired phenotype. Such positive and negative selection methods are known in the art. For example, cells can be sorted based on cell surface antigens (including those described herein) using a fluorescence activated cell sorter, or magnetic beads which bind cells with certain cell surface antigens. Negative selection columns can be used to remove cells expressing undesired cell-surface markers. In some embodiments, cells are enriched for CD34+ cells (prior to and/or after undergoing EHT). In some embodiments, the cell population is cultured under conditions that promote expansion of CD34+ cells to thereby produce an expanded population of stem cells.

In various embodiments, CD34+ cells (e.g., the floater and/or adherent cells) are harvested from the culture undergoing endothelial-to-hematopoietic transition between Day 8 to Day 15 of iPSC differentiation.

In various embodiments, the HSCs or CD34-enriched cells are further expanded. For example, the HSCs or CD34-enriched cells can be expanded according to methods disclosed in U.S. Pat. Nos. 8,168,428; 9,028,811; 10,272,110; and 10,278,990, which are hereby incorporated by reference in their entireties. In some embodiments, ex vivo expansion of HSCs or CD34-enriched cells employs prostaglandin E2 (PGE2) or a PGE₂ derivative. In some embodiments of this disclosure, the HSCs comprise at least about 0.01% LT-HSCs, or at least about 0.05% LT-HSCs, or at least about 0.1% LT-HSCs, or at least about 0.5% LT-HSCs, or at least about 1% LT-HSCs.

Hematopoietic stem cells (HSCs) which give rise to erythroid, myeloid, and lymphoid lineages, can be identified based on the expression of CD34 and the absence of lineage specific markers (termed Lin−). In some embodiments, a population of stem cells comprising HSCs are enriched, for example, as described in U.S. Pat. No. 9,834,754, which is hereby incorporated by reference in its entirety. For example, this process can comprise sorting a cell population based on expression of one or more of CD34, CD90, CD45, CD38, and CD43. A fraction can be selected for further differentiation that is one or more of CD34⁺, CD90⁺ and/or CD45+, CD38⁻, and CD43⁻. In some embodiments, the stem cell population for differentiation to a hematopoietic lineage is at least about 80% CD34⁺, or at least about 90% CD34⁺, or at least about 95% CD34⁺.

In some embodiments, multi-potent hematopoietic stem cells (HSCs) self-renew and differentiate into two types of progenitor cells with specific lineage commitments. Like HSCs, human lineage-restricted progenitor cells also express CD34 and Flt-3/Flk-2. Myeloid progenitors (MPs), in human and mouse, express IL-3 R alpha and give rise to cells of the myeloid lineage including megakaryocytes, erythrocytes, granulocytes, and macrophages. Lymphoid progenitors (LPs) are cells that develop from HSCs and give rise to B cells, T cells, and Natural Killer (NK) cells. Human bone marrow LPs are CD34+CD38+Neprilysin+, and cord blood CLPs are CD34+CD38−CD7+.

In some embodiments, the cell populations are differentiated to hematopoietic lineage cells for administration to a recipient. In various embodiments, the hematopoietic lineage cells are selected from common lymphoid precursor (CLP) cells, granulocyte-monocyte progenitor (GMP) cells, progenitor-T cells, T lymphocytes, B lymphocytes, Natural Killer cells, neutrophils, monocyte, macrophages, red cells, megakaryocytes, and platelets. In some aspects and embodiments, the disclosure provides a method for generating a CD7+ progenitor T cell population, or a derivative of this population. For example, the method comprises generating a hematopoietic stem cell (HSC) population comprising human long-term hematopoietic stem cells (LT-HSCs) from iPSCs (e.g., hiPSCs). The HSC population is derived by induction of endothelial-to-hematopoietic transition of CD34+ cells (e.g., CD34+ cells derived from embryoid bodies). The HSC population (or cells isolated therefrom) is cultured with a partial or full Notch ligand, sonic hedgehog (SHH), RetroNectin (or other extracellular matrix component(s)), and/or combinations thereof, to produce a population comprising CD7+ progenitor T cells or a derivative cell population.

The Notch signaling pathway regulates the formation, differentiation, and function of progenitor T-cells, pre-T cells, and/or mature T lymphocytes. In vivo, T cell development proceeds after lymphocyte progenitors differentiate from bone marrow hematopoietic stem cells and migrate to the thymus. Specialized thymic epithelial cells induce T cells to develop along a controlled pathway. Notch signaling plays a critical role during T lineage commitment in the thymus. As lymphoid progenitors enter the thymus, they encounter dense expression of Notch ligands on thymic epithelium that drives thymopoiesis. The present disclosure provides HSC populations generated ex vivo from iPSCs and which respond to Notch ligand, SHH, and/or component(s) of extracellular matrix, by robust production of T progenitor cells and T cell lineages ex vivo.

In some embodiments, the stem cell population, or CD34-enriched cells or fraction thereof, or derivative population are expanded as described in US 2020/0308540, which is hereby incorporated by reference in its entirety. For example, the cells are expanded by exposing the cells to an aryl hydrocarbon receptor antagonist including, for example, SR1 or an SR1-derivative. See also, Wagner et al., Cell Stem Cell 2016; 18 (1): 144-55 and Boitano A., et al., Aryl Hydrocarbon Receptor Antagonists Promote the Expansion of Human Hematopoietic Stem Cells. Science 2010 Sep. 10; 329 (5997): 1345-1348.

In some embodiments, the compound that promotes expansion of CD34⁺ cells includes a pyrimidoindole derivative including, for example, UM171 or UM729 (see US 2020/0308540, which is hereby incorporated by reference).

In some embodiments, the stem cell population or CD34-enriched cells are further enriched for cells that express Periostin and/or Platelet Derived Growth Factor Receptor Alpha (pdgfra) or are modified to express Periostin and/or pdgfra, as described in WO 2020/205969 (which is hereby incorporated by reference in its entirety). Such expression can be by delivering encoding transcripts to the cells, or by introducing an encoding transgene, or a transgene-free method, not limited to introducing a non-integrating episome to the cells. In some embodiments, gene editing is employed to introduce a genetic modification to expression elements in the cells, such as to modify promoter activity or strength, ribosome binding, RNA stability, or impact RNA splicing.

In still other embodiments, the stem cell population or CD34-enriched cells are cultured with an inhibitor of histone methyltransferase EZH1. Alternatively, EZH1 is partially or completely deleted or inactivated or is transiently silenced in the stem cell population. Inhibition of EZH1 can direct myeloid progenitor cells (e.g., CD34+CD45+) to lymphoid lineages. See WO 2018/048828, which is hereby incorporated by reference in its entirety. In still other embodiments, EZH1 is overexpressed in the stem cell population.

In various embodiments, the cell lines developed from primary cells or the cell lines derived from iPSC cell line or banks thereof are hematopoietic lineage cells. The hematopoietic lineage is selected from common lymphoid precursor (CLP) cells, granulocyte-monocyte progenitor (GMP) cells, progenitor-T cells, T lymphocytes, B lymphocytes, Natural Killer cells, neutrophils, monocyte, macrophages, red cells, megakaryocytes, and platelets.

In some embodiments, the HSC population or fraction thereof is differentiated ex vivo to progenitor T cells, T cells, NK cells, and/or fractions or analogous thereof.

In some embodiments, the HSC population or fraction thereof is cultured with a partial or full Notch ligand to produce a population comprising CD7+ progenitor T cells or a derivative cell population.

In some embodiments, the derivative cell population is a T cell, precursors, subtypes, and derivatives of T cells, or NK cell population.

In some embodiments, the cell population is cultured with a Notch ligand, partial or full, SHH, extracellular matrix component(s), and/or combinations thereof, ex vivo, to differentiate HSCs to CD7⁺ progenitor T cells, and optionally to a T cell lineage or other lineage (e.g., NK cell). Further, according to known processes, xenogenic OP9-DL1 cells are often employed for differentiation to T cells. The OP9-DL1 co-culture system uses a bone marrow stromal cell line (OP9) transduced with the Notch ligand delta-like-1 (DLL1) to support T cell development from stem cell sources. The OP9-DL1 system limits the potential of the cells for clinical application. There is a need for feeder-cell-free systems that can generate T lymphocytes from hiPSCs for clinical use, and in some embodiments the present disclosure meets this objective.

The term “Notch ligand” as used herein refers to a ligand capable of binding to a Notch receptor polypeptide present in the membrane of a hematopoietic stem cell or progenitor T cell. The Notch receptors include Notch-1, Notch-2, Notch-3, and Notch-4. Notch ligands typically have a DSL domain (D-Delta, S-Serrate, and L-Lag2) comprising 20-22 amino acids at the amino terminus, and from 3 to 8 EGF repeats on the extracellular surface. In various embodiments, the Notch ligand comprises at least one of Delta-Like-1 (DLL1), Delta-Like-4 (DLL4), SFIP3, and DeltaMax (disclosed in PCT/US2020/041765 and PCT/US2020/030977, which are hereby incorporated by reference in their entirety) or a functional portion thereof. A key signal that is delivered to incoming lymphocyte progenitors by the thymus stromal cells in vivo is mediated by DL4, which is expressed by cortical thymic epithelial cells.

The earliest intrathymic progenitors express high levels of CD34 and CD7, do not express CD1a, and are triple-negative (TN) for mature T cell markers: CD4, CD8, and CD3. Commitment to the T cell lineage is associated with the expression of CD1a by CD7-expressing pro-thymocytes. Thus, immature stages of T-cell development are typically delineated as CD34⁺CD1a⁻ (most immature) and CD34⁺CD1a⁺ cells. The transition from CD34⁺CD7⁺CD1a⁻ to CD34⁺CD7⁺CD1a⁺ by early thymocytes is associated with T-cell commitment. CD34⁺CD7⁺CD1a⁺ cells are likely T-lineage restricted. Following this stage, thymocytes progress to a CD4 immature single positive stage, at which point CD4 is expressed in the absence of CD8. Thereafter, a subset of the cells differentiates to the CD4⁺CD8⁺ double positive (DP) stage. Finally, following TCRα rearrangement, TCRαβ-expressing DP thymocytes undergo positive and negative selection, and yield CD4⁺CD8⁻ and CD4⁻CD8⁺ single positive (SP) T-cells.

In some embodiments, progenitor T cells are isolated by enrichment for CD7 expression. In some embodiments, progenitor T cells are expanded as described in US 2020/0308540, which is hereby incorporated by reference in its entirety. For example, the cells may be expanded by exposing the cells to an aryl hydrocarbon receptor antagonist including, for example, SR1 or an SR1-derivative. See also, Wagner et al., Cell Stem Cell 2016; 18 (1): 144-55. In some embodiments, the compound that promotes expansion includes a pyrimidoindole derivative including, for example, UM171 or UM729 (see US 2020/0308540, which is hereby incorporated by reference).

Differentiation to progenitor T cells can further include in some embodiments the presence of stem cell factor (SCF), Flt3L and interleukin (IL)-7. In various embodiments, CD7+ progenitor T cells created express CD1a. The CD7+ progenitor T cells do not express CD34 or express a diminished level of CD34 compared to the HSC population. In some embodiments, the CD7+ progenitor T cells (or a portion thereof) further express CD5. Accordingly, the phenotype of the progenitor T cells may be CD7⁺CD1a⁺. In some embodiments, the phenotype of the progenitor T cells is CD7⁺CD5⁺. In some embodiments, the progenitor T cells are CD7⁺CD1a⁺CD5⁺, and optionally CD34⁺. In some embodiments, the progenitor T cells are CD7⁺CD1a⁻CD5, and optionally CD34⁺.

In some embodiments, the progenitor T cells exhibit a diminished level of CD34 expression, minimal CD34 expression (compared to the HSC population), or no CD34 expression. In some embodiments, CD34 expression is diminished in the population by at least about 50%, or at least about 75%, relative to the HSC population.

In some embodiments, the Notch ligand is an anti-Notch (agonistic) antibody that can bind and engage Notch signaling. In some embodiments, the antibody is a monoclonal antibody (including a human or humanized antibody), a single chain antibody (scFv), a nanobody, or other antibody fragment or antigen-binding molecule capable of activating the Notch signaling pathway.

In some embodiments, the Notch ligand is a Delta family Notch ligand. The Delta family ligand in some embodiments is Delta-1 (Genbank Accession No. AF003522, Homo sapiens), Delta-like 1 (DLL1, Genbank Accession No. NM_005618 and NP_005609, Homo sapiens; Genbank Accession No. X80903, 148324, M. musculus), Delta-4 (Genbank Accession No. AF273454, BAB18580, Mus musculus; Genbank Accession No. AF279305, AAF81912, Homo sapiens), and/or Delta-like 4 (DLL4; Genbank Accession. No. Q9NR61, AAF76427, AF253468, NM_019074, Homo sapiens; Genbank Accession No. NM 019454, Mus musculus). Notch ligands are commercially available or can be produced, for example, by recombinant DNA techniques.

In some embodiments, the Notch ligand comprises an amino acid sequence that is at least about 70%, or at least about 80%, or at least about 90%, or at least about 95%, or at least about 97% identical (e.g., about 100% identical) to human DLL1 or DLL4 Notch ligand. Functional derivatives of Notch ligands (including fragments or portions thereof) will be capable of binding to and activating a Notch receptor. Binding to a Notch receptor may be determined by a variety of methods known in the art including in vitro binding assays and receptor activation/cell signaling assays.

In some embodiments, the Notch ligand is a DLL4 having one or more affinity enhancing mutations, such as one or more (or all) of: G28S, F107L, 1143F, H194Y, L206P, N257P, T271L, F280Y, S301R and Q305P, with respect to hDLL4. See Gonzalez-Perez, et al., Affinity-matured DLL4 ligands as broad-spectrum modulators of Notch signaling, Nature Chemical Biology (2022).

In various embodiments, the Notch ligands are soluble, and are optionally immobilized on microparticles or nanoparticles, which are optionally paramagnetic to allow for magnetic enrichment or concentration processes. In still other embodiments, the Notch ligands are immobilized on a 2D or 3D culture surface, optionally with other adhesion molecules such as VCAM-1. See US 2020/0399599, which is hereby incorporated by reference in its entirety. In other embodiments, the beads or particles are polymeric (e.g., polystyrene or PLGA), gold, iron dextran, or constructed of biological materials, such as particles formed from lipids and/or proteins. In various embodiments, the particle has a diameter or largest dimension of from about 0.01 μm (10 nm) to about 500 μm (e.g., from about 1 μm to about 7 μm). In still other embodiments, polymeric scaffolds with conjugated ligands can be employed, as described in WO 2020/131582, which is hereby incorporated by reference in its entirety. For example, scaffold can be constructed of polylactic acid, polyglycolic acid, PLGA, alginate or an alginate derivative, gelatin, collagen, agarose, hyaluronic acid, poly(lysine), polyhydroxybutyrate, poly-epsilon-caprolactone, polyphosphazines, poly(vinyl alcohol), poly(alkylene oxide), poly(ethylene oxide), poly(allylamine), poly(acrylate), poly(4-aminomethylstyrene), pluronic polyol, polyoxamer, poly(uronic acid), poly(anhydride), poly(vinylpyrrolidone), and any combination thereof. In some embodiments, the scaffold comprises pores having a diameter between about 1 μm and 100 μm.

In some embodiments, the C-terminus of the Notch ligand is conjugated to the selected support. In some embodiments, this can include adding a sequence at the C-terminal end of the Notch ligand that can be enzymatically conjugated to the support, for example, through a biotin molecule. In another embodiment, a Notch ligand-Fc fusion is prepared, such that the Fc segment can be immobilized by binding to protein A or protein G that is conjugated to the support. Of course, any of the known protein conjugation methods can be employed.

Thus, in various embodiments, the Notch ligand is immobilized, functionalized, and/or embedded in 2D or 3D culture system. The Notch ligand may be incorporated along with a component of extracellular matrix, such as one or more selected from fibronectin, RetroNectin, and laminin. In some embodiments, the Notch ligand and/or component of extracellular matrix are embedded in inert materials providing 3D culture conditions. Exemplary materials include, but are not limited to, cellulose, alginate, and combinations thereof. In some embodiments, the Notch ligand, a component of extracellular matrix, or combinations thereof, are in contact with culture conditions providing topographical patterns and/or textures (e.g., roughness) to cells conducive to differentiation and/or expansion.

In some embodiments, cell populations or banks are differentiated to progenitor T cells by culture in medium comprising TNF-α and/or antagonist of aryl hydrocarbon/dioxin receptor (SR1), and in the presence of Notch ligand. See US 2020/0390817, US 2021/0169934, and US 2021/0169935, which are hereby incorporated by reference in its entirety. In some embodiments the HSCs are cultured in a medium comprising TNF-α, IL-7, thrombopoietin (TPO), Flt3L, and stem cell factor (SCF), and optionally SR1, in the presence of an immobilized Delta-Like-4 ligand and a fibronectin fragment. In some embodiments, the cells are cultured with RetroNectin, which is a recombinant human fibronectin containing three functional domains: the human fibronectin cell-binding domain (C-domain), heparin-binding domain (H-domain), and CS-1 sequence domain. In some embodiments, cells are cultured in the presence of an immobilized Delta-Like-4 ligand and a RetroNectin. In some embodiments, cells are cultured in the presence of an immobilized Delta-Like-4 ligand, TNF-alpha, and a RetroNectin. In some embodiments, cells are cultured in the presence of an immobilized Delta-Like-1 ligand and a RetroNectin. In some embodiments, cells are cultured in the presence of SFIP3 and RetroNectin. In some embodiments, cells are cultured in the presence of an immobilized Delta-Like-4 ligand and SHH molecules and/or functional derivatives thereof. Exemplary fibronectin fragments include one or more RGDS, CS-1, and heparin-binding motifs. Fibronectin fragments can be free in solution or immobilized to the culture surface or on particles. In some embodiments, cells are cultured for 5 to 7 days to prepare CD7+ progenitor T cells.

In various embodiments, the method produces progenitor T cells, or a T cell lineage, by culturing the HSC population with the Notch ligand (including any of the embodiments described above) with or without component(s) extracellular matrix, and optionally adding TNF-alpha to the culture at certain stages of differentiation. Thus, cells created in some embodiments are progenitor or precursor cells committed to the T cell lineage (“progenitor T cells”). In some embodiments, the cells are CD7⁺ progenitor T cells. In some embodiments, the cells are CD25⁺ immature T cells, or cells that have undergone CD4 or CD8 lineage commitment. In some embodiments, the cells are CD4⁺CD8⁺ double positive (DP), CD4⁻CD8⁺, or CD4⁺CD8⁻. In some embodiments, the cells are single positive (SP) cells that are CD4⁻CD8⁺ or CD4⁺CD8⁻ and TCR^{hi}. In some embodiments, the cells are TCRαβ⁺ and/or TCRγΔ⁺. In various embodiments, the cells are CD3⁺.

The adoptive transfer of progenitor T cells is a strategy for enhancing T cell reconstitution. Progenitor T cells are developmentally immature and undergo positive and negative selection in the host thymus. Thus, they become restricted to the recipient's major histocompatibility complex (MHC) yielding host tolerant T cells that can bypass the clinical challenges associated with graft-versus-host disease (GVHD). Importantly, engraftment with progenitor T cells restores the thymic architecture and improves subsequent thymic seeding by HSC-derived progenitors. In addition to its intrinsic regenerative medicine properties, progenitor T cells can also be engineered with T cell receptors (TCRs) and chimeric antigen receptors (CARs) (via either gene or mRNA delivery) to confer specificity to tumor-associated antigens.

In various embodiments, the progenitor T cells are further cultured under suitable conditions to generate cells of a desired T cell lineage, including with one or more Notch ligands. For example, the cells can be cultured in the presence of one or more Notch ligands as described for a sufficient time to form cells of the T cell lineage. In some embodiments, stem cells or progenitor T cells are cultured in suspension with soluble Notch ligand or Notch ligand conjugated to particles or other supports, or Notch ligand expressing cells. In some embodiments, the progenitor T cells or stem cells are cultured in suspension or in adherent format in a bioreactor, optionally a closed or a closed, automated bioreactor, with a soluble or conjugated Notch ligand in suspension. One or more cytokines, extracellular matrix component(s), and thymic niche factor(s) that promote commitment and differentiation to the desired T cell lineage may also be added to the culture or reactor. Such cytokines or factors are known in the art. In various embodiments, the HSC population is cultured with the Notch ligand for about 4 to about 21 days, or from about 6 to about 18 days, or from about 7 to about 14 days to generate progenitor T cells. In some embodiments, the stem cell population or derivative thereof is cultured for at least about 21 days or at least about 28 days to generate mature T cell lineages or NK cells.

In various embodiments, the method comprises generating a derivative of the progenitor T cells or generating a T cell lineage from the progenitor T cells. In certain embodiments, the derivative of the progenitor T cell or T cell lineage expresses CD3 and a T cell receptor. In some embodiments, the T cell lineage is CD8⁺ and/or CD4⁺. For example, T cells lineages can include one or more of CD8⁺CD4⁻, CD8⁻CD4⁺, CD8⁺CD4⁺, and CD8⁻ CD4⁻ cells. In some embodiments, the iPSCs, CD34⁺ cells, or derivatives thereof are modified to express a chimeric antigen receptor (CAR) at progenitor-T, T-cell, and/or NK cell level.

In some embodiments, the T cell lineage is a regulatory T cell. T regulatory cells (or T regs) are defined as CD4⁺CD25⁺. Tregs control the immune response to self and foreign antigens and help prevent autoimmune disease. Differentiation of progenitor T cells to Tregs in some embodiments involves ectopic expression of FOXP3 and culturing the progenitor T cells or Treg precursors with one or more growth factors, such as but not limited to IL-2.

In some embodiments, the cell populations or banks of expanded primary cells, or derivatives of iPSCs (e.g., HSC population) are differentiated to B lymphocytes (“B cells”). For example, culturing CD34⁺ or CD34⁺CD43⁺ cells with MS5 stromal cells or S17 stromal cells (e.g., for 15-25 days, or about 21 days) can generate a B-lymphoid identity with expression of CD19, CD45, and CD10. See Carpenter L. et al., Human induced pluripotent stem cells are capable of B-cell lymphopoiesis, Blood 117 (15): 4008-4011. Dubois F. et al., Toward a better definition of hematopoietic progenitors suitable for B cell differentiation, Plos One Dec. 15, 2020. In various embodiments, the B cells produced according to this disclosure express surface IgM (sIgM) and undergo VDJ rearrangement. In various embodiments, B cells produced according to this disclosure will engraft in the spleen and secondary lymphoid tissues of a subject for maturation.

In some embodiments, the cell populations are differentiated to monocytes, macrophages, or neutrophils. For example, erythromyeloid precursors (EMP) (CD43+CD45+) may be generated by culture with IL-6, IL-3, thyroid peroxidase (TPO), SCF, FGF2, and VEGF, followed by differentiation to monocytes. Differentiation to monocytes to employ culture with M-CSF, IL-3, and IL-6. See Cao X et al., Differentiation and Functional Comparison of Monocytes and Macrophages from hiPSCs with Peripheral Blood Derivatives, Stem Cell Reports. 2019 Jun. 11; 12 (6): 1282-1297. Monocytes and macrophage lineages prepared according to this disclosure are CD14+ and will exhibit endocytosis and phagocytic functions. In some embodiments, macrophages are polarized ex vivo to the M1 (pro-inflammatory) or M2 (immunosuppressive) phenotype. In some embodiments, CD45+ hematopoietic cells with phagocytic markers, such as CD33 and CD11b, are generated, and optionally subsequently to cells with neutrophil specific markers, such as CD66b, CD16b, GPI-80, etc., by differentiation of iPSC derived hCD34+ cells. These processes can employ differentiation media containing mixtures of cytokines and growth factors, including but not limited to SCF, IL3, FLT3, IL6, GM-CSF, G-CSF, EPO, TPO, and/or combinations thereof. In some embodiments, neutrophils and their precursors are generated by methods described in: Saeki L., et al., A Feeder-Free and Efficient Production of Functional Neutrophils from Human Embryonic Stem Cells, Stem Cells Vol. 27, Issue 1, 2009, Pages 59-67; Morishima T. et al., Neutrophil differentiation from human-induced pluripotent stem cells. J. Cell. Physiol. 226:1283-1291, 2011; Yokoyama Y. et al., Derivation of functional mature neutrophils from human embryonic stem cells. Blood 2009 Jun. 25; 113 (26): 6584-92; and Sweeney C L et al., Generation of functionally mature neutrophils from induced pluripotent stem cells. Methods Mol Biol 2014; 1124:189-206.

In some embodiments, the HSC population or fraction thereof are differentiated to megakaryocytes or platelets. For example, megakaryocytes (as a renewable source for platelets) can be prepared from the HSCs or fraction thereof by culture with SCF, IL-11, and TPO for several days (e.g., about 5 days). Alternatively, other cytokines and growth factors such as IL-3, IL-6, SDF-1, and FGF-4 can be employed. Megakaryocytes will be CD42b+CD61+. See Liu L., Efficient Generation of Megakaryocytes From Human Induced Pluripotent Stem Cells Using Food and Drug Administration-Approved Pharmacological Reagents, Stem Cells Transl Med. 2015 April; 4 (4): 309-319. Platelets can be further generated from megakaryocytes by culture in serum free media with IL-11. CD41+CD42a+ platelet-like-particles are recovered from the media.

In some embodiments, the derivative of the progenitor T cell is a natural killer (NK) cell. In some embodiments, NK cells are generated from progenitor T cells as described in U.S. Pat. No. 10,266,805, which is hereby incorporated by reference in its entirety. For example, the progenitor T cells can give rise to NK cells when cultured with IL-15. In some embodiments, the NK cell expresses a CAR, based on gene editing of iPSC, embryonic bodies, hCD34+ cells, or NK cells, or via mRNA expression in NK cells.

In some embodiments, the HSC population or fraction thereof is differentiated to red cells or derivatives thereof. Red cells produced according to this disclosure can be administered or used in therapy, for example, for an inherited or acquired red cell disorder, bone marrow failure disorder, high-altitude-related physiological and pathological condition, conditions related to chemicals or radiation exposure, and/or for treatment of subjects undergoing HSC transplant. In further embodiments, the red cells prepared according to this disclosure are provided as a pharmaceutical acceptable composition delivering or encapsulating drugs (including but not limited to enzymes), oxygen carriers, or other suitable materials to treat human disease or physiological or pathological conditions.

In other aspects, the disclosure provides a cell population, or pharmaceutically acceptable composition thereof, according to this disclosure. In some embodiments, the cell population is a lymphocyte population capable of engraftment in a thymus, spleen, or secondary lymphoid organ upon administration to a subject in need. In various embodiments, the composition for cellular therapy is prepared that comprises the desired cell population a pharmaceutically acceptable vehicle.

In some embodiments, the cell composition comprises an iPSC or HSC cell population (or population differentiated therefrom) that is HLA-A^{neg}, HLA-DPB1^{neg}, and HLA-DQB1^{neg}, and is homozygous for, or comprises a single copy of, HLA-B, HLA-C, and HLA-DRB1 as described. Despite such gene deletions and/or gene edits, the iPSCs or HSCs and cells derived (e.g., differentiated therefrom) retain full antigen presenting functionality and ability to differentiate from precursors to hematopoietic lineages (as described herein). Cell compositions of this aspect provide advantages in HLA matching for a recipient, to avoid, for example, GVHD. In various embodiments, the population is homozygous for both HLA-B*08:01 and HLA-C*07:01. In some embodiments, the population is homozygous for HLA-DRB1*03:01. In embodiments, the cells of the composition have an HLA haplotype as described herein.

In various embodiments, the cells of the HSC composition are at least about 50% CD34+, or at least about 60% CD34+, or at least about 75% CD34+, or at least about 80% CD34+, or at least about 85% CD34+, or at least about 90% CD34+, or at least about 95% CD34+. In addition, in embodiments, at least about 50%, or at least about 60%, or at least about 75%, or at least about 80%, or at least about 85%, or at least about 90%, or at least about 95% cells in the composition are one or more of CD90+ and CD45+.

The pharmaceutical composition may comprise at least about 10² cells, or at least about 10³, or at least about 10⁴, or at least about 10⁵, or at least about 10⁶, or at least about 10⁷, or at least about 10⁸ cells, or at least about 10⁹ cells, or at least about 10¹⁰ cells, or at least about 10¹¹ cells, or at least about 10¹² cells, or at least about 10¹³ cells, or at least about 10¹⁴ cells. For example, in some embodiments, the pharmaceutical composition is administered, comprising HSCs of from about 100,000 to about 400,000 cells per kilogram of recipient body weight (e.g., about 200,000 cells/kg). In other embodiments, cells are administered at from about 10⁵ to about 5×10⁵ cells per kilogram (e.g., about 2.5×10⁵ cells/kg), or from about 10⁶ to about 5×10⁶ cells per kilogram (e.g., about 2.5×10⁶ cells/kg), or from about 5×10⁶ to about 10⁷ cells per kilogram (e.g., about 5×10⁶ cells/kg) or from about 10⁷ to about 10⁸ cells per kilogram (e.g., about 5×10⁷ cells/kg) or from about 10⁸ to about 10⁹ cells per kilogram (e.g., about 5×10⁸ cells/kg) or from about 10⁹ to about 10¹⁰ cells per kilogram or from about 10¹⁰ to about 10¹¹ cells or from about 10¹¹ to about 10¹² cells per kilogram or from about 10¹² to about 10¹³ cells per kilogram or from about 10¹³ to about 10¹⁴ cells per kilogram of a recipient's body weight.

The cell composition of this disclosure may further comprise a pharmaceutically acceptable carrier or vehicle suitable for intravenous infusion or other administration route, and the composition may include a suitable cryoprotectant. An exemplary carrier is DMSO (e.g., about 10% DMSO). Cell compositions may be provided in unit vials or bags and stored frozen until use. In certain embodiments, the volume of the composition is from about one fluid ounce to one pint.

In some embodiments, this disclosure provides a CD7+ progenitor T cell, or pharmaceutically acceptable composition thereof, where the CD7+ progenitor T cell produced by a method disclosed herein. In various embodiments, the progenitor T cell is capable of engraftment in a thymus or spleen of a recipient. Progenitor T cells have the potential to decrease the risk of relapse of leukemia or other types of cancer in bone marrow transplant patients and to decrease the number of infections post-transplant that cause significant morbidity and mortality in patients. In another aspect, this disclosure provides a derivative of the progenitor T cell or T cell lineage produced by a method disclosed herein, or a pharmaceutically acceptable composition thereof.

In some embodiments, the cell population is a T cell population (or progenitor T cell population) or NK cell population, which are useful for adoptive cell therapy, for example, for human subjects having a condition selected from lymphopenia, a cancer, an immune deficiency, a viral infection, an autoimmune disease (particularly where the T cell population comprises Tregs), a skeletal dysplasia, a bone marrow failure syndrome, or a genetic disorder that impairs T cell development or function. Exemplary genetic disorders can impact the immune system, manifesting as an immunocompromised state, or autoimmune or pro-inflammatory state. In some embodiments, the subject has cancer, which is optionally a hematological malignancy or a solid tumor. In some embodiments, the T cell is a CAR-T cell.

In some embodiments, the cell population is a B lymphocyte population, and is capable of engraftment in a spleen or secondary lymphoid tissue of a subject. B-cell populations according to this disclosure have the potential to partially reconstitute humoral immunity in an immune compromised patient, for example, providing protection from or treatment for infectious diseases, including viral, bacterial, fungal, or parasite infection. In various embodiments, the B cells according to this disclosure are capable of differentiation to plasma cells for production of antigen-specific antibodies in vivo. In other embodiments, B cells produced according to this disclosure can be employed for cancer immunotherapy. In some embodiments, chimeric antigen B cells (CAR B cells) are prepared by gene modifications at iPSC, embryonic bodies, hCD34+ cells, hematopoietic progenitor cell, or B cell level. CAR B cells express a surface BCR and/or secrete a recombinant monoclonal antibody that recognizes a target antigen, such as a cancer antigen or an infectious disease antigen. In still other embodiments, B cells produced according to this disclosure are used for ex vivo production of antibodies (e.g., vaccine antibodies for providing protection from an infectious agent).

In some embodiments, the cell population is a monocyte or macrophage cell population, and the cell population is capable of engraftment and maturation in various tissues of a subject, including tumors. In various embodiments, the monocyte or macrophage cell population is able to form tissue resident macrophages in a subject. In various embodiments, the macrophages are predominately of the M1 (pro-inflammatory) or M2 (immunosuppressive) phenotype. In various embodiments, the subject in need to treatment has a cancer of any of various tissues or organs, liver or kidney inflammatory disease, or bacterial infection (e.g., sepsis or infection or colonization of an indwelling medical device).

In some embodiments, the cell population is a megakaryocyte population, or is platelets developed therefrom. These cells or platelets are useful for treating inherited platelet defects, impacting for example, coagulation pathways.

In some embodiments, the cell population is a red cell population.

In still other embodiments, the iPSCs are differentiated to non-hematopoietic stem cells or precursor cells, or cells or tissues differentiated therefrom. Such cells include mesenchymal stem cells, neural stem cells, epithelial stem cells, neuronal cells (or precursors thereof) (including cortical, dopaminergic, and motor neurons, or precursors thereof), astrocytes (or precursors thereof), oligodendrocytes (or precursors thereof), cardiomyocytes (or precursors thereof), skeletal muscle cells (or precursors thereof), hepatocytes (or precursors thereof), pancreatic β cells (or precursors thereof), and lung epithelial cells (or precursors thereof).

The cell composition of this disclosure may further comprise a pharmaceutically acceptable excipient or a carrier. Such excipients or carrier solutions also can contain buffers, diluents, and other suitable additives. Further, the composition may comprise a vehicle suitable for intravenous infusion or other administration route, and the composition may include a suitable cryoprotectant. An exemplary carrier is DMSO (e.g., about 10% DMSO) Other carriers may include dimethoxy ethane (DME), N,N-dimethylformamide (DMF), or dimethylacetamide, including mixtures or combinations thereof. Cell compositions may be provided in implantable devices (e.g., scaffolds) or in bags or in vials, tubes or a container in an appropriate volume and stored frozen until use.

The derivatives of cell line(s) or banks of expanded primary cells (e.g., HSCs and progenies thereof) can be formulated, dosed, and administered in a fashion consistent with good medical practice. Factors for consideration in this context include the particular disease or disorder being treated, the particular mammal being treated (e.g., human), the clinical condition of the individual patient, the cause of the disease or disorder, the site of delivery of the agent, the method of administration, the scheduling of administration, and other factors known to medical practitioners. The therapeutically effective amount of the cells (HSCs or progenitors or progenies derived from the cell line(s) or banks of expanded primary cells or derivatives of iPSC cells or banks thereof) to be administered will be governed by such considerations.

One may administer other compounds, such as cytotoxic agents, immunosuppressive agents and/or cytokines or growth factors (e.g., stem cell factor, thrombopoietin, transforming growth factor (TGF)-α or β, fibroblast growth factors (FGF), angiopoietin (Ang) family of growth factors, insulin-like growth factors, granulocyte-macrophage colony-stimulating factor, TNF-α or β, VEGF, interleukins (e.g., IL-2, 6, 7, 8 10, 12, 15 etc.,) and interferons (e.g., INF-alpha or gamma)) with the cells (e.g., HLA modified iPSC-derived HSCs or progenitors or progenies thereof) herein. The combined administration includes co-administration, using separate formulations or a single pharmaceutical formulation, and consecutive administration in either order, wherein preferably there is a time period while both (and all) active agents simultaneously exert their biological activities.

The present pharmaceutical compositions may be administered in any dose appropriate to achieve a desired outcome. In some embodiments, the desired outcome is a reduction in the intensity, severity, frequency, and/or delay of onset of one or more symptoms of infection. In some embodiments, the desired outcome is the inhibition or prevention of infection. The dose required will vary from subject to subject depending on the species, age, weight, and general condition of the subject, the severity of the infection being prevented or treated, the particular composition being used, and its mode of administration.

The formulations to be used for in vivo administration must be sterile. This is readily accomplished by filtration through sterile filtration membranes and other techniques known to one of skill in the art.

As used herein, unless the context requires otherwise, the term “about” means ±10% of the associated numerical value.

Certain aspects and embodiments of this disclosure are further described with reference to the following examples.

### EXAMPLES

### Example 1: IPSC-Derived HSCs Generated with Piezo1 Activation Undergo T Cell Differentiation Similar to Bone Marrow-Derived HSCs

### Methods

iPSCs were developed from hCD34+ cells by episomal reprogramming as known in the art and essentially as described in Yu, et al. Induced pluripotent stem cell lines derived from human somatic cells, Science 318, 1917-1920, (2007); and J. Yu, et al. Human induced pluripotent stem cells free of vector and transgene sequences. Science 324, 797-801, (2009). Embryoid Bodies and hemogenic endothelium differentiation was performed essentially as described in: R. Sugimura, et al., Haematopoietic stem and progenitor cells from human pluripotent stem cells. Nature 545, 432-438, (2017); C. M. Sturgeon, et al, Wnt signaling controls the specification of definitive and primitive hematopoiesis from human pluripotent stem cells. Nat Biotechnol 32, 554-561, (2014); J. Yu, et al. Induced pluripotent stem cell lines derived from human somatic cells. Science 318, 1917-1920, (2007); and J. Yu, et al. Human induced pluripotent stem cells free of vector and transgene sequences. Science 324, 797-801, (2009).

Briefly, hiPSC were dissociated and resuspended in media supplemented with L-glutamine, penicillin/streptomycin, acid, ascorbic human holo-Transferrin, monothioglycerol, BMP4, and Y-27632. Next, cells were seeded in 10 cm dishes (EZSPHERE or low attachment plate) for the EB formation. On Day 1, bFGF and BMP4 were added to the medium. On Day 2, the media was replaced with a media containing SB431542, CHIR99021, bFGF, and BMP4. On Day 4, the cell media was replaced with a media supplemented with VEGF and bFGF. On day 6, the cell media was replaced with a media supplemented with bFGF, VEGF, IL-6, IGF-1, IL-11, SCF, and EPO. Cells were maintained in a 5% CO₂, 5% O₂, and 95% humidity incubator. To harvest the CD34+ cells, the EBs were dissociated on day 8, cells were filtered through a 70 μm strainer, and CD34+ cells were isolated by CD34 magnetic bead staining.

EB-derived CD34+ cells were suspended in medium containing Y-27632, TPO, IL-3, SCF, IL-6, IL-11, IGF-1, VEGF, bFGF, BMP4, and FLT3. After the cells had adhered to the bottom of the wells for approximately 4-18 hours (by visual inspection), Yoda1 was added to the cultures for some experiments. After 4-7 days, the cells were collected for analysis of endothelial-to-hematopoietic transition (EHT).

For lineage experiments, iPSCs were differentiated to embryoid bodies for 8 days, as described. At day 8, CD34+ cells from iPSC-derived embryoid bodies were harvested and cultured for additional 5 to 7 days to induce endothelial-to-hematopoietic (EHT) transition (with and without Yoda1). Then, CD34+ cells were harvested from the EHT culture between day 5 to day 7 for further hematopoietic lineage differentiation.

CD34+ cells, harvested from the EHT culture between day 5-7 (or total of day 13-21 differentiation from iPSCs), were seeded in 48-well plates pre-coated with rhDL4 and RetroNectin. T lineage differentiation was induced in media containing aMEM, FBS, ITS-G, 2BME, ascorbic acid-2-phosphate, Glutamax, rhSCF, rhTPO, rhIL7, FLT3L, rhSDF-1a, and SB203580.

Between day 2 to day 6, 80% of the media was changed every other day. At D7, cells were transferred into new coated plates and analyzed for the presence of pro-T cells (CD34+CD7+CD5+/−).

Between day 8 to day 13, 80% of the media was changed every other day. At D14, 100,000 cells/wells were transferred to a new coated plate and the cells analyzed for the presence of pre-T cells (CD34-CD7+CD5+/−).

Between day 15 to day 20, 80% of the media was changed every other day. Cells were harvested at D21, and the cells were analyzed for CD3, CD4, CD8, CD5, CD7, TCRab expression, as surrogates for T cells, via FACS, and/or activated using CD3/CD28 beads to evaluate their functional properties.

After 21 days of differentiation, cells were collected and re-seeded at approximately 80,000 cells into new 96-well culture plates in RPMI 1640 (no L-glutamine; no phenol red) plus FBS, L-glutamine, IL-2, and then activated with 1:1 CD3/CD28 beads. After 72 hours of activation with CD3/CD28 beads, cells were analyzed for CD3, CD69, CD25 expression by FACS and IFN-γ expression using RT-qPCR. The supernatant was analyzed by ELISA.

### Results

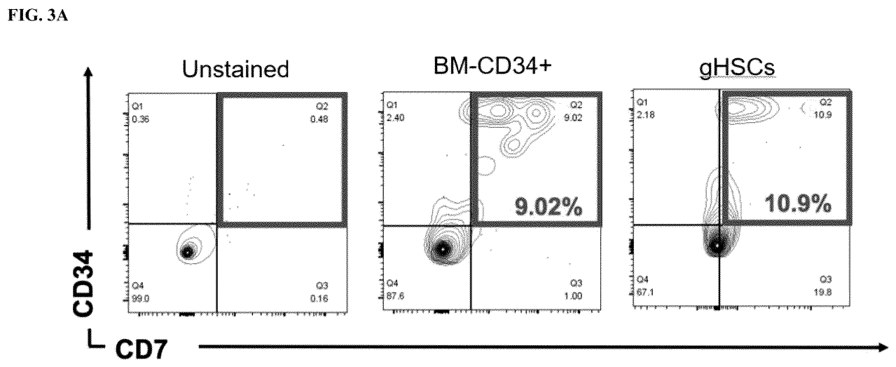

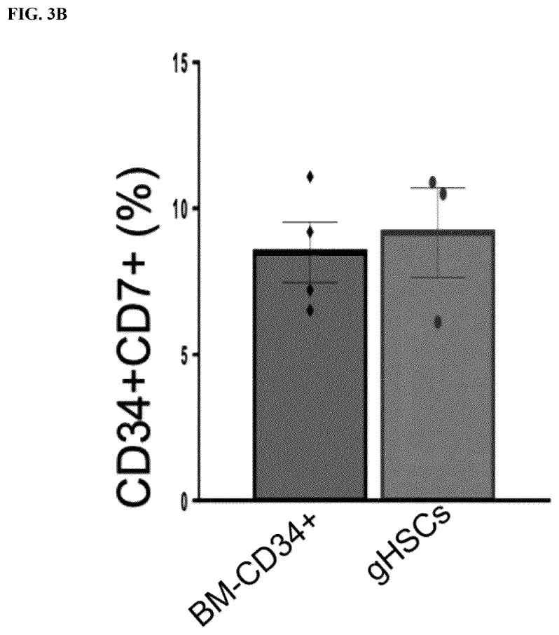

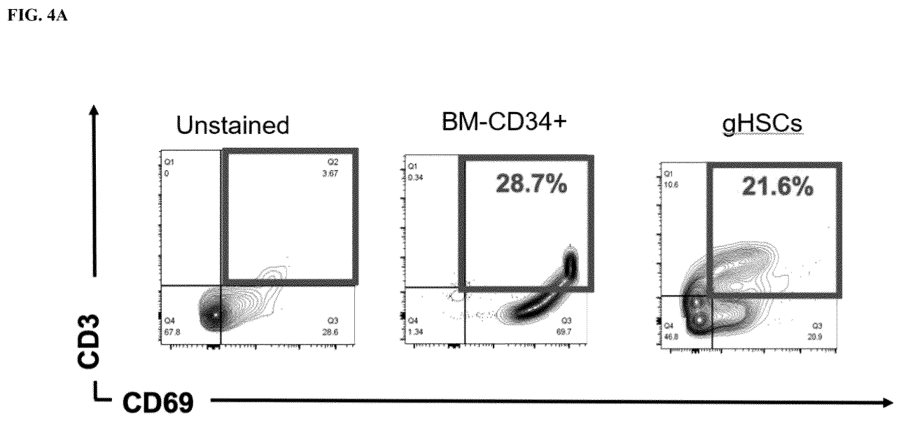

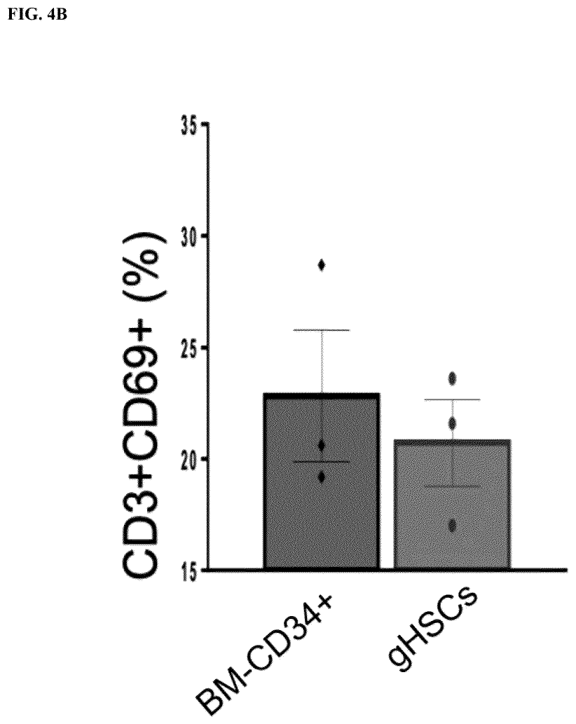

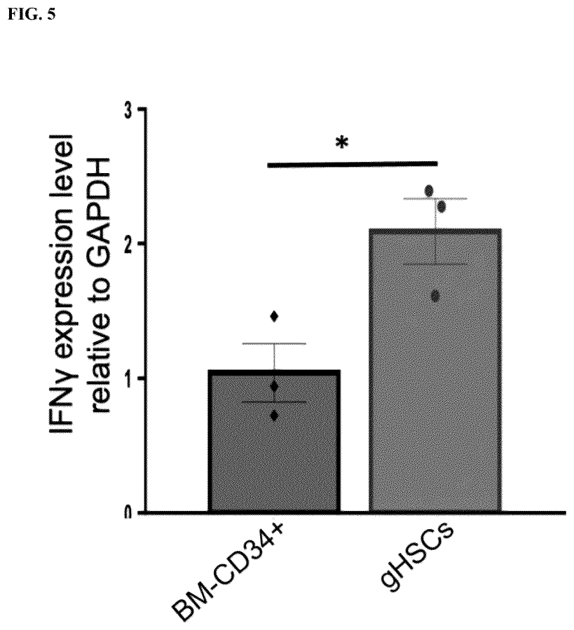

FIG. 3A and FIG. 3B show that iPSC-derived HSCs that are derived with EHT of CD34+ cells from differentiated iPSCs (e.g., in this case including Piezo1 activation) undergo pro-T cell differentiation similar to bone marrow (BM)-HSCs. Further, FIG. 4A and FIG. 4B show that iPSC-derived HSCs generated with EHT of CD34+ cells from differentiated iPSCs (in this case involving Piezo1 activation) undergo T cell differentiation and can be activated with CD3/CD28 beads similar to BM-HSCs. FIG. 5 shows that iPSC-derived HSCs (in this case generated with Piezo1 activation) can differentiate to functional T cells, as demonstrated by INFγ expression upon stimulation with CD3/CD28 beads. Together, these results demonstrate that iPSC-derived HSCs (i.e., derived with EHT of CD34+ cells from differentiated iPSCs) enhances HSC ability to further differentiate to hematopoietic lineages ex vivo, such as progenitor T cells and functional T cells.

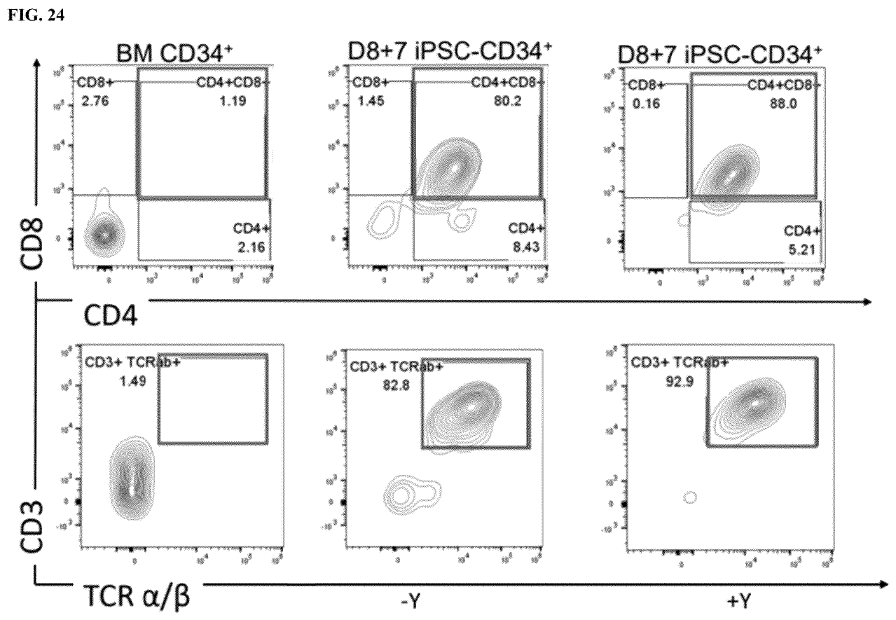

FIG. 24 shows that HSCs generated according to this disclosure (labeled as D8+7 iPSC-CD34+) successfully differentiate into CD4+CD8+ (“double positive”) T cells as well as TCR α/β T cells. The methods of the present disclosure substantially outperform bone marrow CD34+ cells for T cell maturation. FIG. 24 shows results with (“+Y”) and without (“−Y”) Yoda1 during HSC formation.

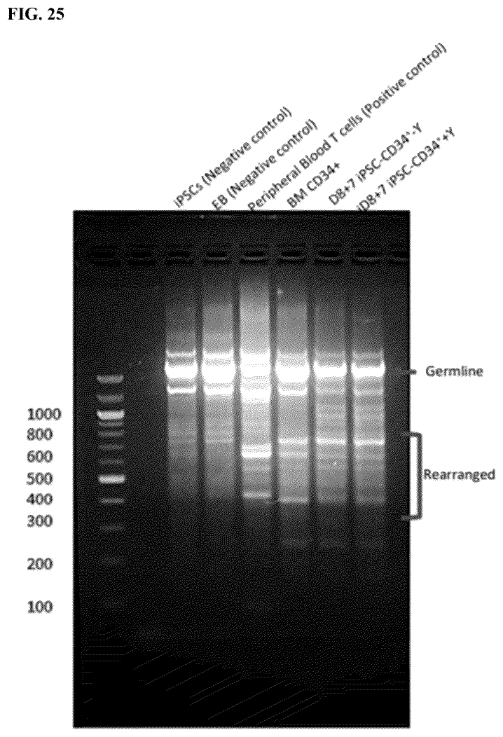

FIG. 25 shows that HSCs generated according to this disclosure (D8+7 iPSC-CD34+ cells (+ or − Yoda1) successfully rearrange TCR, and outperform bone marrow CD34⁺ cells. Shown are iPSC and EB negative controls, Peripheral Blood T cells as positive control, T cells generated from BM CD34⁺ cells, and T cells generated according to this instant disclosure with and without Yoda1.

### Example 2: CCR5 Deletion

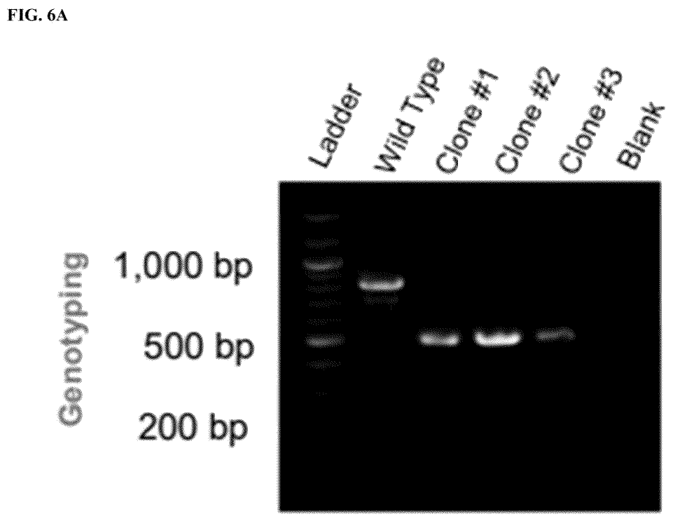

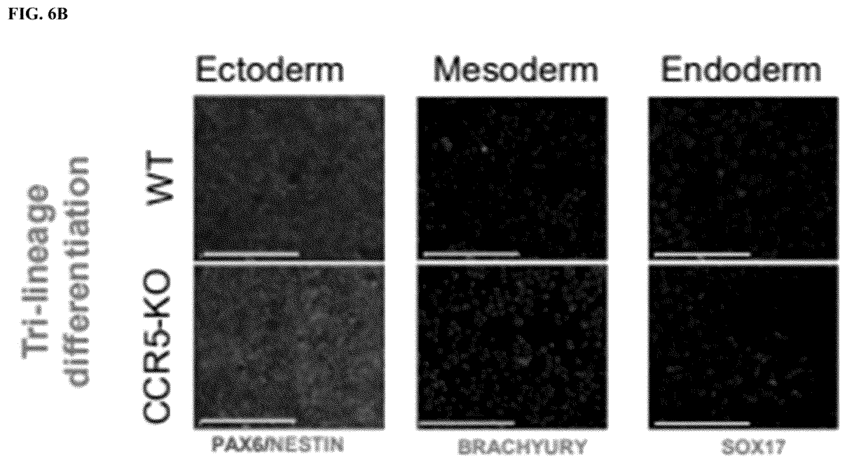

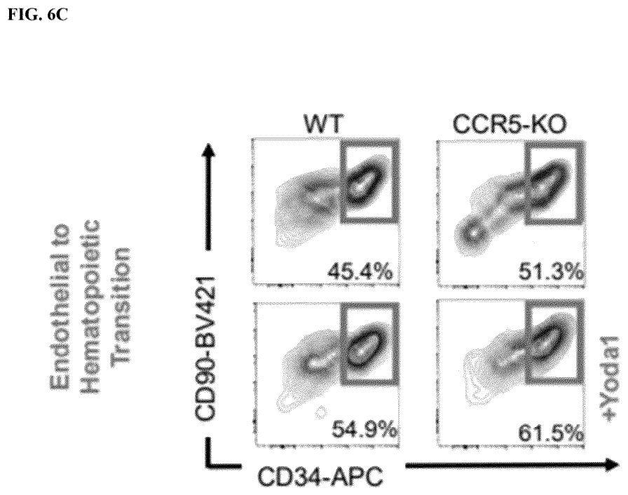

FIG. 6A shows generation of three CCR5-knockout (KO) iPSC clones. As shown in FIG. 6B, the CCR5-KO does not affect the iPSC pluripotency. Further, as shown in FIG. 6C, CCR5-KO does not affect the ability of cells to undergo the endothelial-to hematopoietic transition.

### Example 3: CD33 Deletion

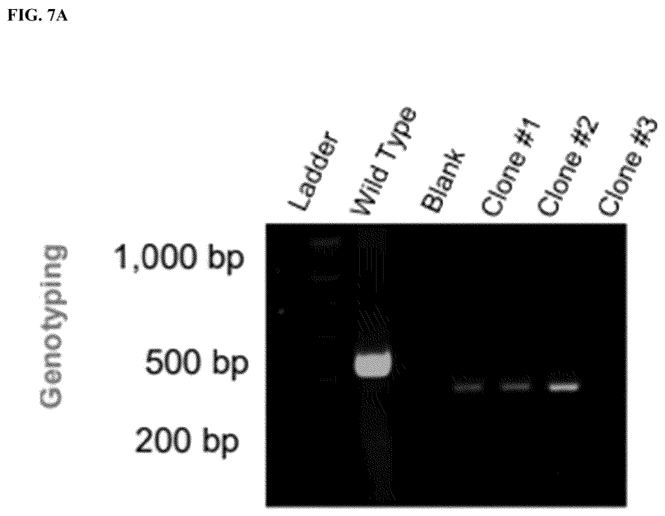

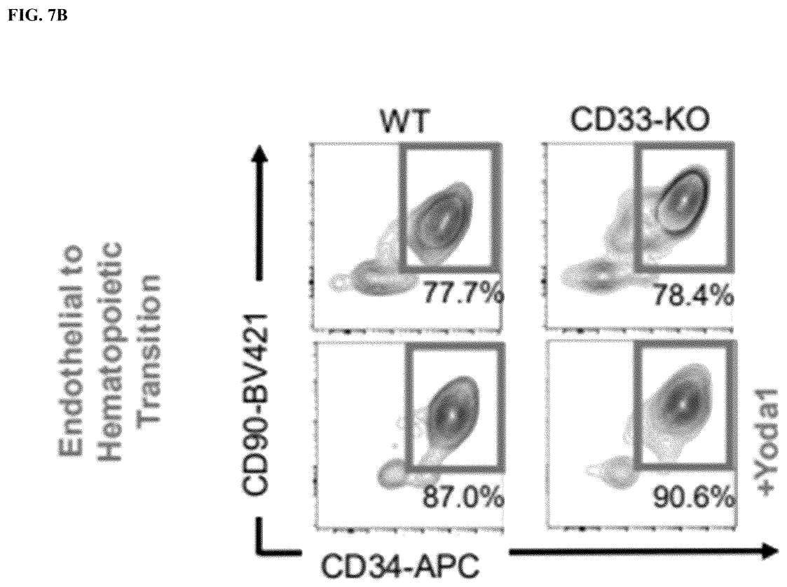

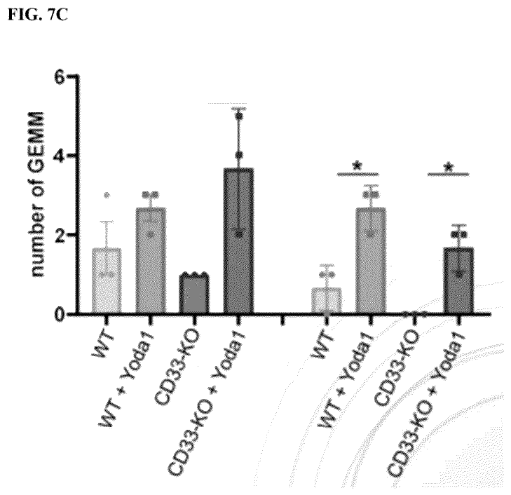

FIG. 7A shows generation of three CD33-KO iPSC clones. As shown in FIG. 7B, CD33-KO does not affect the ability of cells to undergo the endothelial-to hematopoietic transition. Further, CD33-KO does not affect the ability of cells to generate self-renewing HSCs (FIG. 7C).

### Example 4: HLA Editing

According to the disclosure, cells can be HLA-modified by CRISPR-Cas9 using one or more of the following gRNA comprising s spacer sequence shown in Table 2. gRNA were designed using the following parameters: target sequences as shown below were targeted to Exon 2 (to affect peptide groove) of each variant; PAM motifs with 3′NGG, and target length of 20 nucleotides. Table 2 provides spacer sequences of gRNAs used in the experiments. The gRNA sequences can be used to knock out expression of indicated HLA genes.

TABLE 2
Exemplary gRNA sequences

| gRNA ID | Spacer sequence |
|---|---|
| HLA-A | CGTAGCCCACGGCGATGAAG (SEQ ID NO: 1) |
| | ATTTCTTCACATCCGTGTCC (SEQ ID NO: 2) |
| | GTCTCCTGGTCCCAATACTC (SEQ ID NO: 3) |
| | GTAGCCCACGGCGATGAAGC (SEQ ID NO: 4) |
| | TAGCCCACGGCGATGAAGCG (SEQ ID NO: 5) |
| | GAGGGTTCGGGGCGCCATGA (SEQ ID NO: 6) |
| | CCGGAACACACGGAATGTGA (SEQ ID NO: 7) |
| | ACAGCGACGCCGCGAGCCAG (SEQ ID NO: 8) |
| | CCACTCACAGATTGACCGAG (SEQ ID NO: 9) |
| | CGACGCCGCGAGCCAGAGGA (SEQ ID NO: 10) |
| | CAGATTGACCGAGTGGACCT (SEQ ID NO: 11) |
| | CAGACTGACCGAGTGGACCT (SEQ ID NO: 12) |
| | ACAGACTGACCGAGTGGACC (SEQ ID NO: 13) |
| | GCCGGGCCGGGACACGGATG (SEQ ID NO: 14) |
| | CCAGGAGACACGGAATGTGA (SEQ ID NO: 15) |
| | CCAGTCACAGACTGACCGAG (SEQ ID NO: 16) |
| | TCGACAGCGACGCCGCGAGC (SEQ ID NO: 17) |
| HLA-DPB1 | GAGATACATCTACAACCGGG (SEQ ID NO: 18) |
| | TGGAGAGATACATCTACAAC (SEQ ID NO: 19) |
| | GGTTGTAGATGTATCTCTCC (SEQ ID NO: 20) |
| | GGAGAGATACATCTACAACC (SEQ ID NO: 21) |
| | AGGAATGCTACGCGTTTAAT (SEQ ID NO: 22) |
| HLA-DQB1 | CAGATACATCTATAACCGAG (SEQ ID NO: 23) |
| | CGAGTACTGGAACAGCCAGA (SEQ ID NO: 24) |
| | CCCGTTGGTGAAGTAGCACA (SEQ ID NO: 25) |
| | GTGCTACTTCACCAACGGGA (SEQ ID NO: 26) |
| | AGGTCGTGCGGAGCTCCAAC (SEQ ID NO: 27) |
| | AGGTGACAGTTGCTACCCGA (SEQ ID NO: 28) |
| | TCCCGTTGGTGAAGTAGCAC (SEQ ID NO: 29) |
| | AACTACGAGGTGGCGTACCG (SEQ ID NO: 30) |
| | CTGTTCCAGTACTCGGCAAC (SEQ ID NO: 31) |
| | ACTACGAGGTGGCGTACCGC (SEQ ID NO: 32) |
| | CGAAGCGCACGTACTCCTCT (SEQ ID NO: 33) |

Candidate gRNAs were evaluated for potential off-target editing, with gRNAs shown in Table 3 resulting in select clones 1-15 summarized in Table 4.

TABLE 3
gRNA target sequences for disrupting HLA-A, HLA-DPB1, HLA-DQB1, and
HLA-DQB1.

| Target gene | gRNA name | Target sequence | PAM |
|---|---|---|---|
| HLA-A | HLA- | GAGGGTTCGGGGCGCCATGA | CGG |
| | A_Ex1_GBS_gRNA1 | (SEQ ID NO: 6) | |
| HLA-DPB1 | DPB10101-D | GGAGAGATACATCTACAACC | GGG |
| | | (SEQ ID NO: 21) | |
| HLA-DQB1 | DQB10301-D | GTGCTACTTCACCAACGGGA | CGG |
| | | (SEQ ID NO: 26) | |
| HLA-DQB1 | DQB10301-E | AGGTCGTGCGGAGCTCCAAC | TGG |
| | | (SEQ ID NO: 27) | |

The HLA genes are located on the short arm of chromosome 6 (e.g., as shown in FIG. 8), and because the HLA system contains closely related genes, specific gRNA design is challenging. Sequencing of the triple knockout (HLA edited) clones was performed to check for unwanted editing and to ensure that no major editing events, e.g., deletion(s), occurred within other regions of chromosome 6. Sequencing methods and other analyses were performed to evaluate the degree of gRNA off-target activity and to select gRNAs that represent a low risk of affecting non-target HLA genes.

Sequencing was performed by using in situ break labelling in fixed and permeabilized cells by ligating a full-length P5 sequencing adapter to end-prepared DSBs. Genomic DNA was extracted, fragmented, end-prepared, and ligated using a chemically modified half-functional P7 adapter. The resulting DNA libraries contained a mixture of functional DSB-labelled fragments (P5:P7) and non-functional genomic DNA fragments (P7:P7). Subsequent DNA sequencing of the DNA libraries enriched for DNA-labelled fragments, eliminating all extraneous non-functional DNA. As the library preparation is PCR-free, each sequencing read obtained was equivalent to a single labelled DSB-end from a cell. This generated a DNA break readout, enabling the direct detection and quantification of genomic DSBs by sequencing without the need for error-correction and enabled mapping a clear list of off-target mutations.

Table 4 below summarizes the results of the editing strategy in representative HLA edited clones relative to non-edited (gHSCs) cells using the gRNA sequences from Table 3.

TABLE 4
Clonal HSC HLA knockouts.

| Sample ID | Locus | Allele 1 | Allele 2 | Comments |
|---|---|---|---|---|
| gHSCs | A | A*01:01:01 | A*01:01:01 | Not affected |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | DPB1*01:01:01 | DPB1*04:01:01 | Not affected |
| | DQA1 | DQA1*05:01:01 | DQA1*05:01:01 | Not affected |
| | DQB1 | DQB1*02:01:01 | DQB1*02:01:01 | Not affected |
| | DRB1 | DRB1*03:01:01 | DRB1*03:01:01 | Not affected |
| | DRB345 | DRB3*01:01:02 | DRB3*01:01:02 | Not affected |
| Clone #1 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #2 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #3 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Insertion in Exon 2 |
| | DQA1 | DQA1*05:01:01 | DQA1*05:01:01 | Not affected |
| | DQB1 | xxx | xxx | insertion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #4 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Insertion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #5 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #6 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #7 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #8 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Insertion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #9 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #10 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #11 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Insertion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #12 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Indel in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #13 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #14 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Insertion in Exon 2 |
| | DQA1 | DQA1*05:01:01 | DQA1*05:01:01 | Not affected |
| | DQB1 | xxx | xxx | Insertion in Exon 2 |
| | DRB1 | DRB1*03:01:01 | DRB1*03:01:01 | Not affected |
| | DRB345 | DRB3*01:01:02 | DRB3*01:01:02 | Not affected |
| Clone #15 | A | xxx | xxx | Deletion in Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in Exon 2 |
| | DQA1 | DQA1*05:01:01 | DQA1*05:01:01 | Not affected |
| | DQB1 | xxx | xxx | Deletion in Exon 2 |
| | DRB1 | DRB1*03:01:01 | DRB1*03:01:01 | Not affected |
| | DRB345 | DRB3*01:01:02 | DRB3*01:01:02 | Not affected |

The results show that the editing strategy was successful in selectively targeting the HLA-A, DPB1, and DQB1 genes without affecting the other HLA genes or introducing major deletion elsewhere.

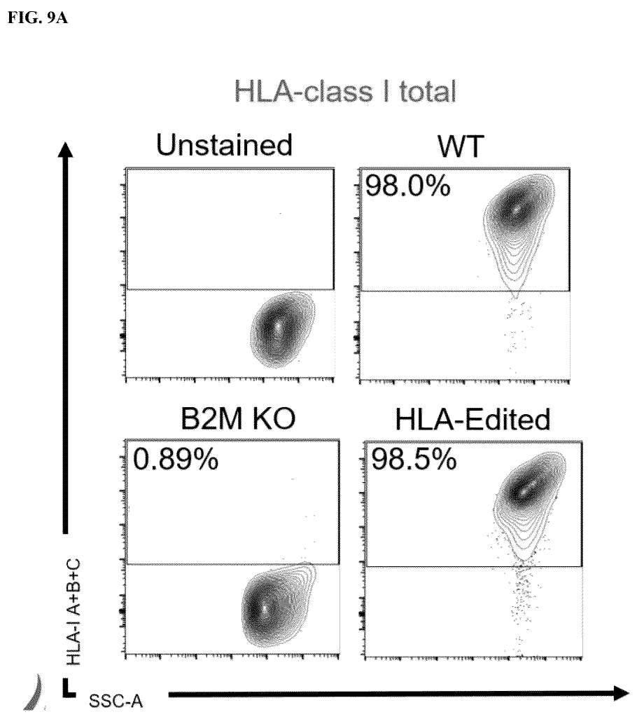

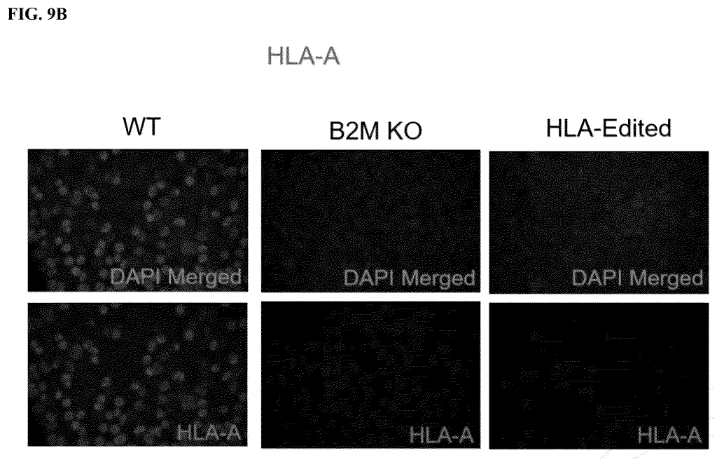

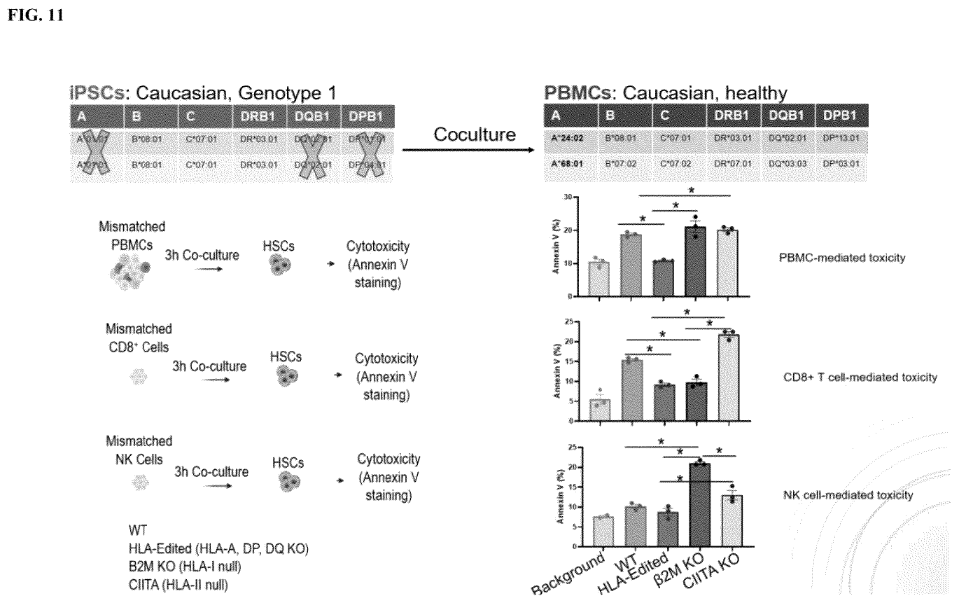

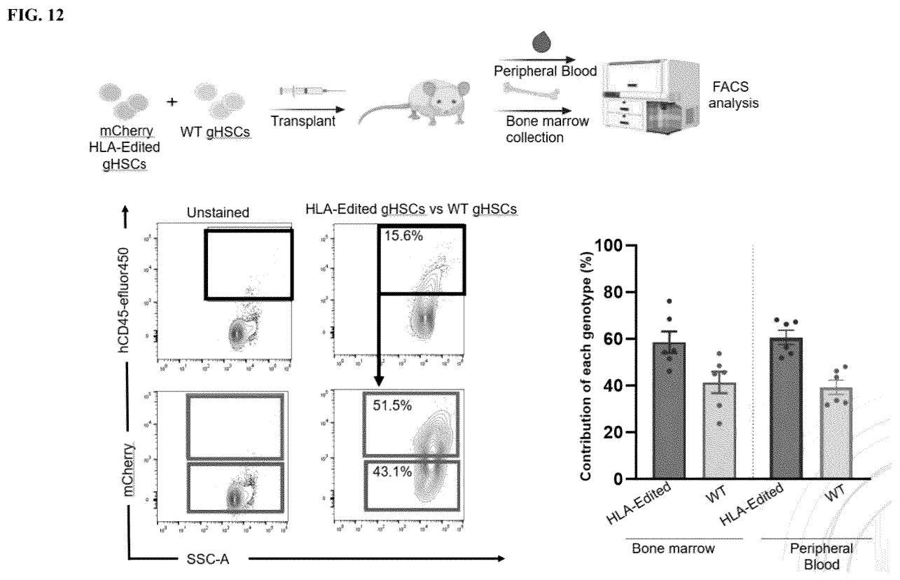

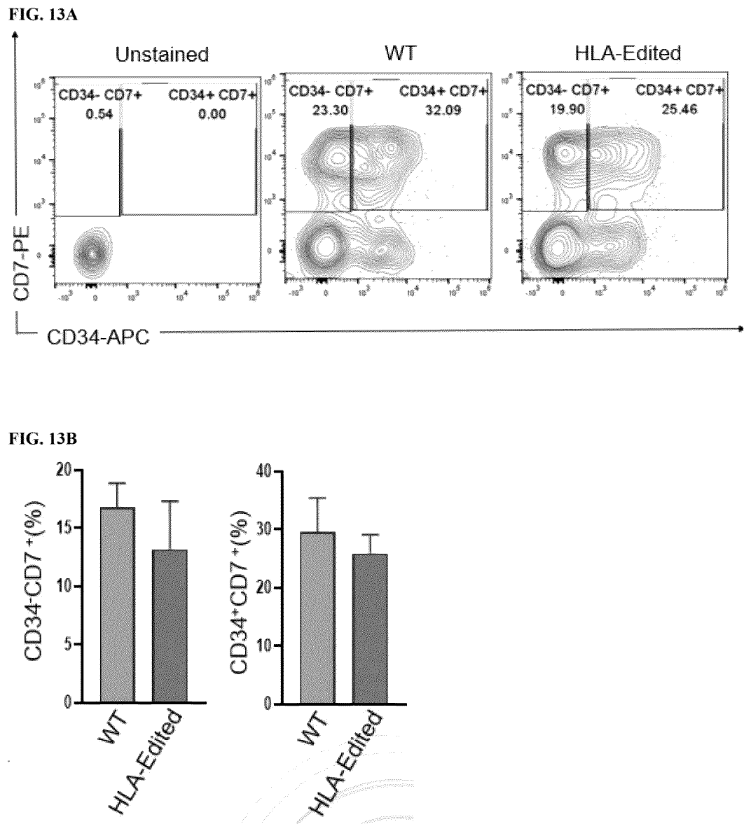

These results were confirmed by a phenotypic analysis of the HLA edited clones by FACS and immunofluorescence. As shown in FIGS. 12A and B, HLA edited cells tested positive for overall expression of HLA class-I molecules, comparable to the overall expression of HLA class-I molecules of wild-type cells. Specific expression of HLA-A via immunofluorescence confirmed that HLA-A was not expressed in the HLA edited cells, corroborating the finding that the gene editing strategy was successful in deleting only the HLA-A gene. Specifically, FIG. 9A, shows that the HLA edited cells were all positive for class-I like HLA to the same extent as the wild type (gHSC) cells. This result indicates that despite the deletion of HLA-A, other class-I molecules like HLA-B and C were expressed and not affected by the gene editing strategy.

To confirm that the HLA-A gene was deleted, specific expression of the HLA-A was analyzed with immunofluorescence. As can be seen in FIG. 9B, HLA-A was not expressed in the HLA edited clone indicating that the gene editing strategy was efficient in specifically deleting the HLA-A gene only. Such preservation of overall class-I expression with deletion of HLA-A will facilitate patient matching while avoiding NK-cell mediated rejection.

### Example 5: Evaluating Pluripotency and Immunecompatability of HLA Edited HSCs

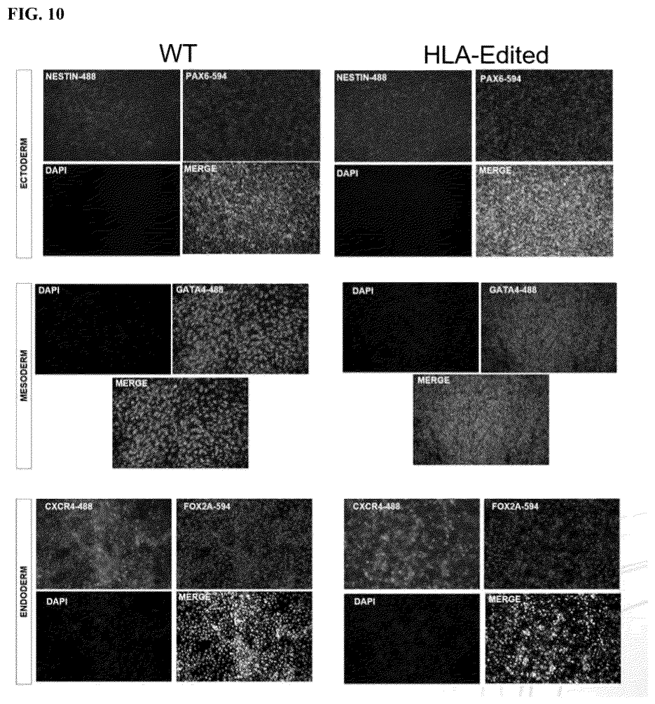

The ability of HLA edited cells to preserve pluripotency was evaluated. As shown in FIG. 10, immunofluorescence evaluation of the HLA edited iPSC clones indicated that they maintained trilineage differentiation, with ectoderm differentiation indicated by NESTIN-488 and PAX6-594 staining, mesoderm differentiation indicated by GATA-488 staining, and endoderm differentiation indicated by CXCR4-488 and FOX2A-594 staining.

HLA class I molecules are expressed on the surface of all nucleated cells and if the HLA class I molecules are mismatched between donor and recipient, then the cells could be recognized and killed by CD8+ T cells. Additionally, HLA mismatching could lead to cytokine release syndrome (CRS) and graft-versus-host disease (GVHD). Conversely, the complete deletion of HLA-I molecules, via B2M KO, would make the cell a target of NK cell-mediated cytotoxicity. The preservation of overall class-I expression with deletion of HLA-A can facilitate patient matching while preventing the NK-cell mediated rejection. Thus, the immunocompatibility of the HLA edited HSCs was tested by co-culture with peripheral blood mononuclear cells (PBMCs) to evaluate if the immune cells would reject a graft of the HLA edited and gHSCs.

Non-edited (gHSCs) and HLA edited HSCs were co-cultured with PBMCs matching the HLA-B and HLA-C markers, but with mismatched HLA-A. B2M KO HSCs lacking expression of HLA class-I molecules and CIITA KO HSCs lacking expression of class-II molecules were used as controls to compare the degree of PBMC-mediated cytotoxicity for HLA-null and mismatched HLA, respectively. FIG. 11 shows the results of the PBMC-mediated cytotoxicity assay in the co-cultures as measured by an annexin V staining. The results show that deletion of HLA-A in the HLA edited HSCs protects the cells from PBMC-mediated cytotoxicity, while WT, B2M KO, and CIITA KO were susceptible to PBMC-mediated cytotoxicity. Co-cultured HSCs with sorted CD8+ T cells from the same PBMC donor protected HLA edited and B2M KO HSCs from CD8+ T cell cytotoxicity. Conversely, co-cultured HSCs with sorted NK cells only protected the WT (gHSCs) and HLA edited cells from the NK cell-mediated cytotoxicity.

In summary, the immune capability results show that the CD8+ T cells present in the PBMC samples were responsible for killing the cells with mismatched HLA molecules (non-edited) and CIITA KO, while the NK cells present in the PBMCs were responsible for killing the HLA-null cells (B2M KO). However, HLA edited HSCs are protected from CD8+ T cell-mediated cytotoxicity (because the mismatched HLA-A had been knocked out), and protected from NK cell-mediated cytotoxicity (because HLA class I molecule expression was largely preserved).

### Example 6: Evaluating the In Vivo Engraftment Potential of HLA Edited HSCs

To evaluate the engrafting potential of HLA edited HSCs, the cells' ability to engraft in vivo was evaluated by a competitive transplant against WT HSCs. Equal proportions of mCherry HLA edited HSCs and unedited HSCs were admixed and transplanted into mice, from where bone marrow (BM) and peripheral blood samples were recovered and evaluated by FACS to compare the relative amounts of each cell type present in the samples. As shown in FIG. 12, both the HLA edited HSCs and the WT HSCs contributed to approximately equal engraftment in the BM and peripheral blood samples. These results confirm that HLA edited HSCs (prepared according to this disclosure) are comparable to WT HSCs in their engraftment and reconstitution potential. Hence, it is expected that properties of the WT HSCs are consistent with that of the HLA edited HSCs of the present disclosure.

### Example 7: Differentiation of HLA Edited HSCs to Hematopoietic Lineages

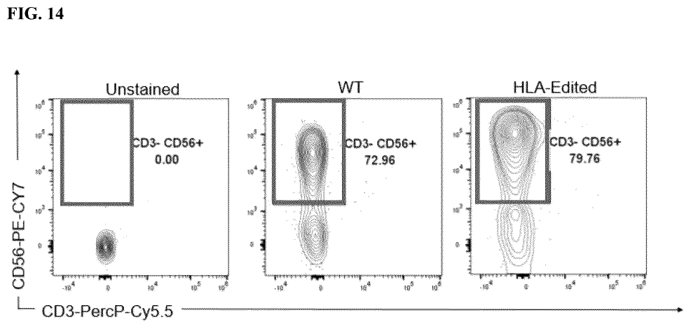

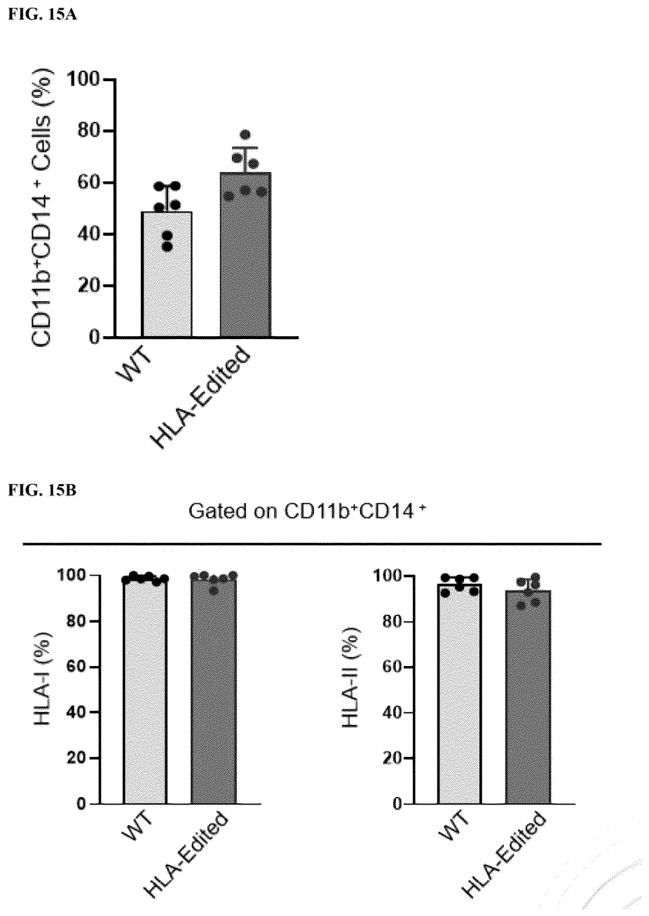

Experiments were carried out to determine if HLA deletion impacts the HSCs ability to differentiate into different types of immune cells. Using the process essentially as described in Example 2, HLA edited HSCs were differentiated to pro-T cells. It was found that the HLA-edited HSCs were able to differentiate into Pro-T Cells, which was comparable to WT (non-HLA-edited) HSCs as measured by their CD34+-CD7+ expressions (FIS. 13A and B). Further, upon differentiation to NK cells, it was shown that HLA-edited HSCs were able to differentiate into NK cells, comparable to WT HSCs as measured by their CD3-CD56+ expressions (FIG. 14). Likewise, it was determined that the HLA-edited HSCs were able to differentiate into monocyte/macrophage lineage comparable to WT HSCs as measured by their CD11b+-CD14+ expressions (FIG. 15A). Further, the CD11b+-CD14+ gated population showed equivalent HLA-I and HLA-II expression (FIG. 15B) indicating that HLA-edited HSCs preserve the overall expression of both class I and class II molecules.

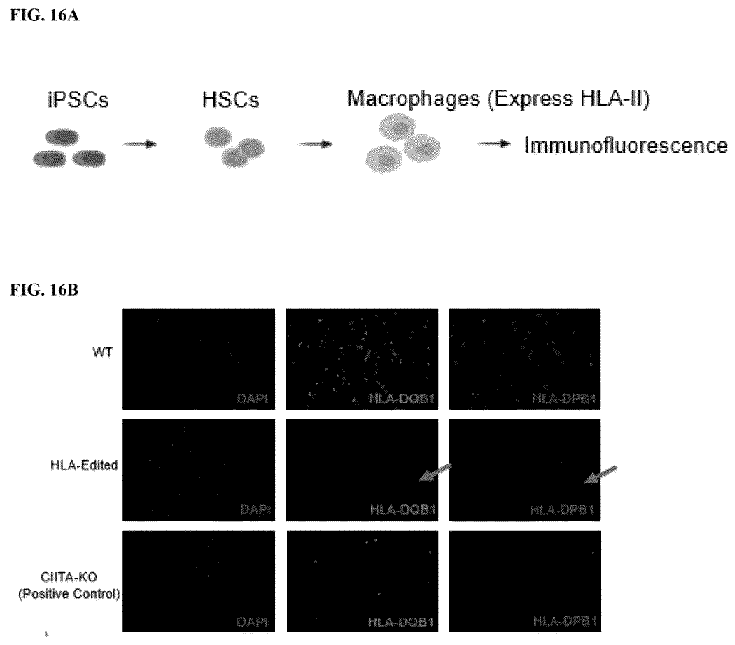

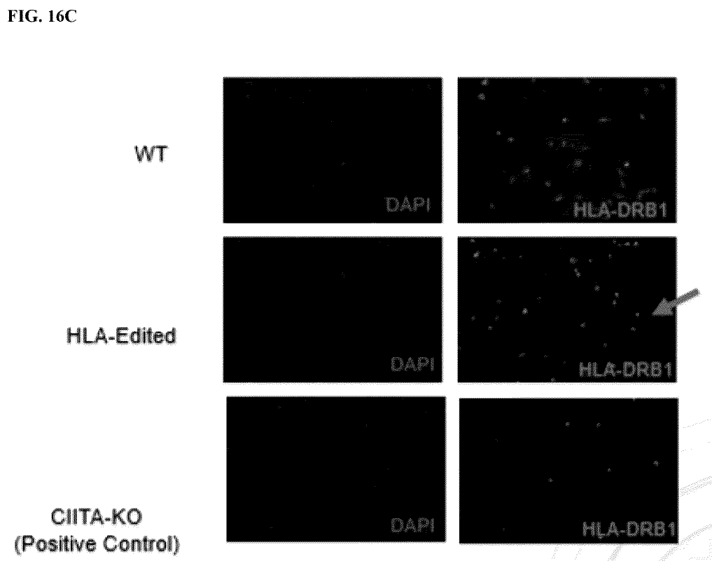

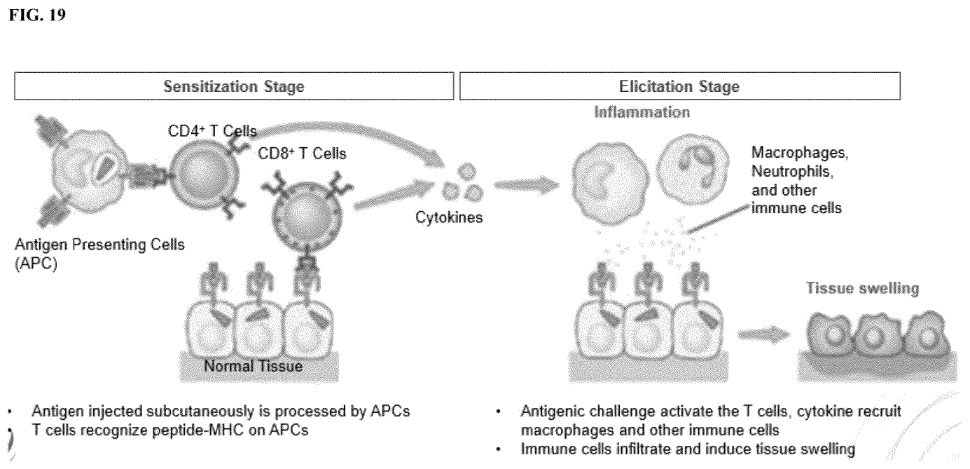

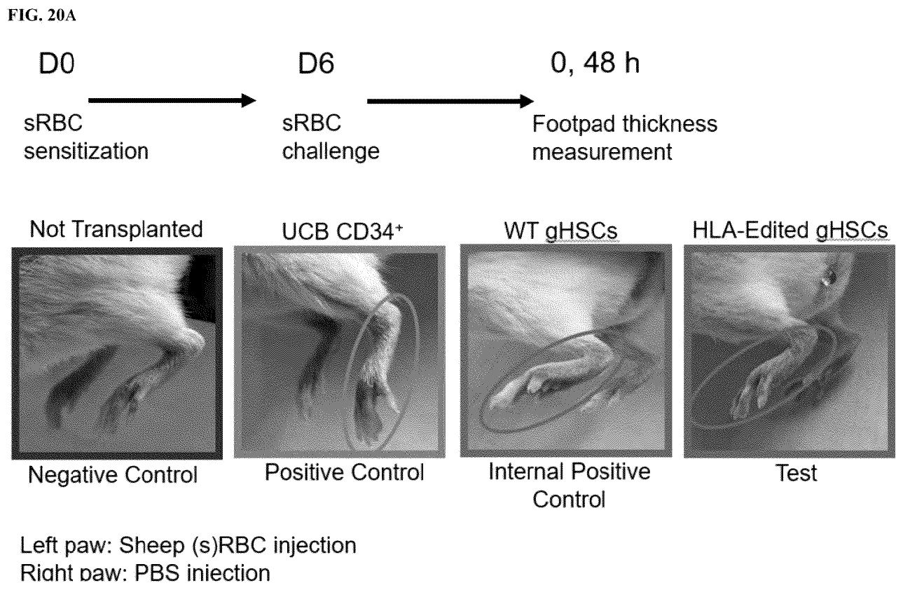

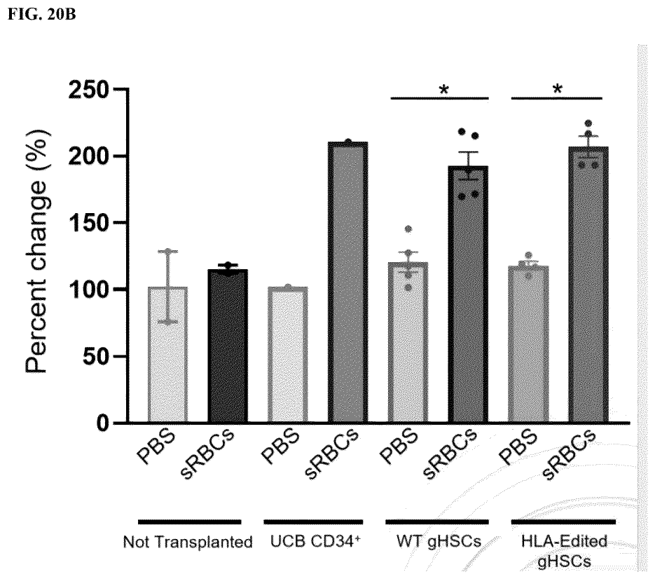

The overall expression of the other class-II molecules in HLA-DQB1 and HLA-DPB1 supported by the edited HSCs was evaluated, by evaluating the expression in macrophages differentiated from the HSCs. The design of the study is schematically shown in FIG. 16A. It was found that the deletion of HLA-DQB1 and HLA-DPB1 did not affect the expression of other HLA Class II molecules (FIG. 16B). For example, HLA-DR is comparably expressed in both WT and HLA-edited cells (FIG. 16C). In FIGS. 19B and C, CIITA-KO is as a positive control.

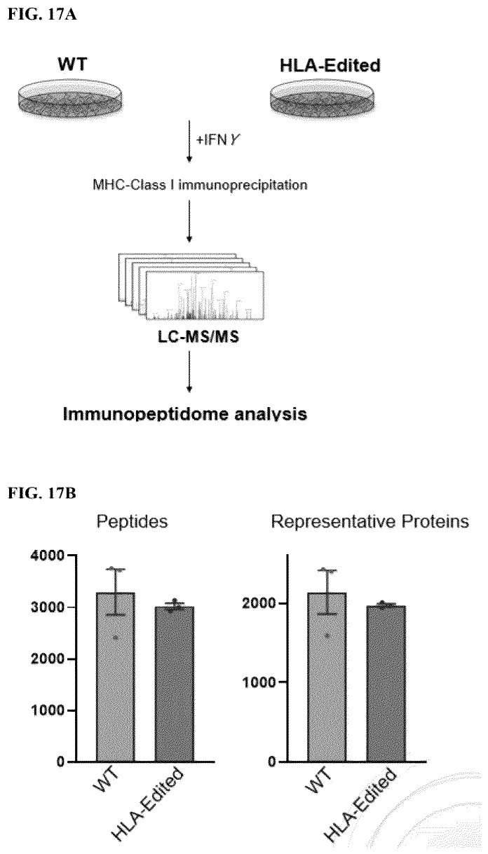

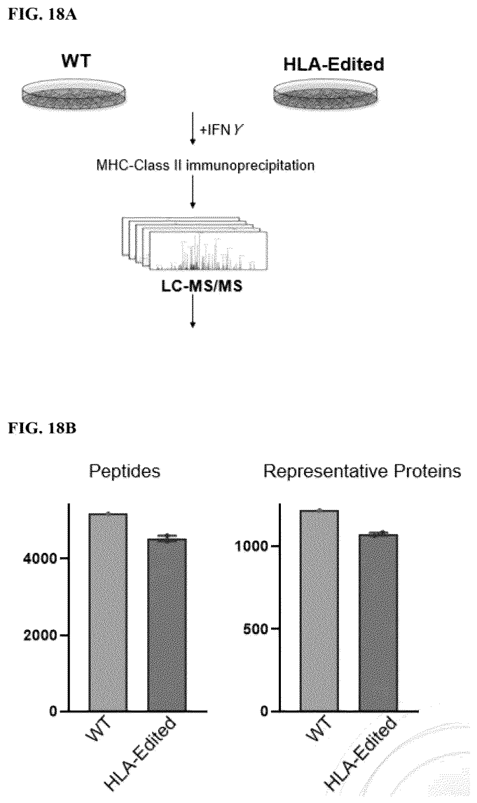

Antigen presenting cells (APC) present antigens to helper CD4⁺ T cells through the HLA-II molecules. Activation of helper CD4⁺ T cells promotes the generation of antigen-specific CD8⁺ T cells which further develop into antigen-specific CTLs. Likewise, HLA Class I molecules are expressed on the surface of all nucleated cells and display peptide fragments of proteins from within the cell to CD8+ CTLs. CTLs induce cytotoxic killing of target (infected) cells upon recognition of HLA-I-peptide complex expressed on the cell surface. Hence, a study was carried out to determine if deletion of HLA-A impacts the edited HSCs' class I peptide presentation. As shown in FIGS. 17A and B, immunopeptidome analysis shows that the deletion of HLA-A does not impact overall class I peptide presentation. HLA-A edited cells showed comparable peptide and protein presentation when compared to wild type (gHSCs) HSCs. Further, as shown in FIGS. 18A and B, deletion of HLA-DQB1 and HLA-DPB1 does not impact overall class II peptide presentation by macrophages differentiated from the HSCs. Together, these data suggest that despite the deletion of HLA-A, HLA-DQ, and HLA-DP molecules, the cells preserve their ability to present a broad spectrum of class I and II peptides.

### Example 8: In Vivo Testing of Antigen-Mediated Immune Response

FIG. 19 is a schematic illustration of a Delayed Type Hypersensitivity Reaction, showing the sensitizing and eliciting stages of an antigen presentation. Briefly, upon antigen injection, antigen is processed by antigen presenting cells (APC) and presented by MHC Class II molecules on the APC surface. CD4+ T cells recognize peptide-MHC on antigen presenting cells (APCs). Upon antigenic challenge CD4+ helper T cells are activated and cytokines recruit macrophages and other immune cells, which induce tissue swelling at the site of antigen exposure.

A delayed-type hypersensitivity assay was performed on transplanted mice. Specifically, the mice were sensitized by subcutaneous injection of sheep Red blood cells as antigen. If the mice have a functional immune system, the APCs process the antigen and present peptide antigens to CD4+ T cells. Next, the mice were challenged by subcutaneous injection of the same antigen in the left paw. At this point the T cells are activated and secrete cytokines which recruit macrophages and other immune cells at the site of antigen injection creating tissue swelling. In this assay, a functional immune system could result in the swelling of the left paw as measured with a micro caliper.

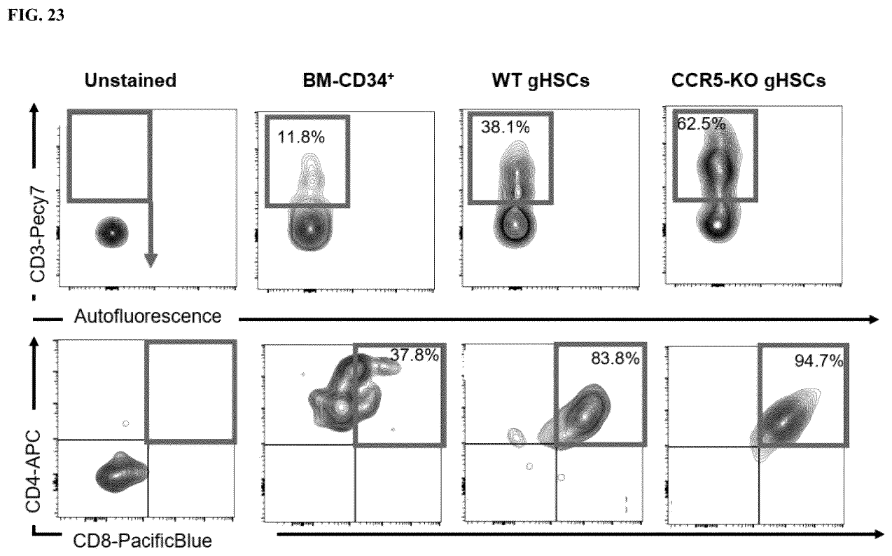

As can be seen in FIGS. 23A and B, the control (non-transplanted) mice did not show any left paw swelling as they are immunodeficient. Conversely, the mice transplanted with Cord Blood CD34+ cells showed tissue swelling and doubled the diameter of their left paw. A similar immune system response was found in both the mice transplanted with the WT (non-edited HSCs) and the HLA-edited HSCs (Triple KO).

### Example 9: In Vivo Testing of HSC-Derived T Cells

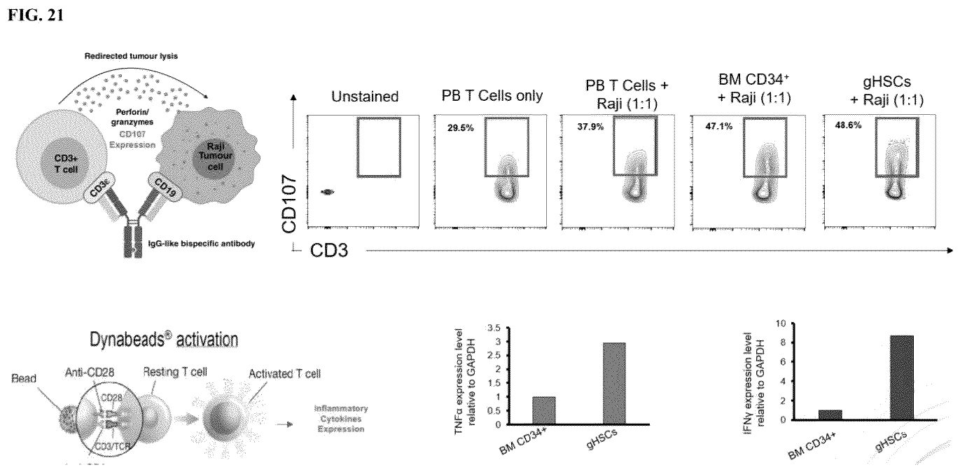

In-vitro activation of the HSC-derived T cells was measured, and results are illustrated in FIG. 21. Top panel of FIG. 21 shows FACS analysis of activated T cells from different sources, including the HSCs prepared using Piezo1 activation. T cells prepared from the HSCs demonstrated comparable or superior activation as measured by increased CD107 expression. The lower panel shows Dynabeads activation, where activated T cells express inflammatory cytokines. HSC-derived T cells prepared using Piezo1 activation expressed higher levels of inflammatory cytokines as exemplified by TNF-alpha and interferon gamma expression levels.

### Example 10: Evaluating Properties of CCR5 Knock Out HSCs to Develop into Pro-T Cells

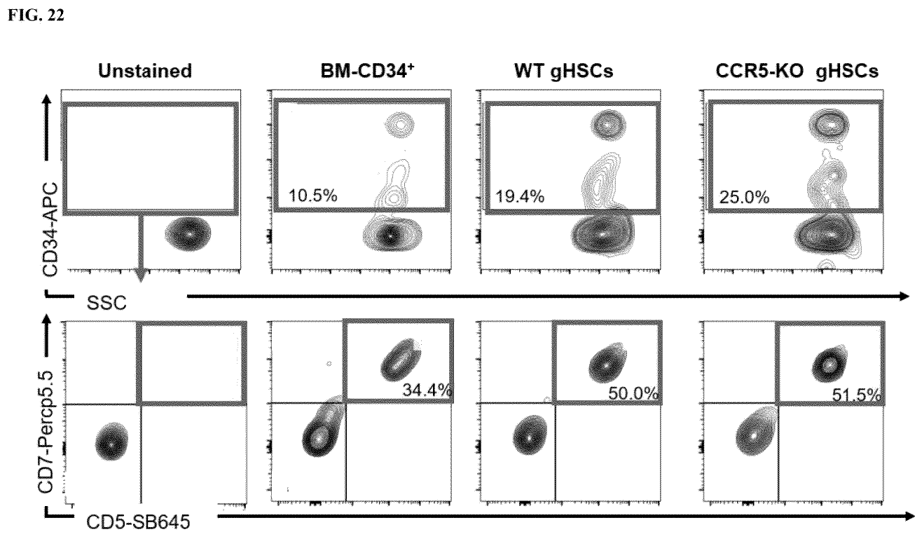

To determine if CCR5 knock out (CCR5-KO) HSCs can comparably differentiate to pro-T cells similar to their wild-type counterparts from which they are derived, a study was performed in which the CD34, CD7 and CD5 expression of the HSCs and the CCR5-KO were measured. As can be seen in FIG. 22, HSCs successfully differentiated into CD34+CD7+CD5+ pro-T cells comparably to bone marrow derived CD34+ cells. Likewise, the CCR5-KO, like their gHSC counterpart, successfully differentiated into CD34+CD7+CD5+ pro-T cells.

Next, the property of CCR5-knocked out HSCs to differentiate into double positive (CD4+CD8+) T cells was assessed. As can be seen in FIG. 23, CCR5-knocked out HSCs comparably differentiated into double positive (CD4+CD8+) T cells when compared to their gHSC counterparts from which they were derived (i.e., HSCs of the present disclosure).

### Example 11: Evaluating HSC-Derived T Cell (Pro-T Cell) Differentiation and Maturation

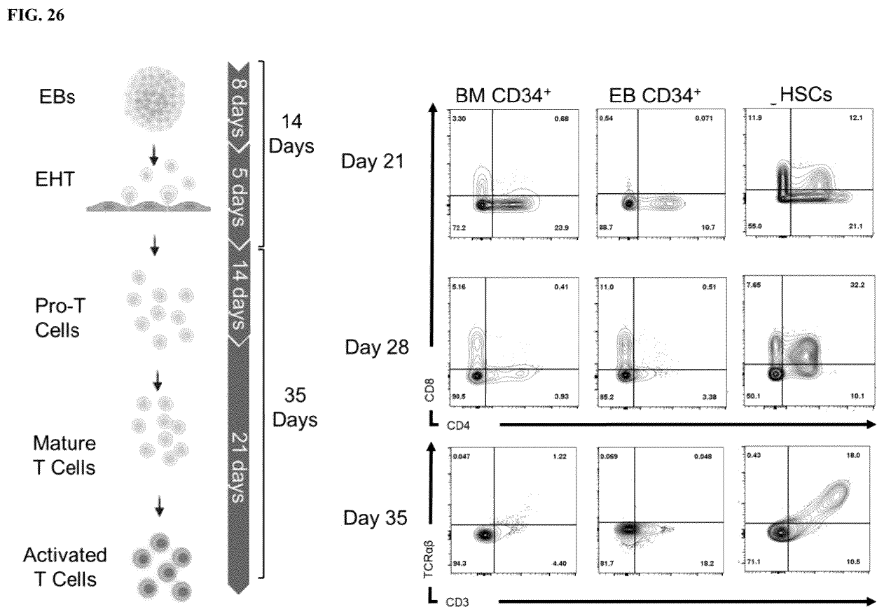

Next, the ability of the HSC-derived T cells (pro-T cells) to differentiate into mature T cells was tested. After a 35-day differentiation period, pro-T cells were evaluated by cell sorting for the presence of CD4+, CD8+, and AB+ T cell populations. As shown in FIG. 26, pro-T cells differentiated into CD4+, CD8+, and αβ+ T cells more efficiently than bone marrow (BM)-derived CD34+ cells and the CD34+ cells derived from the embryonic bodies (EB).

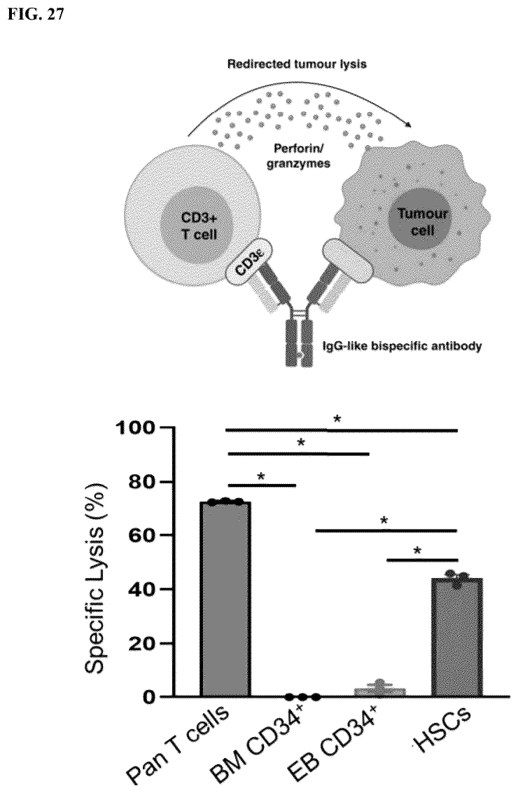

Next, to test the functional properties, each of the T cell populations were co-cultured with a CD19+ lymphoma cell line and an anti-CD3/CD-19 bispecific antibody. In this experimental model, the bispecific antibody engaged both the CD3 receptor on T cells and the CD19 cell surface receptor of the lymphoma cells, thus triggering T cell activation. The degree of activation was evaluated by measuring the subsequent T-cell mediated cytotoxicity in comparison to a Pan T cell control. As shown in FIG. 27, the pro-T cells exhibited a statistically significant outperformance in cytotoxicity in comparison to both the BM CD34+ T cells and the EB CD34+ T cells.

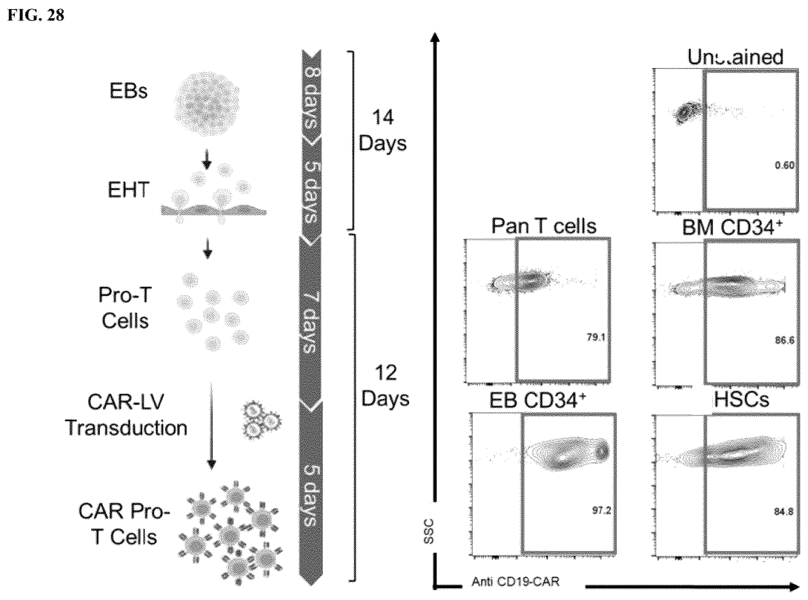

Because the overall differentiation process of pro-T cells is 35 days long, a transduction experiment was performed to test if the time required to differentiate the HSCs could be decreased. Pro-T cells were cultured in an activation media (for approx. 7 days) to increase the transduction efficiency of the cells. Next, the cells were transduced with lentiviral (LV) particles encoding an anti-CD19 CAR transgene. The cells were cultured for additional 4-5 days (a total of 12 days) and their maturation and killing capabilities were evaluated. As shown in FIG. 28, the HSC-derived pro-T cells can be transduced with high efficiency, with more than 80% of the cells express the anti-CD19 CAR as evidenced by cell sorting.

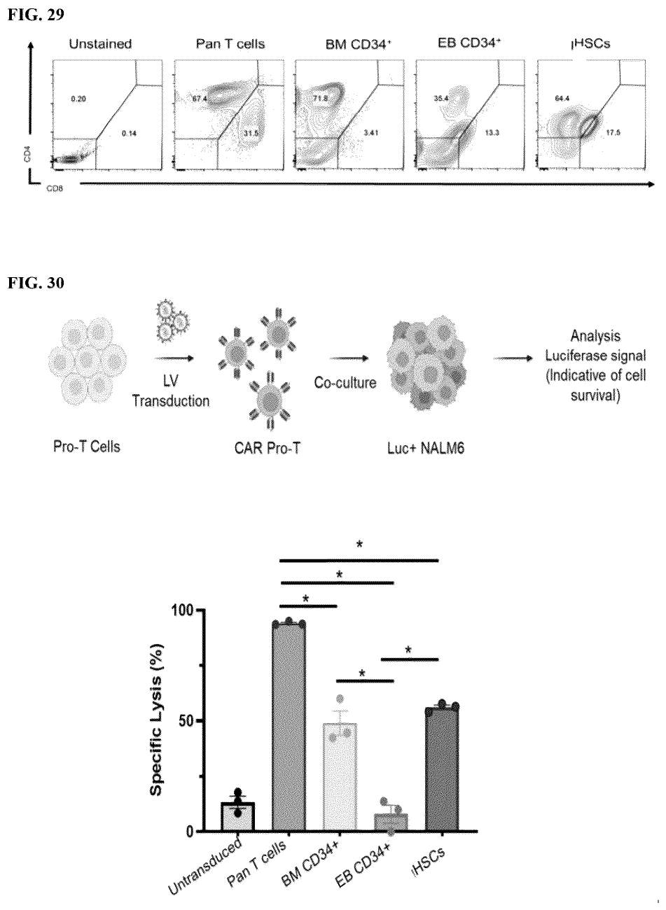

Next, the pro-T cells were evaluated for their ability to effectively mature into CD4+/CD8+ T cells via CAR transduction. The pro-T cells, along with bone marrow (BM)-derived CD34+ cells and CD34+ cells derived from the embryonic bodies (EB) (and Pan T cells as a positive control), underwent LV-transduction with the anti-CD19 CAR. The T cell subsets were screened by cell sorting for the presence of CD4 or CD8 cell surface marker expression. As shown in FIG. 29, the results indicated that CAR transduction promoted T cell maturation and that an increased degree of T cell maturation was observed in the pro-T cells in comparison to bone marrow (BM)-derived CD34+ cells and the CD34+ cells derived from the embryonic bodies (EB).

The ability of LV-transduced pro-T cells to function via anti-CD19 receptor-mediated cytotoxicity was evaluated. The T cell subsets were cocultured with a CD19+ leukemia cell line (NALM6) expressing a luciferase reporter gene (Luc+) to measure the degree of T cell-mediated cell lysis, with untransduced cells and Pan T cells as a negative and positive control, respectively. As shown in FIG. 30, the CAR pro-T cells effectively functioned via T cell-mediated lysis, demonstrating a degree of cytotoxicity comparable to the CAR-pro T cells derived from the BM CD34+ cells. Conversely, the CAR pro-T cells derived from the EB CD34+ cells showed no ability to kill the target cells.

### Example 12: Evaluating HSC Properties of Developing into Pro-T Cells

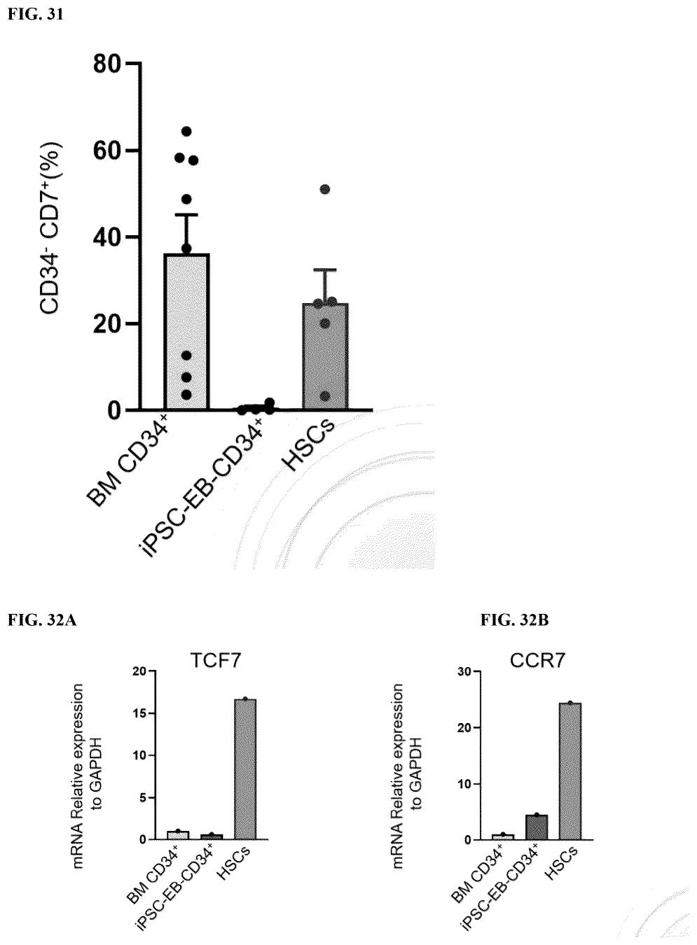

The ability of the HSCs to develop into pro-T cells was assessed by measuring the CD34−CD7+ markers on the pro-T cells. As shown in FIG. 31, FACS analysis showed that HSCs produced according to this disclosure successfully differentiated into CD34−CD7+ pro-T cells, as compared to bone marrow derived CD34+ cells or EB-derived CD34+ cells.

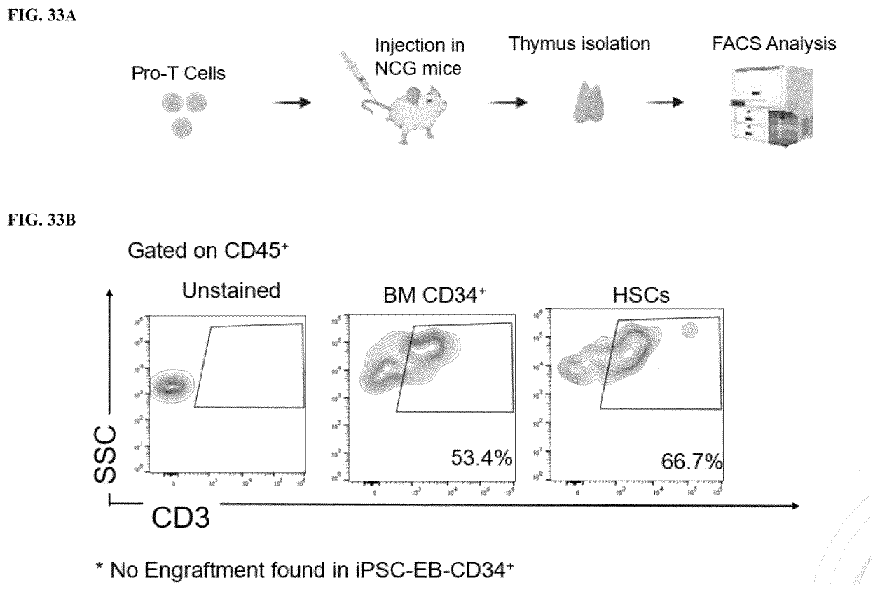

Next, the expression of T cell-specific transcription factors and thymus engrafting molecules were measured. FIG. 32A shows increased TCF7 expression and FIG. 32B shows increased CCR7 expression in the HSC-derived pro-T cells of the disclosure. FIG. 33A shows HSCs-derived Pro-T Cells engraft and differentiate in thymus. FIG. 33B shows FACS analysis of CD3 cell population of cells gated on a CD45+ cell population, which shows the superior engraftment and differentiation potential of the HSCs-derived Pro-T Cells in the thymus. Pro-T Cells of this example were prepared from HSCs using Piezo1 activation as already described.

### Example 13: HLA Editing

Further candidate gRNAs were evaluated for potential off-target editing, with gRNAs shown in Table 5. The resulting cells were tested as described in Examples 5-12, above, with substantially similar results.

TABLE 5
gRNA target sequences for disrupting HLA-A,
HLA-DPB1, HLA-DQB1, and HLA-DQB1.

| Target gene | Target sequence | PAM |
|---|---|---|
| HLA-A | ATTTCTTCACATCCGTGTCC | CGG |
| | (SEQ ID NO: 2) | |
| HLA-A | GAGGGTTCGGGGCGCCATGA | CGG |
| | (SEQ ID NO: 6) | |
| HLA-DPB1 | GGAGAGATACATCTACAACC | GGG |
| | (SEQ ID NO: 21) | |
| HLA-DQB1 | GTGCTACTTCACCAACGGGA | CGG |
| | (SEQ ID NO: 26) | |
| HLA-DQB1 | AGGTCGTGCGGAGCTCCAAC | TGG |
| | (SEQ ID NO: 27) | |

### Example 14: HLA Editing

Further candidate gRNAs were evaluated for potential off-target editing, with gRNAs shown in Table 6A-6D. The resulting cells were tested as described in Examples 5-12, above, with substantially similar results.

TABLE 6A
gRNA target sequences for disrupting HLA-A,
HLA-DPB1, HLA-DQB1, and HLA-DQB1.

| Target gene | Target sequence | PAM |
|---|---|---|
| HLA-A | ATTTCTTCACATCCGTGTCC | CGG |
| | (SEQ ID NO: 2) | |
| HLA-DPB1 | GGAGAGATACATCTACAACC | GGG |
| | (SEQ ID NO: 21) | |
| HLA-DQB1 | GTGCTACTTCACCAACGGGA | CGG |
| | (SEQ ID NO: 26) | |

TABLE 6B
gRNA target sequences for disrupting HLA-A,
HLA-DPB1, HLA-DQB1, and HLA-DQB1.

| Target gene | Target sequence | PAM |
|---|---|---|
| HLA-A | GAGGGTTCGGGGCGCCATGA | CGG |
| | (SEQ ID NO: 6) | |
| HLA-DPB1 | GGAGAGATACATCTACAACC | GGG |
| | (SEQ ID NO: 21) | |
| HLA-DQB1 | GTGCTACTTCACCAACGGGA | CGG |
| | (SEQ ID NO: 26) | |

TABLE 6C
gRNA target sequences for disrupting HLA-A, HLA-
DPB1, HLA-DQB1, and HLA-DQB1.

| Target gene | Target sequence | PAM |
|---|---|---|
| HLA-A | ATTTCTTCACATCCGTGTCC | CGG |
| | (SEQ ID NO: 2) | |
| HLA-DPB1 | GGAGAGATACATCTACAACC | GGG |
| | (SEQ ID NO: 21) | |
| HLA-DQB1 | AGGTCGTGCGGAGCTCCAAC | TGG |
| | (SEQ ID NO: 27) | |

TABLE 6D
gRNA target sequences for disrupting HLA-A, HLA-
DPB1, HLA-DQB1, and HLA-DQB1.

| Target gene | Target sequence | PAM |
|---|---|---|
| HLA-A | GAGGGTTCGGGGCGCCATGA | CGG |
| | (SEQ ID NO: 6) | |
| HLA-DPB1 | GGAGAGATACATCTACAACC | GGG |
| | (SEQ ID NO: 21) | |
| HLA-DQB1 | AGGTCGTGCGGAGCTCCAAC | TGG |
| | (SEQ ID NO: 27) | |

### ENUMERATED EMBODIMENTS

The disclosure further provides the following enumerated embodiments:
1. An HLA-modified cell that is HLA-A^{neg}, HLA-DPB1^{neg}, and HLA-DQB1^{neg}, wherein the cell is homozygous for, or comprises a single copy of, HLA-B*08:01, HLA-C*07:01, and/or HLA-DRB1*03:01.
2. The HLA-modified cell of clause 1, wherein the cell is homozygous for HLA-A*01:01, with a disruption in exon 2 of each HLA-A gene within the sequence targeted by a gRNA comprising the nucleotide sequence:

(SEQ ID NO: 6)
GAGGGTTCGGGGCGCCATGA.

3. The HLA-modified cell of clause 2, wherein the disruption in exon 2 of each HLA-A gene comprises a deletion, insertion, or indel.
4. The HLA-modified cell of clause 3, wherein the disruption is a deletion in exon 2.
5. The HLA-modified cell of any one of clauses 1 to 4, wherein the cell is homozygous or heterozygous for HLA-DPB1*01:01 or HLA-DPB1*04:01, with a disruption in exon 2 of each HLA-DPB1 gene within the nucleotide sequence targeted by a gRNA comprising the nucleotide sequence:

(SEQ ID NO: 21)
GGAGAGATACATCTACAACC.

6. The HLA-modified cell of clause 5, where the disruption in exon 2 of each HLA-DPB1 gene comprises a deletion, insertion, or indel.
7. The HLA-modified cell of clause 6, where the disruption is a deletion.
8. The HLA-modified cell of clause 6, where the disruption is an insertion.
9. The HLA-modified cell of clause 6, where the disruption is an indel.
10 The HLA-modified cell of any one of clauses 1 to 9, wherein the cell is homozygous for HLA-DQB1*02:01, with a disruption in exon 2 of each HLA-DQB1 gene within the nucleotide sequence targeted by a gRNA comprising the nucleotide sequence:

(SEQ ID NO: 26)
GTGCTACTTCACCAACGGGA
or
(SEQ ID NO: 27)
AGGTCGTGCGGAGCTCCAAC.

11. The HLA-modified cell of clause 10, where the disruption in exon 2 of each HLA-DQB1 comprises a deletion, insertion, or indel in exon 2.
12. The HLA-modified cell of clause 11, where the disruption is a deletion.
13. The HLA-modified cell of clause 11, where the disruption is an insertion.
14. The HLA-modified cell of any one of clauses 1 to 13, wherein the cell is homozygous or heterozygous for HLA-DPA1, optionally wherein the alleles comprise one or more of HLA-DPA1*01:03 and HLA-DPA1*02:01.
15. The HLA-modified cell of any one of clauses 1 to 14, wherein the cell is homozygous or heterozygous for HLA-DQA1, or comprises a single copy of HLA-DQA1.
16. The HLA-modified cell of clause 15, wherein cell comprises at least one HLA-DQA1*05:01 allele.
17. The HLA-modified cell of any one of clauses 1 to 16, wherein the cell is homozygous or heterozygous for HLA-DRB3, or comprises a single copy of HLA-DRB3.
18. The HLA-modified cell of clause 17, wherein the cell comprises at least one HLA-DRB3*01:01 allele.
19. The HLA-modified cell of any one of clauses 1 to 18, wherein the cell is homozygous for HLA-C*07:01, HLA-B*08:01, and HLA-DRB1*03:01, and optionally HLA-DRB3*01:01.
20 The HLA-modified cell of any one of clauses 1 to 19, wherein the cell is a human stem cell.
21. The HLA-modified cell of clause 20, wherein the stem cell is an induced pluripotent stem cell (iPSC), and the iPSC is derived from human CD34+ cells.
22. The HLA-modified cell of clause 20, wherein the cell is a hematopoietic stem cell (HSC), or a cell population derived therefrom.
23. The HLA-modified cell of clause 22, wherein the cell is a hematopoietic cell lineage, optionally the hematopoietic lineage is selected from common lymphoid precursor (CLP) cells, granulocyte-monocyte progenitor (GMP) cells, progenitor-T cells, T lymphocytes, B lymphocytes, Natural Killer cells, neutrophils, monocyte, macrophages, red cells, megakaryocytes, and platelets.
24. The HLA-modified cell of clause 23, wherein the cell is human stem cell or human progenitor cell, optionally selected from CMPs, CLPs, GMPs, B-cells, macrophages, T-cells, and subtypes thereof.
25. The HLA-modified cell of clause 20, wherein the cell is a non-hematopoietic stem cell, optionally a mesenchymal stem cell, neural stem cell, or epithelial stem cell, or the cell is a non-hematopoietic cell optionally selected from neurons, astrocytes, oligodendrocytes, cardiomyocytes, skeletal muscle cells, hepatocytes, pancreatic β cells, and lung epithelial cells, or progenitors thereof.
26. A pharmaceutical composition comprising a population of the HLA-modified cell according to any one of clauses 1 to 25 and a pharmaceutically acceptable carrier suitable for parenteral administration or engraftment in a recipient.
27. The pharmaceutical composition of clause 26, wherein the population of the HLA-modified cells are contained within containers suitable for cryopreservation, thawing, and/or maintaining viability of the cell lines.
28. The pharmaceutical composition of clause 26 or 27, wherein the pharmaceutically acceptable carrier comprises a cryoprotectant.
29. A method for cell therapy comprising administering to a recipient in need thereof the HLA-modified cell of any one of clauses 1 to 25, or the pharmaceutical composition of any one of clauses 26-28.
30 The method of clause 29, wherein the administered cell population, or tissue thereof, is matched with the recipient for immune compatibility at one or more of HLA-B, HLA-C, HLA-DRB1, HLA-DRB3, HLA-DRB4, and HLA-DRB5.
31 The method of clause 30, comprising matching loci comprising both alleles for HLA-DRB1.
32 The method of clause 30 or 31, comprising matching loci comprising both alleles for HLA-B and HLA-C.
33. The method of any one of clauses 30 to 32, comprising matching both alleles for HLA-DRB3.
34 The method of clause 29 to 33, wherein the HLA-modified cell population is an HSC population.
35. The method of any one of clauses 29 to 33, wherein the HLA-modified cell population is an immune cell lineage.
36 The method of clause 35, wherein the immune cell lineage is a T cell, progenitor T cell, NK cell, B-cell, monocyte, macrophage, neutrophil, monocyte, red cell, megakaryocytes, or platelet.
37. The method of clause 36, wherein the T cell lineage is a T-regulatory cell or cytotoxic T cell.
38 The method of clause 29 to 37, wherein the HLA-modified cell expresses a chimeric antigen receptor (CAR).
39 The method of any one of clauses 29 to 38, wherein the recipient has a condition selected from a hematological malignancy, aplastic anemia, hemoglobinopathy, inborn error of metabolism, and severe immunodeficiency.
40. The method of clause 39, wherein the recipient has a condition selected from acute myeloid leukemia; acute lymphoblastic leukemia; chronic myeloid leukemia; chronic lymphocytic leukemia; myeloproliferative disorder; myelodysplastic syndrome; multiple myeloma; Non-Hodgkin lymphoma; Hodgkin disease; aplastic anemia; pure red-cell aplasia; paroxysmal nocturnal hemoglobinuria; Fanconi anemia; thalassemia major; sickle cell anemia; severe combined immunodeficiency (SCID); acquired immune deficiency syndrome (AIDS); Wiskott-Aldrich syndrome; hemophagocytic lymphohistiocytosis; inborn errors of metabolism; epidermolysis bullosa; severe congenital neutropenia; Shwachman-Diamond syndrome; Diamond-Blackfan anemia; and leukocyte adhesion deficiency.
41 The method of any one of clauses 29 to 40, wherein the recipient has a condition selected from one or more of lymphopenia, cancer, immune deficiency, autoimmune disease, skeletal dysplasia, a bone marrow failure syndrome, and genetic disorder impacting the immune system.
42. The method of any one of clauses 29 to 41, wherein the recipient has undergone lympho-deleting therapy, cyto-reductive therapy, or immunomodulatory therapy prior to administration of the cell therapy.
43 The method of any one of clauses 29 to 42, wherein the cell population is a non-hematopoietic cell population.
44. The method of clause 43, wherein the cell population or tissue is selected from mesenchymal stem cells, neural stem cells, and epithelial stem cells.
45. The method of clause 44, wherein the cell population or tissue is selected from neurons, astrocytes, oligodendrocytes, cardiomyocytes, skeletal muscle cells, hepatocytes, pancreatic β cells, and lung epithelial cells, or progenitors thereof.
46. A method for making a cell population of the HLA-modified cell of to any one of clauses 1 to 25, comprising:
   providing an iPSC that comprises one or more copies of HLA-B*08:01, HLA-C*07:01, and HLA-DRB1*03:01;
   contacting the iPSC with one or more guide RNAs (gRNAs) and a CRISPR-Cas endonuclease configured to modify the iPSC at one or more of HLA-A, HLA-B, and HLA-DRB1 to prepare an HLA-modified iPSC population that is HLA-A^{neg}, HLA-DPB1^{neg}, and HLA-DQB1^{neg}.
47. The method of clause 46, wherein the iPSC is HLA-modified using a CRISPR-Cas9 endonuclease and one or more guide gRNAs as ribonucleoprotein.
48. The method of clause of any one of clauses 46 or 47, wherein the one or more gRNAs comprise a nucleotide sequence selected from

(SEQ ID NO: 6)
GAGGGTTCGGGGCGCCATGA;
(SEQ ID NO: 21)
GGAGAGATACATCTACAACC;
(SEQ ID NO: 26)
GTGCTACTTCACCAACGGGA;
and
(SEQ ID NO: 27)
AGGTCGTGCGGAGCTCCAAC.

49. The method of any one of clauses 46 to 48, wherein contacting the iPSC further comprises introducing one or more gRNAs and/or nucleoproteins using electroporation, lipid reagent, sonoporation, microprecipitation, microinjection, or transfection.
50. The method of any one of clauses 46 to 49, wherein the iPSC population is a human iPSC population derived from lymphocytes, cord blood cells, peripheral blood mononuclear cells, CD34+ cells, or human primary tissues.
51. The method of clause 50, further comprising preparing embryoid bodies (EBs) from the iPSC population.
52. The method of clause 51, further comprising dissociating the EBs and enriching for CD34+ cells to prepare a CD34+-enriched cell population.
53. The method of clause 52, further comprising inducing endothelial-to-hematopoietic transition (EHT) of the CD34+-enriched cell population to prepare a population comprising hematopoietic stem cells (HSCs) and/or hematopoietic stem progenitor cells (HSPCs), optionally harvesting CD34+ cells from the population comprising HSCs and/or HSPCs to enrich for a population undergoing EHT.
54. The method of clause 52 or 53, further comprising differentiating the CD34+-enriched cell population.
55. The method of clause 54, wherein CD34+ enrichment and endothelial-to-hematopoietic transition (EHT) is induced at Day 7 to Day 15 of iPSC differentiation, optionally wherein EHT is induced for at least 2 days and no more than 12 days.
56. The method of any one of clauses 53 to 55, wherein CD34+ cells are harvested from culture undergoing EHT, including harvesting of CD34+ floater and/or adherent cells.
57. The method of clause 53 to 56, where the induction of EHT comprises increasing the expression or activity of dnmt3b.
58. The method of clause 57, wherein increasing the expression or activity of dnmt3b comprises Piezo1 activation.
59. The method of any one of clauses 53 to 58, wherein the CD34+-enriched cells undergoing EHT are differentiated to one or more of common lymphoid precursor (CLP) cells, granulocyte-monocyte progenitor (GMP) cells, progenitor-T cells, T lymphocytes, B lymphocytes, Natural Killer cells, neutrophils, monocyte, macrophages, red cells, megakaryocytes, and platelets.
60. The method of clause 59, wherein the CD34+-enriched cells undergoing EHT are differentiated ex vivo to progenitor T cells, T cells, or NK cells.
61 The method of any one of clauses 53 to 58, wherein the CD34+-enriched cells undergoing EHT are cultured with a partial or full Notch ligand to produce a population comprising CD7+ progenitor T cells or a derivative cell population.
62. A cell population produced by the method of any one of clauses 46 to 61.

1. An HLA-modified cell that is HLA-A^{neg}, HLA-DPB1^{neg}, and HLA-DQB1^{neg}, wherein the cell is homozygous for, or comprises a single copy of, HLA-B*08:01, HLA-C*07:01, and/or HLA-DRB1*03:01.

2. The HLA-modified cell of claim 1, wherein the cell is homozygous for HLA-A*01:01, with a disruption in exon 1 of each HLA-A gene within the sequence targeted by a gRNA comprising the nucleotide sequence:
(SEQ ID NO: 6)
GAGGGTTCGGGGCGCCATGA.

3-4. (canceled)

5. The HLA-modified cell of claim 1, wherein the cell is homozygous for HLA-A*01:01, with a disruption in exon 2 of each HLA-A gene within the sequence targeted by a gRNA comprising the nucleotide sequence:
(SEQ ID NO: 2)
ATTTCTTCACATCCGTGTCC.

6-7. (canceled)

8. The HLA-modified cell of claim 1, wherein the cell is homozygous or heterozygous for HLA-DPB1*01:01 or HLA-DPB1*04:01, with a disruption in exon 2 of each HLA-DPB1 gene within the nucleotide sequence targeted by a gRNA comprising the nucleotide sequence:
(SEQ ID NO: 21)
GGAGAGATACATCTACAACC.

9-12. (canceled)

13. The HLA-modified cell of claim 1, wherein the cell is homozygous for HLA-DQB1*02:01, with a disruption in exon 2 of each HLA-DQB1 gene within the nucleotide sequence targeted by a gRNA comprising the nucleotide sequence:
(SEQ ID NO: 26)
GTGCTACTTCACCAACGGGA.

14. The HLA-modified cell of claim 1, wherein the cell is homozygous for HLA-DQB1*02:01, with a disruption in exon 2 of each HLA-DQB1 gene within the nucleotide sequence targeted by a gRNA comprising the nucleotide sequence: or
(SEQ ID NO: 27)
AGGTCGTGCGGAGCTCCAAC.

15-17. (canceled)

18. The HLA-modified cell of claim 1, wherein the cell is homozygous for HLA-A*01:01, with a disruption in exon 2 of each HLA-A gene within the sequence targeted by a gRNA comprising the nucleotide sequence: ATTTCTTCACATCCGTGTCC (SEQ ID NO: 2); the cell is homozygous or heterozygous for HLA-DPB1*01:01 or HLA-DPB1*04:01, with a disruption in exon 2 of each HLA-DPB1 gene within the nucleotide sequence targeted by a gRNA comprising the nucleotide sequence: GGAGAGATACATCTACAACC (SEQ ID NO: 21); and the cell is homozygous for HLA-DQB1*02:01, with a disruption in exon 2 of each HLA-DQB1 gene within the nucleotide sequence targeted by a gRNA comprising the nucleotide sequence:
(SEQ ID NO: 26)
GTGCTACTTCACCAACGGGA.

19. The HLA-modified cell of claim 1, wherein the cell is homozygous for HLA-A*01:01, with a disruption in exon 1 of each HLA-A gene within the sequence targeted by a gRNA comprising the nucleotide sequence: GAGGGTTCGGGGCGCCATGA (SEQ ID NO: 6); the cell is homozygous or heterozygous for HLA-DPB1*01:01 or HLA-DPB1*04:01, with a disruption in exon 2 of each HLA-DPB1 gene within the nucleotide sequence targeted by a gRNA comprising the nucleotide sequence: GGAGAGATACATCTACAACC (SEQ ID NO: 21); and the cell is homozygous for HLA-DQB1*02:01, with a disruption in exon 2 of each HLA-DQB1 gene within the nucleotide sequence targeted by a gRNA comprising the nucleotide sequence:
(SEQ ID NO: 26)
GTGCTACTTCACCAACGGGA.

20. The HLA-modified cell of claim 1, wherein the cell is homozygous for HLA-A*01:01, with a disruption in exon 2 of each HLA-A gene within the sequence targeted by a gRNA comprising the nucleotide sequence: ATTTCTTCACATCCGTGTCC (SEQ ID NO: 2); the cell is homozygous or heterozygous for HLA-DPB1*01:01 or HLA-DPB1*04:01, with a disruption in exon 2 of each HLA-DPB1 gene within the nucleotide sequence targeted by a gRNA comprising the nucleotide sequence: GGAGAGATACATCTACAACC (SEQ ID NO: 21); and the cell is homozygous for HLA-DQB1*02:01, with a disruption in exon 2 of each HLA-DQB1 gene within the nucleotide sequence targeted by a gRNA comprising the nucleotide sequence: or AGGTCGTGCGGAGCTCCAAC (SEQ ID NO: 27).

21. The HLA-modified cell of claim 1, wherein the cell is homozygous for HLA-A*01:01, with a disruption in exon 1 of each HLA-A gene within the sequence targeted by a gRNA comprising the nucleotide sequence: GAGGGTTCGGGGCGCCATGA (SEQ ID NO: 6); the cell is homozygous or heterozygous for HLA-DPB1*01:01 or HLA-DPB1*04:01, with a disruption in exon 2 of each HLA-DPB1 gene within the nucleotide sequence targeted by a gRNA comprising the nucleotide sequence: GGAGAGATACATCTACAACC (SEQ ID NO: 21); and the cell is homozygous for HLA-DQB1*02:01, with a disruption in exon 2 of each HLA-DQB1 gene within the nucleotide sequence targeted by a gRNA comprising the nucleotide sequence: or AGGTCGTGCGGAGCTCCAAC (SEQ ID NO: 27).

22-27. (canceled)

28. The HLA-modified cell of claim 19, wherein the cell is a human stem cell.

29. The HLA-modified cell of claim 28, wherein the stem cell is an induced pluripotent stem cell (iPSC), and the iPSC is derived from human CD34+ cells.

30. The HLA-modified cell of claim 19, wherein the cell is a hematopoietic stem cell (HSC), or a cell population derived therefrom.

31. The HLA-modified cell of claim 19, wherein the cell is a hematopoietic cell lineage cell.

32-33. (canceled)

34. A pharmaceutical composition comprising a population of the HLA-modified cell according to claim 1 and a pharmaceutically acceptable carrier suitable for parenteral administration or engraftment in a recipient.

35. (canceled)

36. The pharmaceutical composition of claim 34, wherein the pharmaceutically acceptable carrier comprises a cryoprotectant.

37. A method for cell therapy comprising administering to a recipient in need thereof the HLA-modified cell of claim 1.

38. The method of claim 37, wherein the administered cell population, or tissue thereof, is matched with the recipient for immune compatibility at one or more of HLA-B, HLA-C, HLA-DRB1, HLA-DRB3, HLA-DRB4, and HLA-DRB5.

39-41. (canceled)

42. The method of claim 37, wherein the HLA-modified cell population is an HSC population.

43-53. (canceled)

54. A method for making a cell population of the HLA-modified cell of claim 1, comprising:
providing an induced pluripotent stem cell (iPSC) that (optionally) comprises one or more copies of HLA-B*08:01, HLA-C*07:01, and HLA-DRB1*03:01;
contacting the iPSC with one or more guide RNAs (gRNAs) and a CRISPR-Cas endonuclease configured to modify the iPSC at one or more of HLA-A, HLA-B, and HLA-DRB1 to prepare an HLA-modified iPSC population that is HLA-A^{neg}, HLA-DPB1^{neg}, and HLA-DQB1^{neg}.

55. The method of claim 54, wherein the iPSC is HLA-modified using a CRISPR-Cas9 endonuclease and one or more guide gRNAs as ribonucleoprotein.

56. The method of claim 55, wherein the one or more gRNAs comprise a nucleotide sequence selected from
(SEQ ID NO: 6)
GAGGGTTCGGGGCGCCATGA;
(SEQ ID NO: 2)
ATTTCTTCACATCCGTGTCC;
(SEQ ID NO: 21)
GGAGAGATACATCTACAACC;
(SEQ ID NO: 26)
GTGCTACTTCACCAACGGGA;
and
(SEQ ID NO: 27)
AGGTCGTGCGGAGCTCCAAC.

57. The method of claim 54, wherein the iPSC is HLA-modified using a CRISPR-Cas9 endonuclease and one or more guide gRNAs as ribonucleoprotein, wherein the one or more gRNAs comprise a nucleotide sequence selected from
(SEQ ID NO: 6)
GAGGGTTCGGGGCGCCATGA;
or
(SEQ ID NO: 2)
ATTTCTTCACATCCGTGTCC;
(SEQ ID NO: 21)
GGAGAGATACATCTACAACC;
and
(SEQ ID NO: 26)
GTGCTACTTCACCAACGGGA;
or
(SEQ ID NO: 27)
AGGTCGTGCGGAGCTCCAAC.

58-71. (canceled)


SEQUENCE LISTING (33 sequences)

SEQ ID NO: 1 - RNA, 20 nt, synthetic construct
  source 1..20: mol_type = other RNA
cgtagcccacggcgatgaag

SEQ ID NO: 2 - RNA, 20 nt, synthetic construct
  source 1..20: mol_type = other RNA
atttcttcacatccgtgtcc

SEQ ID NO: 3 - RNA, 20 nt, synthetic construct
  source 1..20: mol_type = other RNA
gtctcctggtcccaatactc

SEQ ID NO: 4 - RNA, 20 nt, synthetic construct
  source 1..20: mol_type = other RNA
gtagcccacggcgatgaagc

SEQ ID NO: 5 - RNA, 20 nt, synthetic construct
  source 1..20: mol_type = other RNA
tagcccacggcgatgaagcg

SEQ ID NO: 6 - RNA, 20 nt, synthetic construct
  source 1..20: mol_type = other RNA
gagggttcggggcgccatga

SEQ ID NO: 7 - RNA, 20 nt, synthetic construct
  source 1..20: mol_type = other RNA
ccggaacacacggaatgtga

SEQ ID NO: 8 - RNA, 20 nt, synthetic construct
  source 1..20: mol_type = other RNA
acagcgacgccgcgagccag

SEQ ID NO: 9 - RNA, 20 nt, synthetic construct
  source 1..20: mol_type = other RNA
ccactcacagattgaccgag

SEQ ID NO: 10 - RNA, 20 nt, synthetic construct
  source 1..20: mol_type = other RNA
cgacgccgcgagccagagga

SEQ ID NO: 11 - RNA, 20 nt, synthetic construct
  source 1..20: mol_type = other RNA
cagattgaccgagtggacct

SEQ ID NO: 12 - RNA, 20 nt, synthetic construct
  source 1..20: mol_type = other RNA
cagactgaccgagtggacct

SEQ ID NO: 13 - RNA, 20 nt, synthetic construct
  source 1..20: mol_type = other RNA
acagactgaccgagtggacc

SEQ ID NO: 14 - RNA, 20 nt, synthetic construct
  source 1..20: mol_type = other RNA
gccgggccgggacacggatg

SEQ ID NO: 15 - RNA, 20 nt, synthetic construct
  source 1..20: mol_type = other RNA
ccaggagacacggaatgtga

SEQ ID NO: 16 - RNA, 20 nt, synthetic construct
  source 1..20: mol_type = other RNA
ccagtcacagactgaccgag

SEQ ID NO: 17 - RNA, 20 nt, synthetic construct
  source 1..20: mol_type = other RNA
tcgacagcgacgccgcgagc

SEQ ID NO: 18 - RNA, 20 nt, synthetic construct
  source 1..20: mol_type = other RNA
gagatacatctacaaccggg

SEQ ID NO: 19 - RNA, 20 nt, synthetic construct
  source 1..20: mol_type = other RNA
tggagagatacatctacaac

SEQ ID NO: 20 - RNA, 20 nt, synthetic construct
  source 1..20: mol_type = other RNA
ggttgtagatgtatctctcc

SEQ ID NO: 21 - RNA, 20 nt, synthetic construct
  source 1..20: mol_type = other RNA
ggagagatacatctacaacc

SEQ ID NO: 22 - RNA, 20 nt, synthetic construct
  source 1..20: mol_type = other RNA
aggaatgctacgcgtttaat

SEQ ID NO: 23 - RNA, 20 nt, synthetic construct
  source 1..20: mol_type = other RNA
cagatacatctataaccgag

SEQ ID NO: 24 - RNA, 20 nt, synthetic construct
  source 1..20: mol_type = other RNA
cgagtactggaacagccaga

SEQ ID NO: 25 - RNA, 20 nt, synthetic construct
  source 1..20: mol_type = other RNA
cccgttggtgaagtagcaca

SEQ ID NO: 26 - RNA, 20 nt, synthetic construct
  source 1..20: mol_type = other RNA
gtgctacttcaccaacggga

SEQ ID NO: 27 - RNA, 20 nt, synthetic construct
  source 1..20: mol_type = other RNA
aggtcgtgcggagctccaac

SEQ ID NO: 28 - RNA, 20 nt, synthetic construct
  source 1..20: mol_type = other RNA
aggtgacagttgctacccga

SEQ ID NO: 29 - RNA, 20 nt, synthetic construct
  source 1..20: mol_type = other RNA
tcccgttggtgaagtagcac

SEQ ID NO: 30 - RNA, 20 nt, synthetic construct
  source 1..20: mol_type = other RNA
aactacgaggtggcgtaccg

SEQ ID NO: 31 - RNA, 20 nt, synthetic construct
  source 1..20: mol_type = other RNA
ctgttccagtactcggcaac

SEQ ID NO: 32 - RNA, 20 nt, synthetic construct
  source 1..20: mol_type = other RNA
actacgaggtggcgtaccgc

SEQ ID NO: 33 - RNA, 20 nt, synthetic construct
  source 1..20: mol_type = other RNA
cgaagcgcacgtactcctct